SEQUENCE LISTING (22 sequences)

SEQ ID NO: 1 - DNA, 24 bp, Artificial Sequence
  Human RT GAPDH forward primer
agcctcaagatcatcagcaatgcc

SEQ ID NO: 2 - DNA, 24 bp, Artificial Sequence
  Human RT GAPDH Reverse Primer
tgtggtcatgagtccttccacgat

SEQ ID NO: 3 - DNA, 24 bp, Artificial Sequence
  Mouse RT HuR Forward Primer
actgcagggatgacattgggagaa

SEQ ID NO: 4 - DNA, 24 bp, Artificial Sequence
  Mouse RT HuR Reverse Primer
aagctttgcagattcaacctcgcc

SEQ ID NO: 5 - DNA, 24 bp, Artificial Sequence
  Human RT HuR Forward Primer
atgaagaccacatggccgaagact

SEQ ID NO: 6 - DNA, 24 bp, Artificial Sequence
  Human RT HuR Reverse Primer
agttcacaaagccatagcccaagc

SEQ ID NO: 7 - DNA, 24 bp, Artificial Sequence
  Human RT CD9 Forward Primer
tcagaccaagagcatcttcgagca

SEQ ID NO: 8 - DNA, 24 bp, Artificial Sequence
  Human RT CD9 Reverse Primer
accaagaggaagccgaagaacagt

SEQ ID NO: 9 - DNA, 24 bp, Artificial Sequence
  Human RT CALM2 Forward Primer
ctgaccaactgactgaagagcaga

SEQ ID NO: 10 - DNA, 23 bp, Artificial Sequence
  Human RT CALM2 Reverse Primer
ttctgtgggattctgcccaagag

SEQ ID NO: 11 - DNA, 18 bp, Artificial Sequence
  shRNA H760 directed against HuR
ggatcctctggcagatgt

SEQ ID NO: 12 - DNA, 33 bp, Bacteriophage T7
ccaagcttctaatacgactcacttatagggaga

SEQ ID NO: 13 - DNA, 22 bp, Artificial Sequence
  CD9 CR 118-560 Forward Primer
tcaaaggaggcaccaagtgcat

SEQ ID NO: 14 - DNA, 22 bp, Artificial Sequence
  CD9 CR 118-560 Reverse Primer
aacgcatagtggatggctttca

SEQ ID NO: 15 - DNA, 22 bp, Artificial Sequence
  CD9 3′UTR 798-1231 Forward Primer
agtcagcttacatccctgagca

SEQ ID NO: 16 - DNA, 29 bp, Artificial Sequence
  CD9 3′UTR 798-1231 Reverse Primer
gacattgtcataattttttattatgtatc

SEQ ID NO: 17 - DNA, 20 bp, Artificial Sequence
  CALM2 CR 72-515 Forward Primer
gctgaccaactgactgaaga

SEQ ID NO: 18 - DNA, 24 bp, Artificial Sequence
  CALM2 CR 72-515 Reverse Primer
ctttgctgtcatcatttgtacaaa

SEQ ID NO: 19 - DNA, 23 bp, Artificial Sequence
  CALM2 3′UTR 518-1128 Forward Primer
agaccttgtacagaatgtgttaa

SEQ ID NO: 20 - DNA, 27 bp, Artificial Sequence
  CALM2 3′UTR 518-1128 Reverse Primer
gggtaaattgtaatttttttattggaa

SEQ ID NO: 21 - DNA, 20 bp, Artificial Sequence
  GAPDH 3′UTR Forward Primer
cctcaacgaccactttgtca

SEQ ID NO: 22 - DNA, 23 bp, Artificial Sequence
  GAPDH 3′UTR Reverse Primer
ggttgagcacagggtactttatt

(19) United States
(12) Patent Application Publication
(10) Pub. No.: US 2011/0223616 A1
(43) Pub. Date: Sep. 15, 2011

(54) HuR-Associated Biomarkers

(75) Inventors: Ulus Atasoy, Columbia, MO (US); Robert Calaluce, Columbia, MO (US); Justin Wade Davis, Columbia, MO (US)

(73) Assignee: THE CURATORS OF THE UNIVERSITY OF MISSOURI, Columbia, MO (US)

(21) Appl. No.: 13/046,201

(22) Filed: Mar. 11, 2011

Related U.S. Application Data
(60) Provisional application No. 61/313,463, filed on Mar. 12, 2010.

(51) Int. Cl. G01N 33/573; C07K 14/47; C12N 9/14; C07H 21/02; G01N 33/68; G01N 33/53

(52) U.S. Cl. 435/7.4; 530/350; 435/195; 536/23.2; 536/24.5; 436/501; 436/94

(57) ABSTRACT

The present invention relates to methods of identifying gene targets, including methods of using ribonucleoprotein (RNP) immunoprecipitation-microarrays to identify cancer gene targets, such as subsets of RNP-associated mRNAs in breast cancer cell lines. Also presented, are ribonucleotide binding protein-associated biomarkers, panels or sets of ribonucleotide binding protein-associated biomarkers, methods and compositions comprising ribonucleotide-binding protein, associated nucleotides, nucleotide arrays, and kits to facilitate the diagnosis of and monitoring the disease status or progression of treatment of breast cancers, including drug-resistant breast cancers.

### CROSS-REFERENCE TO RELATED APPLICATIONS

The pending application claims priority claims under 35 U.S.C. §119(e) to U.S. Provisional Patent Application No. 61/313,463, filed Mar. 12, 2010, the disclosure of which is incorporated herein by reference.

### STATEMENT REGARDING FEDERALLY SPONSORED RESEARCH OR DEVELOPMENT

This disclosure was made with government support under contract W81XWH-07-1-0406 awarded by the Department of Defense. The U.S. Government has certain rights in the invention.

### INCORPORATION BY REFERENCE OF MATERIAL SUBMITTED ON A COMPACT DISK

The sequence listing contained in the file “UMCO D604U1 (09UMC071)_ST25.txt” modified on Mar. 9, 2011, having a file size of 4,724 bytes, is incorporated by reference in its entirety herein.

### FIELD

Presented are methods of identifying gene targets, including methods of using ribonucleoprotein (RNP) immunoprecipitation-microarrays to identify cancer gene targets, such as subsets of RNP-associated mRNAs in breast cancer cell lines. Also presented, are ribonucleotide binding protein-associated biomarkers, panels or sets of ribonucleotide binding protein-associated biomarkers, methods and compositions comprising ribonucleotide-binding protein-associated nucleotides, nucleotide arrays, and kits to facilitate the diagnosis of and monitoring the disease status or progression of treatment of breast cancers, including drug-resistant breast cancers.

### BACKGROUND

Over the past decade, array technologies have provided several new means for profiling global changes in gene expression. The power of DNA microarrays is perhaps best illustrated in the way it has been used to differentiate treatment responses in patient populations. Individualized and targeted therapy for several tumors, based upon underlying differences at the molecular level among gene expression profiles, is beginning to replace traditional morphological-based treatment models [Dietel M et al., Arch 2006, 448(6):744-755; Mischel P S et al., Nat Rev Neurosci 2004, 5(10):782-792; N Engl J Med 2006, 355(26):2783-2785]. Genome-wide microarray analyses, however, are inherently flawed since they globally profile the steady-state levels of mRNAs, referred to as the transcriptome. Cellular protein expression levels, however, do not directly correlate with steady-state levels of mRNAs. It is well accepted that there is a poor correlation between steady-state RNA levels and protein levels. This discordance has been attributed to post-transcriptional control mechanisms affecting mRNA stability and translation. Steady-state mRNA levels of genes controlled partially or totally at this level may be misleading. Gygi and colleagues, for example, have shown that correlations between mRNA and protein levels could not be predicted from information about mRNA steady-state levels alone [Mol Cell Biol 1999, 19(3):1720-1730]. They observed that some genes had the same mRNA levels, but protein levels varied more than 20-fold. Conversely, some proteins had equal expression levels, but their respective mRNA levels varied by more than 30-fold. They concluded that “transcript levels provide little predictive value with respect to the extent of protein expression” [Gygi SP et al., Mol Cell Biol 1999, 19(3):1720-1730]. Idekar and colleagues have also described similar results for the galactose gene [Ideker T et al., Science 2001, 292(5518):929-934].

Although our understanding of transcriptional gene regulation is advanced, post-transcriptional gene regulation remains largely unexplored. It is becoming clear, however, that this is an important mode of gene regulation, particularly for proinflammatory genes. These genes appear to be regulated at a post-transcriptional level by RNA binding proteins (RBPs), which interact with AU-rich elements (AREs) in the 3′ untranslated region (UTR) of mRNAs. Approximately 3,000 human genes contain AREs, representing 8% of the human genome [Khabar K S et al., Genomics 2005, 85(2):165-175]. Many of the genes which possess AREs are involved in areas of transient biological responses including cell growth and differentiation, immune responses, signal transduction, transcriptional and translational control, hematopoiesis, apoptosis, nutrient transport, and metabolism [Khabar K S et al., Genomics 2005, 85(2):165-175; Khabar K S, J Interferon Cytokine Res 2005, 25(1):1-10].

New methods have revealed the identification of in vivo mRNA targets of different RBPs on a global scale. The ribonomic approach involves several steps, including immunoprecipitation of ribonuclear particle complexes (RNPs) with antibodies directed against different RBPs, extraction of mRNAs, and hybridization of the mRNAs to microarrays [Intine R V et al., Mol Cell 2003, 12(5):1301-1307; Tenenbaum et al., Proc Natl Acad Sci USA 2000, 97(26):14085-14090; Tenenbaum S A et al., Methods 2002, 26(2):191-198]. This “RIP-Chip” approach enables investigators to identify groups of post-transcriptionally regulated mRNAs, which are coordinately controlled by RBPs during various biological processes. A new model has been developed which states that RBPs coordinately regulate the expression of biologically related molecules [Keene J D, Nat Genet 2003, 33(2):111-112; Keene J D, Tenenbaum S A, Mol Cell 2002, 9(6):1161-1167]. The “post-transcriptional operon hypothesis” is being confirmed in many different laboratories, broadening our understanding of post-transcriptional regulation as putative operons are characterized at a molecular level [Intine R V et al., Mol Cell 2003, 12(5):1301-1307; Gerber A P et al., PLoS Biol 2004, 2(3):E79; Grigull J et al., Mol Cell Biol 2004, 24(12):5534-5547; Hieronymus H, Silver P A, Nat Genet 2003, 33(2):155-161; Hieronymus H et al., Genes Dev 2004, 18(21):2652-2662; Rajasekhar V K, Holland E C, Oncogene 2004, 23(18):3248-3264].

HuR is a RBP that binds to AREs of many proto-oncogenes and labile mRNAs. It has emerged as a key regulatory factor which stabilizes and translationally enhances its targets mRNAs, and affects their transport from the nucleus to the cytoplasm [Atasoy U et al., J Cell Sci 1998, 111 (Pt 21):3145-3156; Fan X C, Steitz J A, Embo J 1998, 17(12):3448-3460; Ma W J et al., J Biol Chem 1996, 271(14):8144-8151]. HuR belongs to the ELAV (embryonic lethal abnormal vision) family found in mammalian cells containing four members: HuR, HuB, HuC, and HuD. HuR is the only ubiquitously-expressed member. The others are found primarily in the central nervous system and gonadal tissue [Atasoy U et al., J Cell Sci 1998, 111 (Pt 21):3145-3156]. Many HuR targets are cytokines, chemokines, and other early-response genes [Meisner N C et al., Chembiochem 2004, 5(10):1432-1447; Brennan C M, Steitz J A, Cell Mol Life Sci 2001, 58(2):266-277].

HuR has been demonstrated to control expression of genes in multiple areas of malignant transformation, one of the hallmarks of cancer first described by Hanahan and Weinberg [Cell 2000, 100(1):57-70]. Subsequent studies have suggested that HuR plays a role as a tumor maintenance gene, permissive for malignant transformation, tumor growth, and perhaps metastasis [Lopez de Silanes I et al., RNA Biol 2005, 2(1):11-13]. HuR has also been described in the literature as controlling the expression of many cancer-relevant genes, including those that encode proteins such as Prothymosin-α, Bcl-2, Mcl-1, SirT1, TGF-β, MMP-9, MTC-1, uPA, VEGF-α, HIF1-α and cyclins A, B1 and D1 [Abdelmohsen K et al., Cell Cycle 2007, 6(11):1288-1292; Abdelmohsen K et al., Mol Cell 2007, 25(4):543-557; Lal A et al., Embo J 2005, 24(10):1852-1862; Lal A et al., EMBO J 2004, 23(15):3092-3102; Levy A P, Trends Cardiovasc Med 1998, 8(6):246-250; Lopez de Silanes I et al., Proc Natl Acad Sci USA 2004, 101(9):2987-2992; Nabors L B et al., Cancer Res 2001, 61(5):2154-2161; Sheflin L G et al., Biochem Biophys Res Commun 2004, 322(2):644-651; Tran H et al., Mol Cell Biol 2003, 23(20):7177-7188; Wang W et al., EMBO J 2000, 19(10):2340-2350; Wang W et al., Mol Cell Biol 2001, 21(17):5889-5898]. Increased levels of HuR have been associated with more aggressive breast cancers, which have a more serious progression and outcome [Denkert C et al., Clin Cancer Res 2004, 10(16):5580-5586; Heinonen M et al., Cancer Res 2005, 65(6):2157-2161; Heinonen M et al., Clin Cancer Res 2007, 13(23):6959-6963].

HuR has also been described as post-transcriptionally regulating the expression of many breast cancer-relevant genes, including those that encode Glut-1, ERα, COX-2, IL-8, Cyclin E1, and BRCA-1 [Denkert C et al., Clin Cancer Res 2004, 10(16):5580-5586, Gantt K R et al., J Cell Biochem 2006, 99(2):565-574; Guo X, Hartley R S, Cancer Res 2006, 66(16):7948-7956; Kang S S et al., Jpn J Cancer Res 2002, 93(10):1123-1128; Pryzbylkowski P et al., Breast Cancer Res Treat 2008, 111(1):15-25; Saunus J M et al., Cancer Res 2008, 68(22):9469-9478; Suswam E A et al., Int J Cancer 2005, 113(6):911-919]. HuR RIP-Chip analysis has recently identified Thrombospondin 1 as a key HuR target in the MCT-1 transformed estrogen receptor positive (ER+) cell line, MCF-7 [Mazan-Mamczarz K et al., Oncogene 2008, 27: 6151-6163]. Its interactions, however, are complex, and at times, HuR may interact with miRNAs, such as Let-7, to translationally suppress the expression of C-MYC mRNAs [Kim H H et al., Genes & Dev 2009, 23: 1743-1748].

In view of the emerging role that HuR appears to have in influencing the expression of many cancer-relevant genes, we were interested in determining whether HuR was involved in coordinately regulating the expression of breast cancer genes in ER+ and ER− breast cancers. We performed a HuR RIP-Chip analysis on MDA-MB-231 (ER−) and MCF-7 (ER+) cell lines to identify cancer-relevant genes not known to be regulated by HuR, and to identify potential novel breast cancer targets. Our studies indicated that HuR was associated with unique subsets of mRNAs in each cell line, as well as a subset of HuR-associated mRNA targets common to both. We chose two cancer-associated genes, CD9 and CALMODULIN 2 (CALM2), highly expressed in both cell lines, and functionally validated the role of HuR in regulating their expression. Unexpectedly, HuR differentially regulated the same target, CD9, in both cell lines in an opposite manner. Moreover, we found presumptive differential regulation of CALM2 by HuR, as HuR interacted only with CALM2 mRNA, but not with family members CALM1 and CALM3 mRNAs. We discovered that HuR interacts with many breast cancer-relevant genes, not previously known to be controlled by HuR, and target genes which have not been shown to be cancer-related. This latter category may represent novel cancer genes discovered by HuR RIP-Chip analysis.

Clinical tests based on molecular analysis of key nucleotide or protein biomarkers have been widely used to study pathogenic disease processes and evaluate responses to drug therapy procedures. Biomarkers have also been used to predict susceptibility of an individual to specific diseases and to predict responses to drug treatments. Approaches based on the detection and statistical analysis of multiple biomarkers greatly facilitate the identification of key factors involved in the development of complex disease states, and their treatment.

While many established testing schemes rely upon methods to measure changes in specific nucleotide, protein, or metabolite levels, very few can match the power of nucleotide microarrays to facilitate the evaluation of biomarker analyses in parallel, or the ability of mass spectrometry to identify large numbers of proteins or other components in complex sample mixtures. Methods of using microarray analysis to monitor the level of mRNAs within a cell, however, often miss genes which are regulated primarily at the level of mRNA stability and translation, due to the poor correlation between steady-state mRNA levels and protein products. Therefore, there is a need to provide a new and improved method to identify, en masse, novel RBP targets associated with cancer genes in vivo from representative cell lines and clinical samples, using methods which facilitate the evaluation of a wide variety of biomarkers.

### SUMMARY

Provided are methods to identify novel biomarkers, which can be used in screening or diagnostic tests. The method may also identify novel targets for new cancer therapeutics. When applied to breast cancer, the method may lead to the identification of genes responsible for different subtypes of breast cancer, such as genes that mediate tamoxifen resistance, which in turn, may lead to the development of novel therapeutics to overcome tamoxifen resistance.

Provided are methods for identifying a ribonucleotide binding protein-associated biomarker, comprising the steps of (a) preparing a polysomal lysate from a cultured cell line, non-cultured cells, or solid tissue; (b) preparing a first immunoprecipitation complex from said polysomal lysate using an antibody directed against a ribonucleotide binding protein and a second immunoprecipitation complex from said polysomal lysate using an antibody which is an isotype of the antibody directed against the ribonucleotide binding protein; (c) extracting RNA from said immunoprecipitation complexes; (d) amplifying said RNA to form cDNA; (e) labeling said cDNA; (e) hybridizing said labeled cDNA to one or more nucleic acids immobilized on a microarray; and (f) determining the ratio of labeled cDNA prepared from the first immunoprecipitation complex to that obtained from the second immunoprecipitation complex bound to the one or more one or more nucleic acids immobilized on a microarray.

Also provided are ribonucleotide binding protein-associated biomarkers, which may be identified by any of the methods noted above, wherein the ratio of labeled cDNA prepared from the first immunoprecipitation complex to that obtained from the second immunoprecipitation complex bound to the one or more nucleic acids immobilized on a microarray is greater than 4, 6, 8, or 10.

Also provided are HuR-associated biomarkers. In some cases, the biomarkers are selected from the group consisting of CALM2, CD9, SRRM1, CCN1, DAZAP2, ARL6IP1, PTMA, ATP1B1, MMD and TMCO1, wherein the level of expression is over- or under-expressed in a breast cancer sample compared to a standard level of expression of the same biomarker in a non-cancerous sample.

In addition, a panel or set of biomarkers is provided comprising at least one HuR-associated biomarker selected from the group consisting of CALM2, CD9, SRRM1, CCN1, DAZAP2, ARL6IP1, PTMA, ATP1B1, MMD and TMCO1, wherein the level of expression is over- or under-expressed in a breast cancer sample compared to a standard level of expression of the same biomarker in a non-cancerous sample.

Also provided are methods of aiding in the identification of subjects at risk to develop breast cancer, aiding in the diagnosis of breast cancer, aiding in monitoring of disease status of breast cancer, aiding in the monitoring of breast cancer therapy, aiding in monitoring of breast cancer therapy safety, and aiding in the determination of the effectiveness of a chemotherapy agent in treating breast cancer.

A better understanding of the disclosed methods of identifying biomarkers, sets of biomarkers, and methods of using the sets to facilitate the diagnosis of, or to monitor the disease status or progression of a cancer, can be obtained from the following detailed descriptions and accompanying drawings, which set forth illustrative examples indicative of the various ways in which the principals of the disclosure may be employed.

### BRIEF DESCRIPTION OF THE DRAWINGS

The foregoing aspects and many of the advantages of this disclosure are more readily appreciated as the same become better understood by reference to the following detailed description, when taken in conjunction with the accompanying drawings, wherein:

FIG. 1 sets forth data illustrating Immunoprecipitation and RIP in MB-231 and MCF-7 breast cancer cells. Immunoprecipitations were performed from MB-231 or MCF-7 cell lysates using anti-HuR monoclonal antibody (3A2) and IgG1 isotype control. Panel 1A. IP Western of HuR revealed expected size band as detected by 3A2. The subpanel on the right reveals amounts of HuR in lysates used from both cell lines. Panel 1B. Verification by quantitative RT-PCR showed fifteen and eleven fold enrichments of B-ACTIN, a known HuR target, in the 3A2 IPs from MB231 and MCF-7, respectively. All ΔΔ C_{T} values were normalized to GAPDH. Experiments were done in duplicate (n=2).

FIG. 2 sets forth data illustrating that the HuR RIP-CHIP identifies distinct genetic profiles in ER+ and ER− breast cancer cells. HuR immunoprecipitations were performed from MB-231 or MCF-7 cell lysates using HuR antibody and IgG1 isotype control hybridized to Illumina Sentrix arrays (47,000 genes). Control signals were subtracted. Results represent cumulative data from 12 different arrays. Experiments were done in triplicate (n=3) for each cell line with matching controls. Scales are log 2.

FIG. 3 sets forth data illustrating the GO Classification of genes found by RIP CHIP of potential HuR targets and their relationship to the Acquired Capabilities of Cancer Model. Panel 3A. Differentially expressed genes which are more represented in the Biological Processes (BP) GO category than expected. Panel 3B. Original representation showing subsets of transcripts found to be targets of association with HuR (normal type). New transcripts found in this study with RIP-Chip (bold type). Enhanced expression upon binding to HuR influences several of the acquired capabilities of cancer cells described previously [Hanahan D, Weinberg R A, Cell 2000, 100(1):57-70; Lopez de Silanes I, Lal A, Gorospe M, RNA Biol 2005, 2(1):11-13].

FIG. 4 sets forth data illustrating validation of target CALM2 and CD9 mRNAs by quantitative RT-PCR. Quantitative RT-PCR using RNA extracted from cell lysates of RIP CHIP analysis confirmed results identifying CALM2 mRNA (A) and CD9 mRNA (B) as HuR targets. Change in gene expression is represented as fold increase in HuR immunoprecipitation as compared to IgG1. GAPDH mRNA was used as an endogenous control. Error bars represent SEM. p value is <0.005. Experiments were done in triplicate (n=3).

FIG. 5 sets forth data illustrating biotin pull-down assays of CD9 and CALM2. Panel 5A. Scheme of Coding region (CR) and 3′UTR fragments for biotin pull-down assay. The sequences were obtained from Entrez data base. CR and 3′UTR fragments selected for amplification by PCR are as noted. Panel 5B. 1% agarose gel electrophoresis showing PCR amplified products of the coding regions and 3′UTR's for CD9 (442 bp and 432 bp, respectively) and CALM2 (443 bp and 610 bp, respectively). Panel 5C. Biotin pull down assay using lysates prepared from MB-231 cells. The binding of HuR to biotinylated 3′UTR transcripts from both CD9 and CALM2 mRNAs was specific. HuR did not bind a biotinylated control (GAPDH 3′UTR) and did not bind to biotinylated transcripts spanning the CR of CD9 or CALM2. Experiments were done in duplicate (n=2).

FIG. 6 sets forth data illustrating that HuR differentially regulates CD9 and CALM2 in MB-231. Panel 6A. Epitope HA tagged HuR is over expressed by 142% and 138% respectively, in stably transfected clones 4E1 and 5F1, as compared to empty vector (EV) control clone 2C7. Panel 6B. HuR knock down using lentiviral short hairpin (sh) RNA H760 results in a 94% reduction in steady state levels of protein in clone A7 (LL=lentilox control). Panel 6C. HuR over expression results in a 40% reduction in CD9 protein levels as assayed by Western analysis; however, HuR knock down using lentiviral shRNA results in an increase from 100% to 228% of CD9 levels. Panel 6D. Over expression of HuR decreases CD9 mRNA levels but not CALM2 expression. Analysis of steady state CD9 and CALM2 mRNA levels by quantitative RT-PCR reveals significant decreases in CD9 mRNA levels, whereas CALM2 levels are unaffected. Although CALM2 expression appears greater, the change is not significant. Panel 6E. Knocking down HuR levels by shRNA in MB-231 cells shows significant increases in CD9 and CALM2 mRNA levels by quantitative RT-PCR. Decreased levels of HuR mRNA validate HuR shRNA knock down. Panel 6F. Graph showing the effects of HuR on the expression of CD9 mRNA. HuR over expression results in decreases in both mRNA and protein levels, though the decreases are greater in RNA. Whereas, HuR knock down by shRNA results in significant increases at both the mRNA and protein levels, with greater change at transcript levels. The dashed line represents levels in control cells. Error bars represent SEM. p value is <0.005; N.S.=not statistically significant; and *=statistically significant. All experiments were done in triplicate (n=3).

FIG. 7 sets forth data illustrating the effects of overexpressing or reducing HuR on CD9 and CALM2 expression in MCF-7 cells. Panel 7A. Western analysis of HuR over expression in heterogenous population of cells reveals approximately 10% over expression. Panel 7B. Lentiviral HuR shRNA efficiently knocks down HuR protein by over 90%. Panel 7C. HuR over expression and under expression results in small changes in CD9 protein levels in MCF-7 cells. Panel 7D. Levels of both CD9 and CALM2 mRNAs are unchanged in cells which over express HuR; whereas lentiviral knock down of HuR in MCF-7 cells results in decreases in steady-state mRNA levels (Panel 7E). The graph in Panel 7F shows minimal changes in CD9 mRNA and protein levels for in HuR over expressing MCF-7 cells. The CD9 mRNA levels, however, are more affected in HuR knock down. P value is <0.005; N.S.=not statistically significant; and *=statistically significant.

FIG. 8 sets forth data illustrating that total cellular levels of HuR are similar in MB-231 and MCF-7 cells. Nuclear and cytoplasmic separation was performed to measure levels of HuR in different compartments of MB-231 and MCF-7 cells. Total cellular HuR levels were very similar, whereas there was a small (10%) increase in HuR cytoplasmic levels in MB-231 cells as compared to MCF-7. Absence of tubulin staining demonstrates integrity of isolation as there should not be tubulin in the nuclear fraction. Bands were measured by densitometry and normalized to tubulin controls. (T=total cellular lysate; C=cytoplasmic lysate, N=nuclear lysate).

FIG. 9 sets forth data illustrating the relative baseline values of CALM1, CALM2, CALM3, and CD-9 mRNAs in ER+ and ER− cells. Quantitative RT-PCR performed on mRNA extracted from cell lysates showing relative levels of CALM1, CALM2, CALM3, and CD-9 mRNAs in MB231 and MCF-7 breast cancer cells. All values were normalized to GAPDH mRNA. All experiments were done in triplicate (n=3) except for CALM3 (n=2).

Abbreviations and their corresponding meanings include: aa or AA=amino acid; ER=estrogen receptor; mg=milligram(s); ml or mL=milliliter(s); mm=millimeter(s); mM=millimolar; nmol=nanomole(s); ORF=open reading frame; PCR=polymerase chain reaction; pmol=picomole(s); ppm=parts per million; RT=reverse transcriptase; RT=room temperature; SDS-PAGE=sodium dodecyl sulfate-polyacrylamide gel electrophoresis; U=units; ug, μg=micro gram(s); ul, μl=micro liter(s); and uM, μM micromolar; Estrogen receptor negative (ER−), estrogen receptor positive (ER+), RNA immunoprecipitation (RIP), RNA immunoprecipitation applied to microarrays (RIP-Chip), 3′ untranslated region (3′ UTR), ELAV1 (embryonic lethal abnormal vision 1).

The term “biomarker” in the context of the present invention encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological markers of health status, such as “clinical parameters” defined herein, as well as “traditional laboratory risk factors”, also defined herein. Biomarkers also include any calculated indices created mathematically or combinations of any one or more of the foregoing measurements, including temporal trends and differences.

The term “analyte” as used herein can mean any substance to be measured and can encompass electrolytes and elements, such as calcium.

The term “set” means a collection of objects, which may include zero, one, or two or more objects. A set of biomarkers, for example, includes a set of one biomarker, or more commonly, a set of two or more biomarkers.

### DETAILED DESCRIPTION

Provided are methods to identify novel biomarkers, which can be used for screening and diagnostic testing. The method may also identify novel targets for new cancer therapeutics. When applied to breast cancer tissues, the method may lead to the identification of genes responsible for different subtypes of breast cancer, such as genes that mediate tamoxifen resistance. Identification and characterization of similar genes may lead to the development of novel therapeutics that can overcome drug resistant forms of these and other types of cancer.

Presented is a method called Ribonomic Analysis, or RNA immunoprecipitations applied to microarrays (RIP-on-Chip), to identify en masse, in vivo targets of RBPs from cultured cell lines and solid tissues. RIP Chip technology can be used, for example, to identify cellular targets of HuR within cultured cell lines. The RIP-on-Chip was used to identify distinct subsets of HuR associated mRNAs in MDA MB231 and MCF-7 breast cancer cell lines. The role of HuR in triple negative breast cancer was also investigated by overexpressing HuR in MB231 cells, which results in accelerated growth. HuR pull down experiments demonstrated that the RIP-on-Chip technology can be used to identify known cancer targets, and distinct subsets of relevant cancer genes, plus novel cancer targets, suitable for detailed characterization.

One aspect of the invention relates to an HuR-associated biomarker selected from the group consisting of CALM2, CD9, SRRM1, CCN1, DAZAP2, ARL6IP1, PTMA, ATP1B1, MMD and TMCO1, wherein the level of expression is over- or under-expressed in a breast cancer sample compared to a standard level of expression of the same biomarker in a non-cancerous sample.

Another aspect of the invention relates to a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, wherein the level of expression at least one biomarker is over- or under-expressed in a breast cancer sample compared to a standard level of expression of the same biomarker in a non-cancerous sample.

A sample obtained from a subject, without being limiting, can include isolated cells, tissue samples, or bodily fluids, including blood, plasma, serum, sputum, urine, stools, tears, mucus, hair, skin, or other fluids secreted or excreted from various organs or specialized cells. Samples are typically obtained from humans, but may include tissues obtained from non-human primates or rodents. Samples may also include sections of tissues taken from biopsy or autopsy samples, explants and primary or transformed cell cultures derived from subject tissues. A sample can be compared on a cellular basis, or on a volume basis for fluids, or normalized to the amount of mRNA, protein, or other macromolecule or chemical in an extract obtained from a sample of dispersed cells, tissue, or biological fluid.

The standard level of expression, or a range of acceptable levels, of a biomarker can be determined by a variety of methods. For example, a reference range can be established by evaluating the distribution of said marker among non-cancerous samples obtained from a population of healthy subjects in conjunction with the corresponding distribution of cancerous samples. For biomarkers, values which exceed a critical threshold are of particular interest, so that only values outside the reference range in a particular direction are of use. The optimal threshold can be determined with respect to the most desired properties of the biomarker (e.g., sensitivity, specificity, reliability) which depends on the intended use (e.g., diagnostic or screening). Such methods for determining the optimal threshold include, receiver operating characteristic curves, Bayesian classifiers, or other decision-theoretic methods. In one aspect of the invention, it is measured as the median expression level of the biomarker in a non-cancerous sample obtained from one or more samples obtained from a population of healthy subjects. In another aspect of the invention, the standard level of expression is the median expression level of the biomarker in a non-cancerous sample obtained from one or more samples obtained from a subject having breast cancer.

Levels of protein expression may be determined by a number of techniques, as are well known to one of skill in the art. Examples include western blots, immunohistochemical staining and immunolocalization, immunofluorescence, enzyme-linked immunosorbent assay (ELISA), immunoprecipitation assays, agglutination reactions, radioimmunoassay, flow cytometry and equilibrium dialysis. These methods generally depend upon a reagent specific for identification of HuR associated-biomarkers. The reagent is may be an antibody and may comprise monoclonal or polyclonal antibodies. Fragments and derivatized antibodies may also be utilized, to include without limitation Fab fragments, ScFv, single domain antibodies, nanoantibodies, heavy chain antibodies etc which retain binding function. Any detection method may be employed in accordance with the invention. The nature of the reagent is not limited except, that it must be capable of specifically identifying HuR associated-biomarkers.

Suitable methods for determining HuR associated-biomarkers expression at the RNA level are well known in the art. Methods employing nucleic acid probe hybridization to the HuR associated-biomarkers transcript may be employed for measuring the presence and/or level of HuR associated-biomarkers mRNA. Such methods include use of nucleic acid probe arrays (microarray technology) and Northern blots. Advances in genomic technologies now permit the simultaneous analysis of thousands of genes, although many are based on the same concept of specific probe-target hybridization.

Sequencing-based methods are an alternative. These methods started with the use of expressed sequence tags (ESTs), and now include methods based on short tags, such as serial analysis of gene expression (SAGE) and massively parallel signature sequencing (MPSS). Differential display techniques provide yet another means of analyzing gene expression; this family of techniques is based on random amplification of cDNA fragments generated by restriction digestion, and bands that differ between two tissues identify cDNAs of interest.

In one aspect, the levels of HuR associated-biomarkers gene expression are determined using reverse transcriptase polymerase chain reaction (RT-PCR). RT-PCR is a well known technique in the art which relies upon the enzyme reverse transcriptase to reverse transcribe mRNA to form cDNA, which can then be amplified in a standard PCR reaction. Protocols and kits for carrying out RT-PCR are extremely well known to those of skill in the art and are commercially available.

In another aspect, the RT-PCR is carried out in real time and in a quantitative manner. Real time quantitative RT-PCR has been thoroughly described in the literature (see Gibson et al for an early example of the technique) and a variety of techniques are possible. Examples include use of Taqman, Molecular Beacons, LightCycler (Roche), Scorpion and Amplifluour systems. All of these systems are commercially available and well characterised, and may allow multiplexing (that is, the determination of expression of multiple genes in a single sample).

These techniques produce a fluorescent read-out that can be continuously monitored. Real-time techniques are advantageous because they keep the reaction in a “single tube”. This means there is no need for downstream analysis in order to obtain results, leading to more rapidly obtained results. Furthermore, keeping the reaction in a “single tube” environment reduces the risk of cross contamination and allows a quantitative output from the methods of the disclosure.

Variants on the basic PCR technique may also be used such as nested PCR, equivalents may also be included within the scope of the invention. Examples include isothermal amplification techniques such as NASBA, 3SR, TMA and triamplification, all of which are well known in the art and commercially available. Other suitable amplification methods include the ligase chain reaction (LCR) [Barringer et al, Gene 1990, 89(1):117-122], selective amplification of target polynucleotide sequences [U.S. Pat. No. 6,410,276], consensus sequence primed polymerase chain reaction [U.S. Pat. No 4,437,975], arbitrarily primed polymerase chain reaction [WO 90/06995] and nick displacement amplification [WO 2004/067726].

The panel or set comprises at least one, two, three, four, etc of the HuR associated-biomarkers genes listed, up to all genes. All permutations and combinations of the genes listed above are contemplated for gene panels.

For larger panels, use of microarrays may be used in determining levels of HuR associated-biomarkers indirectly by looking at expression of other genes, comprising probes immobilized on a solid support hybridizing with transcripts or parts thereof of at least one gene selected from those listed. For these groups of genes the changes in expression are calculated to be highly significant (p<0.01). The probes may be immobilized on a solid support hybridizing with transcripts or parts thereof of at least one, two, three, four, etc of the genes listed above, up to all of the genes. All permutations and combinations of the genes listed above are contemplated within the scope of the present invention, for the purposes of providing a microarray. Microarrays and their means of manufacture are well known and can be manufactured to order by commercial entities, such as Agilent and Affymetrix.

The elements of probe selection and design are common to the production of all arrays, regardless of their intended application and as such would be well known to one of skill in the art. Strategies to optimize probe hybridization, for example, may be included in the process of probe selection. Hybridization under particular pH, salt, and temperature conditions can be optimized by taking into account melting temperatures and using empirical rules that correlate with desired hybridization behaviors.

To facilitate comparisons, the level of expression in a biomarker in breast cancer sample, is measured against a similar, if not identical, amount of sample used to determine the standard level of expression the biomarker in a non-cancerous sample obtained from a population of subjects, or from a non-cancerous sample obtained from a subject having breast cancer. The standard can be measured, if desired, from the same subject from whom the breast cancer sample was obtained.

In one aspect, the ratio of expression of at least one biomarker expressed in the breast cancer sample compared to the non-cancerous sample is less than ½ or greater than 2. Higher and lower thresholds can be used, such as less than ¼ and greater than 4, less than ⅛ and greater than 8, less than 1/16 and greater than 16, etc., to facilitate statistical analysis, where adequate to optimal signal-to-noise ratios are used for each of the biomarkers in the set of biomarkers used to aid in the diagnosis of, or to monitor the disease status or progression of, breast cancer in a subject.

In one aspect of the invention, the breast cancer is an estrogen receptor positive breast cancer. In another aspect of the invention, the breast cancer is an estrogen receptor negative breast cancer.

In one aspect of the invention, at least one of said biomarkers is an mRNA. In another aspect of the invention, at least one of said biomarkers is a polypeptide. In another aspect of the invention, at least one of said biomarkers is post-transcriptionally regulated. The set of biomarkers may include biomarkers that are all based on mRNAs, or biomarkers that are all based on polypeptides. The set may also encompass a mix of both mRNA- and polypeptide-based biomarkers.

In one aspect, the set of biomarkers may further comprise at least one biomarker selected from the group consisting of Prothymosin-α, Bcl-2, Mcl-1, SirT1, TGF-b, MMP-9, MTC-1, μPA, VEGF-α, HIF1-α and cyclins A1 (CCNA1), B1 and D1.

In another aspect, the set of biomarkers may further comprise at least one biomarker selected from the group consisting of Glut-1, ERα, COX-2, IL-8, Cyclin E1, BRCA-1 and Thrombospondin 1.

In another aspect, the set of biomarkers may further comprise at least one biomarker selected from the group consisting of CD9, PTMA, UBE2E2, CCNI, CKLF, SRRM1, STK4, FKBP1A, PMP22, CALM2, MMD, CSDA, CHIC2, DAZAP2, ZNF22, ATP1B1, TRAM1, ENY2, ALKBH5, RAP2A, TMCO1, and ARL6IP1.

In another aspect, the set of biomarkers may further comprise at least one biomarker selected from the group consisting of ACTB, SMNDC1, MAL2, CALM2, CDK2AP1, hCG_{—}1781062, JUND, ARL6IP1, PTMA, ATP6V1G1, ACTB, HMGB1, BUB3, PJA2, LOC203547, NPM1, MATR3, TMC01, CXCR7, ZFP36L1, SFRS2, TMSL3, PLOD2, PPP6C, EIF4A2, RPS6KB1, HSPA1A, TIMEM59, FOXA1, PEX11B, MYB, CD9, ZNF14, ITGB1, PARD6B, LOC441087, SRRM1, SNX16, PUM1, MORF4L1, TFDP1, MMD, GCA, CISD2, C4orf34, DAZAP2, G3BP1, C21orf55, NCOA3, ATP1B1, SFPQ, PRKAR1A, YBX1, HIST1H3E, CCNI, CSTB, C15orf51, YWHAZ, PRIM2, SLC7A1, C15orf15, PCBP2, ROD1, SPINT2, CALMG, and YTHDC1.

One aspect is directed to a kit for measuring the level of expression of a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, wherein the level of expression at least one biomarker is over- or under-expressed in a breast cancer sample compared to a standard level of expression of the same biomarker in a non-cancerous sample.

Kits for use in diagnostic, research, and therapeutic applications may include any or all of the following items: assay reagents, buffers, hybridization probes or primers, biomarker-specific nucleic acids or antibodies, antisense polynucleotides, siRNAs, shRNAs, ribozymes, small molecule inhibitors of cancer-associated enzymes or nucleic acids, reaction tubes, etc. Kits intended for therapeutic use may include sterile saline or other pharmaceutically-acceptable solutions. Kits may also include instructional materials, which may be written or encoded on electronic storage media. A wide variety of kits and components may be prepared according to the present invention, depending on its intended use. Kits of the invention typically be used to evaluate a plurality of genes or gene products which are selected based on statistically significant parameters relating to the diagnosis, diseases status, or progression of a disease of interest.

The kit may also include reagents necessary for a nucleic acid amplification step. Reagents may include, by way of example and not limitation, amplification enzymes, probes, positive control amplification templates, reaction buffers etc. For example, in the PCR method of amplification, possible reagents include a suitable polymerase such as Taq polymerase and appropriate PCR buffers, and in the TMA method the appropriate reagents include RNA polymerase and reverse transcriptase enzymes. All of these reagents are commercially available and well known in the art.

The kit may further include components required for real time detection of amplification products, such as fluorescent probes for example. The relevant real-time technologies, and the reagents required for such methods, are well known in the art and are commercially available. Probes may need to be of sequence such that they can bind between PCR primer sites on the nucleic acid molecule of interest that is subsequently detected in real-time. Other probes may be designed that bind to a relevant portion of the relevant nucleic acid sequence. Suitable probes are accordingly included in a further aspect of the kits of the invention. Kits for use in methods where recruitment to a promoter, or levels of histone acetylation are measured, may include suitable components necessary for carrying out a chromatin immunoprecipitation.

Once the level or activity of HuR associated-biomarkers has been determined, it is then possible to conclude which type of treatment is suitable or not. Accordingly, a suitable information sheet may be incorporated in the kit which allows the user of the kit to interpret the results to thus decide on an appropriate course of treatment. The sheet may take the form of written instructions, or a flow chart or decision tree, for example.

One aspect is directed to a method for aiding in the diagnosis of breast cancer in a subject comprising: (a) obtaining a sample from said subject; (b) measuring the level of expression of a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, in said sample obtained from the subject; and (c) comparing the level of expression of each biomarker in the set of HuR-associated biomarkers to the standard level of expression of the same biomarker in a non-cancerous sample; wherein a significant difference in the ratio of expression of at least one biomarker in the set aids in the diagnosis of breast cancer.

Another aspect of the invention is directed to a method for monitoring the disease status or progression of breast cancer in a subject comprising: (a) obtaining a sample from said subject; (b) measuring the level of expression of a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, in said sample obtained from the subject; and (c) comparing the level of expression of each biomarker in the set of HuR-associated biomarkers to the standard level of expression of the same biomarker in a non-cancerous sample; wherein a significant difference in the ratio of expression of at least one biomarker in the set aids in monitoring the disease status or progression of breast cancer.

Another aspect of the invention is directed to a method for monitoring the disease status of breast cancer in a subject comprising: (a) obtaining a sample from said subject; (b) measuring the level of expression of a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, in said sample obtained from the subject; and (c) comparing the level of expression of each biomarker in the set to the standard level of expression of each corresponding biomarker in the set in a non-cancerous sample; wherein a significant difference in the ratio of expression of at least one biomarker in the set aids the disease status of breast cancer in a subject. It is appreciated that this method can be performed multiple times on multiple samples and that the comparison of biomarker levels from one time to the next can be important in determining the status of the disease. For instance, this method may be performed before anti-cancer treatment, during treatment and/or after treatment as a means of measuring the response of the subject to the treatment. In addition, the method may be applied longitudinally to a subject without any anti-cancer treatment to monitor disease status.

In one aspect, the ratio of expression of at least one biomarker expressed in the breast cancer sample compared to the non-cancerous sample is less than ½ or greater than 2. Higher and lower thresholds can be used, such as less than ¼ and greater than 4, less than ⅛ and greater than 8, less than 1/16 and greater than 16, etc., to facilitate the statistical analysis, where adequate to optimal signal-to-noise ratios are used for each of the biomarkers in the set of biomarkers used to aid in the diagnosis or to monitor the disease status or progression of breast cancer in a subject.

Also provided is a method of identifying a ribonucleotide binding protein associated biomarker, comprising the steps of (a) preparing a polysomal lysate from a cultured cell line, non-cultured cells, or solid tissue; (b) preparing a first immunoprecipitation complex from said polysomal lysate using an antibody directed against a ribonucleotide binding protein and a second immunoprecipitation complex from said polysomal lysate using an antibody which is an isotype control of the antibody directed against the ribonucleotide binding protein; (c) extracting RNA from said immunoprecipitation complexes; (d) amplifying said RNA to form cDNA; (e) labeling said cDNA; (e) hybridizing said labeled cDNA to one or more nucleic acids immobilized on a microarray; and (f) determining the ratio of labeled cDNA prepared from the first immunoprecipitation complex to that obtained from the second immunoprecipitation complex bound to the one or more one or more nucleic acids immobilized on a microarray.

In this aspect, the biomarker is a cancer biomarker. The biomarker may be a ribonucleotide binding protein-associated biomarker. The biomarker may be a breast cancer biomarker, which may be estrogen receptor positive, or estrogen receptor negative. Also provided are ribonucleotide binding protein associated biomarkers which may be identified by the method of noted above, wherein the ratio of labeled cDNA prepared from the first immunoprecipitation complex to that obtained from the second immunoprecipitation complex bound to the one or more nucleic acids immobilized on a microarray is at least greater than 2. In other aspects, the ratio is at least greater than 4, the ratio is at least greater than 5, the ratio is at least greater than 6, the ratio is at least greater than 8, or the ratio is at least greater than 10.

While specific examples have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope, which is to be given the full breadth of the appended claims and any equivalent thereof.

### EXAMPLES

The foregoing discussion may be better understood in connection with the following representative examples which are presented for purposes of illustrating the principle methods and compositions of the invention and not by way of limitation. Various other examples will be apparent to the person skilled in the art after reading the present disclosure without departing from the spirit and scope of the disclosure. It is intended that all such other examples be included within the scope of the appended claims.

All parts are by weight (e.g., % w/w), and temperatures are in degrees centigrade (° C.), unless otherwise indicated.

### Cell Culture Methods

The MDA-MB-231 (MB-231) and MCF-7 cell lines were obtained from American Type Culture Collection (Manassas, Va.). The cell lines were maintained at 37° C. in a humidified atmosphere of 95% air and 5% CO₂. MB-231 cells were grown in RPMI (GIBCO®, Invitrogen™, Carlsbad, Calif.) containing 10% fetal calf serum (Hyclone, Thermo Fisher Scientific, Waltham, Mass.), 0.5 mM L-glutamine (GIBCO®), 25 mg/ml glucose (Sigma-Aldrich), HEPES (GIBCO®) and Sodium Pyruvate (GIBCO®). MCF-7 cells were grown in DMEM (GIBCO®) supplemented with 10% fetal calf serum.

### HuR Immunoprecipitations (RIP-Chip)

HuR RIP-Chip analysis was performed as previously described [Intine R V et al., Mol Cell 2003, 12(5):1301-130; Atasoy U et al., J Immunol 2003, 171(8):4369-4378; Casolaro V et al., The Journal of Allergy and Clinical Immunology 2008, 121(4):853-859 e854]. Briefly, lysates were prepared from exponentially growing MB-231 and MCF-7 cells. Equal amounts of protein lysates were used (100-300 μg). HuR monoclonal antibody 3A2 (made in our laboratory from the 3A2 hybridoma, generously provided by Dr. Joan Steitz, Yale University, New Haven, Conn.) or isotype control IgG1 (BD Biosciences, San Jose, Calif.) were pre-coated onto protein A Sepharose beads (PAS) and extensively washed. Lysates from each cell initially were pre-absorbed with 30 μg of IgG1, and then removed by addition of PAS beads. Individual pull down assays were performed at 4° C. for only 1-2 hr to minimize potential re-assortment of mRNAs.

### RNA Amplification

The entire amount of recovered RNA per immunoprecipitation was amplified using the WT-Ovation™ Pico RNA Amplification System protocol (NuGen, San Carlos, Calif.). Forty ng of total RNA was used as starting material to generate at least 6 μg of cDNA. Amplified cDNA was purified using Zymo Research Clean and Concentrator™-25 (Zymo Research, Orange, Calif.). Three μg of amplified and purified cDNA was incubated at 50° C. for 30 minutes with 5 μl of UNG buffer and 5 μl UNG enzyme and 60 minutes with 5 μl labeling buffer and 5 μl ARP (biotin) solution as described in NuGen's labeling protocol for the Illumina Beadarray platform. All samples (total RNA, amplified cDNA, and biotin labeled amplified cDNA) were quantitated using a Nanodrop™ (Thermo Fisher Scientific, Waltham, Mass.) spectrophotometer. RNA quality and integrity were assessed on selected samples with the Experion™ automated electrophoresis system (Bio-Rad, Hercules, Calif.).

### Microarray

Biotin-labeled, amplified cDNA (1.5 μg) was hybridized to a Sentrix® Human-6 v.2 Whole Genome Expression BeadChips (Sentrix Human WG-6; Illumina, San Diego, Calif.). Each chip tested 6 samples and contained 47,293 gene targets, representing 18,025 distinct RefSeq genes that are not pseudogenes. A total of 3 chips were used for this experiment. The chips were hybridized at 48° C. for 20 hr in the hybridization buffer provided by the manufacturer. After hybridization, the chips were washed and stained with streptavidin-C3. The chips were scanned on the BeadArray Reader, as described by Illumina. The Illumina Beadstudio software was used to assess fluorescent hybridization signals.

### Quantitative RT-PCR

Selected genes were validated by quantitative RT-PCR. Briefly, cDNA was generated from the same samples as previously described for the microarray experiments using 10 ng total RNA and the SuperScript™ III Platinum® Two-Step qRT-PCR Kit with SYBR® Green (Invitrogen Carlsbad, Calif.). RT-PCR was performed on the StepOne™ Real-Time PCR System (Applied Biosystems, Foster City, Calif.). Each sample was run in triplicate for these genes and the cDNA was divided equally per reaction in a 20 μl volume. The PCR conditions were: 50° C. for 2 minutes and 95° C. for 2 minutes, followed by 40 cycles of 95° C. for 15 seconds alternating with 60° C. for 30 seconds. Melting curve analysis was performed on every reaction to confirm a single amplicon. For each cell line, differences in gene expression were determined using the equation 2^{-ΔΔCt}, where the Cₜ value for either the HuR or IgG IP was subtracted from the Cₜ value of the GAPDH control. For each cell line, the ΔCₜ value for the HuR and IgG IP were computed in triplicate and averaged to give one ΔΔCₜ value per sample. Primers used:

Human RT GAPDH
(SEQ ID NO: 1)
Forward 5′ AGCCTCAAGATCATCAGCAATGCC 3′
(SEQ ID NO: 2)
Reverse 5′ TGTGGTCATGAGTCCTTCCACGAT 3′
Mouse RT HuR
(SEQ ID NO: 3)
Forward 5′ ACTGCAGGGATGACATTGGGAGAA 3′
(SEQ ID NO: 4)
Reverse 5′ AAGCTTTGCAGATTCAACCTCGCC 3′
Human RT HuR
(SEQ ID NO: 5)
Forward 5′ ATGAAGACCACATGGCCGAAGACT 3′
(SEQ ID NO: 6)
Reverse 5′ AGTTCACAAAGCCATAGCCCAAGC 3′
Human RT CD9
(SEQ ID NO: 7)
Forward 5′ TCAGACCAAGAGCATCTTCGAGCA 3′
(SEQ ID NO: 8)
Reverse 5′ ACCAAGAGGAAGCCGAAGAACAGT 3′
Human RT CALM2
(SEQ ID NO: 9)
Forward 5′ CTGACCAACTGACTGAAGAGCAGA 3′
(SEQ ID NO: 10)
Reverse 5′ TTCTGTGGGATTCTGCCCAAGAG 3′

### Cloning Strategy of HA HuR

A hemagglutinin (HA)-tagged human HuR gene [Gubin M M et al., Cell Cycle 2010, 9(16):3337-46] was cloned into the NheI and XhoI sites of the pZeoSV2(−) vector (Invitrogen). The plasmids were sequenced in both directions to confirm identity. Cells were transfected with either pZeo HA HuR or pZeo empty vector using Lipofectamine 2000 (Invitrogen). After five days, the transfected media was removed, and replaced with fresh medium containing 200 μg/ml of Zeocin antibiotic (Invitrogen). Cells were selected for a ten day period. After ten days, the selected cells were maintained in 50 μg/ml of Zeocin to maintain pZeo HA HuR and the empty expression vector control. No viable cells remained in the untransfected well. Cells were then cloned by limiting dilution.

### Lentiviral RNAi HuR Knockdown

To establish lentivirus to knockdown HuR, PSICOOLIGOMAKER v1.5 software (web.mit.edu/ccr/labs/jacks) was used to identify optimal shRNAs sequences to HuR. We tested multiple sequences, and chose the following sequence, designated shRNA H760, for further study:

shRNA H760
(SEQ ID NO: 11)
5′-GGATCCTCTGGCAGATGT-3′

Sense and anti-sense DNAs (prepared by Integrated DNA Technologies, Inc, IDT, Coralville, Iowa) were annealed to form a duplex DNAs with stem loops and a hairpin, that were cloned into in the Lentilox pII3.7 vector (ATCC) between the HpaI and XhoI restriction sites.

The DNA inserts were verified by sequencing, and the resulting lentiviral DNAs were packaged in 293FT cells using a ViraPower Lentiviral Expression Systems kit (Invitrogen) according to the protocol provided by the manufacturer. MB-231 and MCF-7 cells were both seeded at a density of 100,000 cells in 100 mm tissue culture plates with 10 ml of media. The following day, lentiviruses expressing either GFP and no shRNA (empty lentilox control) or GFP and HuR shRNA H760, were added at a multiplicity of infection (MOI) of 10, along with polybrene (8 μg/ml) (Sigma-Aldrich Corp, St. Louis, Mo.). After five days, cells were harvested by trypsinization and sorted for GFP expression using BD FACSDiva cell sorter (BD Bioscience). Cells were cloned by limiting dilution and GFP expression was assessed using FACScan (BD Bioscience) and Cell Quest software (BD Bioscience). GFP expression was >98%, indicating a homogenous cell population.

### SDS-PAGE and Western Blot Analysis

Western analysis was performed as described previously, with slight modifications [Atasoy U et al., J Immunol 2003, 171(8):4369-4378]. Briefly, cells were harvested and lysed in triple-detergent RIPA buffer, with a protease inhibitor cocktail (Roche, Pleasanton, Calif.). For nuclear and cytoplasmic fractionations, the NE-PER kit was used (Pierce, Rockford, Ill.). Protein quantity was determined by Bradford Assay. Forty μg samples of protein were separated by electrophoresis on a 12% SDS-polyacrylamide gel and transferred to a nitrocellulose membrane. The membrane was blocked with 5% nonfat milk powder at room temperature for 1 hr and incubated with anti-β-tubulin (1 μg/ml, Sigma-Aldrich) at 4° C. overnight. After washing, the membrane was incubated with monoclonal anti-HuR clone 3A2 antibody (1 μg/ml) at room temperature for 1 hr or anti-CD9 antibody (1:100) (Santa Cruz Biotechnology, Inc., Santa Cruz, Calif.) at 4° C. overnight. The secondary antibody, a sheep anti-mouse Ig horse radish peroxidase (1:4000) (GE Healthcare, Piscataway, N.J.), was used with an incubation period of 1 hour at room temperature. Specific proteins were detected using chemiluminescence (GE Healthcare). HuR knockdown was determined to be >90% using Bio-Rad's Quantity One software (Bio-Rad) normalizing to β-tubulin, and HuR overexpression was quantitated in a similar manner.

### Biotin Pull-Downs

Biotinylated transcripts were synthesized using cDNA that was prepared from MB-231 cells. Templates were prepared using forward primers that contained the following T7 RNA polymerase promoter sequence:

[T7]
(SEQ ID NO: 12)
CCAAGCTTCTAATACGACTCACTTATAGGGAGA

Primers used for the preparation of biotinylated transcripts spanning the CD9 CR, and 3′UTR (NM_{—}001769) and CALM2 CR and 3′UTR (NM_{—}001743.3) were as follows:

CD9 CR 118-560: [T7]
(SEQ ID NO: 13)
5′ TCAAAGGAGGCACCAAGTGCAT 3′
and
(SEQ ID NO: 14)
5′ AACGCATAGTGGATGGCTTTCA 3′
CD9 3′UTR 798-1231: [T7]
(SEQ ID NO: 15)
5′ AGTCAGCTTACATCCCTGAGCA 3′
and
(SEQ ID NO: 16)
5′ GACATTGTCATAATTTTTTATTATGTATC 3′
CALM2 CR 72-515: [T7]
(SEQ ID NO: 17)
5′ GCTGACCAACTGACTGAAGA 3′
and
(SEQ ID NO: 18)
5′ CTTTGCTGTCATCATTTGTACAAA 3′
CALM2 3′UTR 518-1128: [T7]
(SEQ ID NO: 19)
5′ AGACCTTGTACAGAATGTGTTAA 3′
and
(SEQ ID NO: 20)
5′ GGGTAAATTGTAATTTTTTTATTGGAA 3′
GAPDH 3′UTR: [T7]
(SEQ ID NO: 21)
5′ CCTCAACGACCACTTTGTCA 3′
and
(SEQ ID NO: 22)
5′ GGTTGAGCACAGGG TACTTTATT 3′

The PCR-amplified fragments were purified and used as templates for in vitro synthesis of the corresponding biotinylated RNAs using a MAXIscript kit (Ambion®, Applied Biosystems). Biotin pull-down assays were performed by incubating 40 μg of MB-231 cell lysates with equimolar amounts of biotinylated transcripts for 1 hr at room temperature. The complexes were isolated using paramagnetic streptavidin-conjugated Dynabeads (Dynal®, Invitrogen), and the bound proteins in the pull-down material were analyzed by Western blotting using an antibody recognizing HuR (Santa Cruz). After secondary-antibody incubations, the signals were visualized by chemiluminescence (Amersham Biosciences, GE Healthcare).

### Statistical Analysis of Microarray Data

Analysis of microarray gene expression data was primarily performed using the Linear Models for Microarray Data (limma) package [Smyth G, In. Edited by Gentleman R C V, Dudoit S, Irizarry R, Huber W. New York: Springer; 2005] and the lumi package [Du P et al., Bioinformatics 2008, 24(13):1547-1548], available through the Bioconductor project [Gentleman R C et al., Genome Biol 2004, 5(10):R80] for use with R statistical software [Team R D C, In: ISBN 3-900051-07-0. vol. http://www.r-project.org: R Foundation for Statistical Computing Vienna, Austria; 2006]. After data pre-processing was completed the statistical analysis was performed using moderated t-statistics applied to the log-transformed (base 2) normalized intensity for each gene using an Empirical Bayes approach [Smyth G K, Stat Appl Genet Mol Biol 2004, 3:Article 3]. Three contrasts of interest were computed and tested. The first was the difference between HuR pulldown and IgG background for the MB-231 cell line. Genes which exhibited significantly greater expression in the pull-down assays were considered to be in the HuR pellet for the MB-231 cell line. The second contrast was similar to the first, but for the MCF-7 cell line. The third and most important contrast, was the difference between the first and second contrast, and can be viewed as a test of statistical interaction between HuR and the cell line. For a given gene, this term can be interpreted as reflection of the synergistic relationship between HuR and estrogen in breast cancer. Adjustment for multiple testing was made using the false discovery rate (FDR) method of Benjamini and Hochberg [Journal of the Royal Statistical Society 1995, Series B 57:289-300] with an FDR of 10% as our cutoff for declaring significance. To facilitate interpretation, log-fold-changes were transformed back to fold-changes on the data.

Gene ontology (GO) analyses were carried out on the list of significant genes based on the third contrast described above. The purpose of the analyses was to test the association between Gene Ontology Consortium categories [Consortium T G O, Nat Genetics 2000, 25:25-29] and differentially-expressed HuR pellet genes between MB-231 and MCF-7. Using our defined gene universe, GOstats [Falcon S, Gentleman R, Bioinformatics 2007, 23(2):257-258] was used to carry out conditional hypergeometric tests. These tests exploit the hierarchical nature of the relationships among the GO terms for conditioning [Alexa A et al., Bioinformatics 2006, 22:1600-1607]. We carried out GO analyses for over-representation of biological process (BP), molecular function (MF), and cellular component (CC) ontologies, and computed the nominal hypergeometric probability for each GO category. These results were used to assess whether the number of selected genes associated with a given term was larger than expected, and a p-value cutoff of 0.01 was used. GO categories containing less than 10 genes from our gene universe were not considered to be reliable indicators, and are not reported.

### Microarray Data Preprocessing

Data quality was examined by looking at quality controls metrics produced by Illumina's software (BeadStudio v3.1.3.0, Gene Expression Module 3.2.7). The data were then exported for further analyses. R. Image plots of each array were examined for spatial artifacts, and there was no evidence of systematic effects indicative of technical problems with the arrays. Within limma, quantile normalization was used for between chip normalization. Finally, quality control statistics were computed using a variety of Illumina's internal control probes that are replicated on each array. Any probes which were considered “not detectable” across all samples were excluded from further statistical analyses in order to reduce false positives. The determination of “not detectable” was based upon the BeadStudio computed detection p-value being greater than 1%.

### Gene Ontology Gene Universe

In defining the gene universe for the analysis, non-specific filtering was used to increase the statistical power without biasing the results. We started with all probes on the Illumina array which had both an Entrez gene identifier [Maglott D et al., Nucleic Acids Res 2005, 33(Database issue):D54-58] and a GO annotation, as provided in the lumiHumanAll.db [Du P et al., R package version 1.12.0] annotation data package and GO.db [Carlson M et al., R package version 2.4.5] annotation maps (built using data obtained from NCBI on Apr. 2, 2008). This set was then reduced by excluding probes that exhibited little variability (interquartile range (IQR) of <0.1 on log₂ scale) across all samples because such probes are generally not informative. Finally, for probes that mapped to the same Entrez identifier, a single probe was chosen in order to insure a subjective map from probe IDs to GO categories (via Entrez identifiers). This was necessary to avoid redundantly counting GO categories which produces false positives. Probes with the largest IQR were chosen to be associated with an Entrez identifier.

### Example 1

### Identification of Genes in Estrogen Receptor-Positive (ER+) and Estrogen Receptor-Negative (ER−) Breast Cancer Cell Lines Using RIP Chips

Distinct subsets of RNP-associated mRNAs in two breast cancer cell lines, MDA MB231 estrogen receptor negative (ER−) and MCF-7 estrogen receptor positive (ER+) were identified using a modified protocol using RIP chips, as described below. Briefly, method includes the steps of (1) preparing polysomal lysates; (2) performing immunoprecipitation with RBP antibodies; (3) extracting RNA; (4) amplifying and labeling recovered RNA, and (5) hybridizing to genome-wide microarrays.

The technology can also be used to facilitate the identification and characterization of well known and novel genes targeted by ribonucleotide binding proteins, including genes involved in the regulation of cancer and related metabolic pathways.

### HuR Immunoprecipitations from ER+ and ER− Breast Cancer Cell Lines

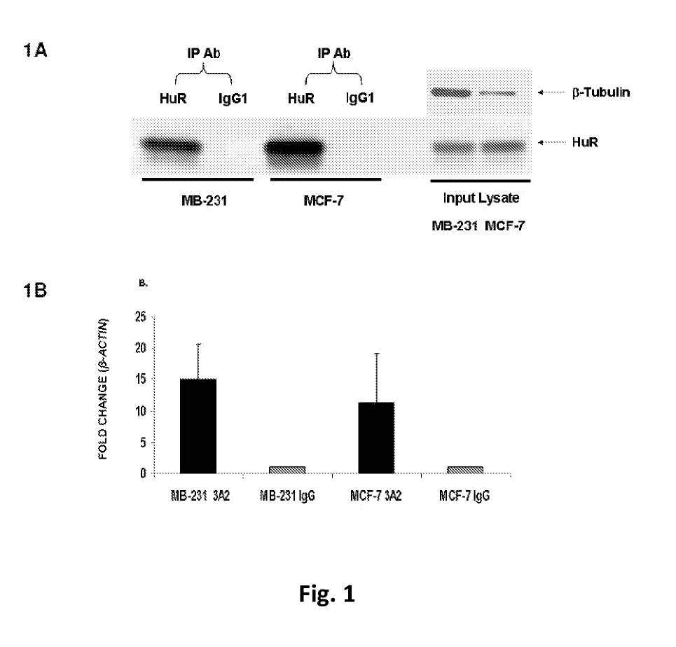

We first determined HuR protein expression levels in breast cancer cell lines. HuR is expressed in both the ER− and the ER+ cell lines, MB-231 and MCF-7, respectively (FIG. 1A). RNA immunoprecipitations, using HuR monoclonal antibody 3A2, recovered HuR (FIG. 1A) and revealed, by quantitative RT-PCR, a significant enrichment of up to fifteen fold for a known HuR target, B-ACTIN mRNA, as compared to isotype control (IgG1) and normalized to a non-target, GAPDH mRNA (FIG. 1B). These data showed that HuR RIP specifically immunoprecipitate HuR protein and associated mRNAs, though absolute quantitative conclusions cannot be drawn since different amounts of lysates were used and efficiency of immunoprecipitation from different cell lines may differ.

### RIP-Chip from ER+ and ER− Breast Cancer Cell Lines Identifies Unique Sets of Associated mRNAs

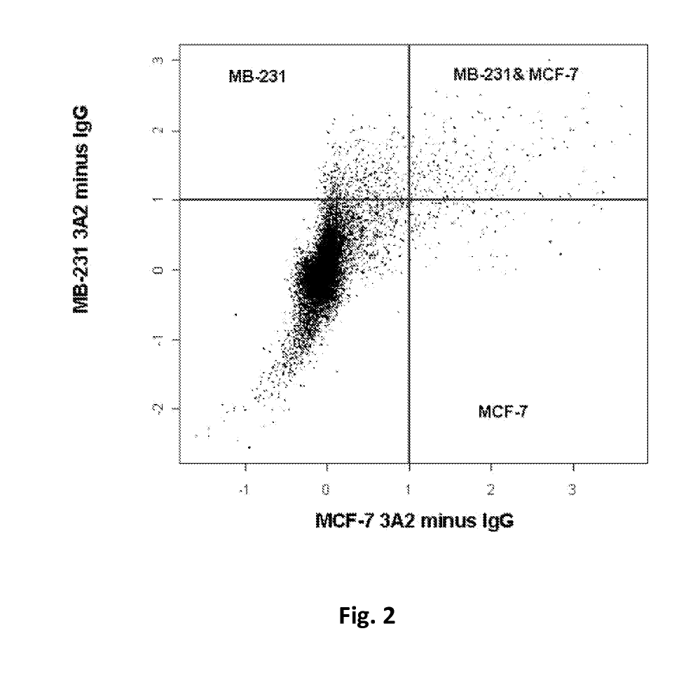

RIP-Chip was performed on cytoplasmic lysates from both breast cancer cell lines with HuR antibody and isotype control in order to determine HuR associated mRNAs. Each immunoprecipitation was done at least three independent times with matching controls. Signals from isotype control were subtracted out. Recovered mRNA was amplified and hybridized to Illumina Sentrix Human arrays consisting of 47,000 genes. FIG. 2 represents a composite array generated by combining hybridizations to twelve different arrays (log 2 scale). Three groups of HuR-associated target genes were identified: MB-231 targets in the left upper quadrant; both MB-231 and MCF-7 targets in the right upper quadrant; MCF-7 targets in the right lower quadrant. As expected, most of the mRNAs did not associate with HuR and were located in the lower left quadrant. There were 395 and 64 annotated genes, at least 2-fold or more enriched, associated with either MB-231 or MCF-7 cells, respectively, and 182 genes associated with both cell lines. A complete list can be found in Table 1.

TABLE 1
Complete GO analysis: Listing of HuR-associated genes with odds ratios and functional categories.

| | | | | | | | |
|---|---|---|---|---|---|---|---|
| | P | Odds | Exp | | | | |
| GOID | value | Ratio | Count | Count | Size | Term | Genes |

Molecular Function

| | | | | | | | |
|---|---|---|---|---|---|---|---|
| GO: 0005515 | 0 | 1.88 | 64.14 | 83 | 3798 | protein binding | NAMPT^{2.17}, SPRY1^{2.53}, SSSCA1^{0.46}, RAD51AP1^{2.01}, FRS3^{2.69}, |
| | | | | | | | TMED2^{2.8}, KDELR2^{2.21}, FOXN3^{2.62}, FBXO27^{2.03}, DCBLD2^{2.54}, |
| | | | | | | | LAYN^{2.25}, E2F7^{2.24}, CSNK1E^{4.06}, CSNK2B^{2.7}, CSTF3^{2.09}, |
| | | | | | | | CNKSR3^{2.48}, KIAA1949^{2.2}, DPYSL2^{2.76}, KCTD6^{2.57}, EREG^{2.86}, |
| | | | | | | | ERH^{2.07}, PHLDA1^{2.57}, SYNE1^{2.02}, KIAA0999^{2.01}, COTL1^{3.84}, |
| | | | | | | | GATA3^{0.28}, GJA1^{2.06}, LSM1^{2.1}, KIAA1267^{2.16}, CNOT7^{2.29}, |
| | | | | | | | HSPA1A^{0.34}, IFNGR2^{2.94}, IL8^{2.87}, ITGAE^{2.34}, ACAT1^{2.4}, |
| | | | | | | | RHOB^{0.39}, MARCKS^{3.21}, MAX^{2.35}, RAB8A^{2.38}, TRIM37^{0.4}, |
| | | | | | | | MYB^{0.2}, NFYC^{2.16}, NPY1R^{0.33}, GAL^{2.18}, PCNA^{2.51}, LEF1^{3.79}, |
| | | | | | | | SPG21^{2.51}, UFM1^{2.43}, RASD1^{2.07}, POLR2H^{2.05}, RIN2^{2.06}, |
| | | | | | | | ALS2CR2^{2.1}, CAMK2N1^{2.01}, NOLA3^{3.18}, PRKAR1A^{0.28}, |
| | | | | | | | PCID2^{2.44}, TWSG1^{2.3}, RDX^{2.14}, BDNF^{2.36}, RNF25^{0.48}, NSD1^{2.21}, |
| | | | | | | | PHACTR4^{2.16}, SSR3^{2.54}, STAU1^{2.53}, TAF13^{2.15}, C1QBP^{2.72}, |
| | | | | | | | TNFRSF1A^{2.36}, TXN^{3.01}, UBE2I^{2.29}, MALL^{2.51}, CALR^{2.18}, |
| | | | | | | | CAMLG^{0.39}, NCOA3^{0.19}, CSDA^{2.43}, COPS3^{2.45}, CAV1^{2.05}, |
| | | | | | | | KHSRP^{2.49}, RUNX1^{2.17}, AP1S2^{2.93}, MED20^{2.28}, ATP6V1G1^{0.32}, |
| | | | | | | | RBX1^{2.65}, CDC42^{2.76} |
| GO: 0016563 | 0 | 3.59 | 3.07 | 10 | 182 | transcription activator | ETV5^{2.03}, GATA3^{0.28}, CNOT7^{2.29}, MAX^{2.35}, MYB^{0.2}, |
| | | | | | | activity | NFYC^{2.16}, LEF1^{3.79}, COA3^{0.19}, RUNX1^{2.17}, CHURC1^{0.38} |
| GO: 0003924 | 0 | 4.34 | 1.77 | 7 | 105 | GTPase activity | ARL4A^{2.13}, TUBB3^{2.44}, ARF4^{2.25}, RHOB^{0.39}, RND3^{2.29}, |
| | | | | | | | RASD1^{2.07}, CDC42^{2.76} |

Biological Component

| | | | | | | | |
|---|---|---|---|---|---|---|---|
| GO: 0009893 | 0 | 3.21 | 4.53 | 13 | 271 | positive regulation of | EREG^{2.86}, ETV5^{2.03}, GATA3^{0.28}, GJA1^{2.06}, CNOT7^{2.29}, |
| | | | | | | metabolic process | FOXA1^{0.2}, LEF1^{3.79}, NSD1^{2.21}, TNFRSF1A^{2.36}, UBE2D1^{2.19}, |
| | | | | | | | NCOA3^{0.19}, RUNX1^{2.17}, CHURC1^{0.38} |
| GO: 0045935 | 0 | 3.49 | 3.51 | 11 | 210 | positive regulation of | EREG^{2.86}, ETV5^{2.03}, GATA3^{0.28}, CNOT7^{2.29}, FOXA1^{0.2}, |
| | | | | | | nucleobase, nucleoside, | LEF1^{3.79}, NSD1^{2.21}, TNFRSF1A^{2.36}, NCOA3^{0.19}, RUNX1^{2.17}, |
| | | | | | | nucleotide and nucleic | CHURC1^{0.38} |
| | | | | | | acid metabolic process | |
| GO: 0010557 | 0 | 3.3 | 3.7 | 11 | 221 | positive regulation of | EREG^{2.86}, ETV5^{2.03}, GATA3^{0.28}, CNOT7^{2.29}, FOXA1^{0.2}, |
| | | | | | | macromolecule | LEF1^{3.79}, NSD1^{2.21}, TNFRSF1A^{2.36}, NCOA3^{0.19}, |
| | | | | | | biosynthetic process | RUNX1^{2.17}, CHURC1^{0.38} |
| GO: 0007165 | 0 | 1.83 | 28.57 | 43 | 1708 | signal transduction | ARL4A^{2.13}, NAMPT^{2.17}, SPRY1^{2.53}, TUBB3^{2.44}, FRS3^{2.69}, |
| | | | | | | | FOXN3^{2.62}, DCBLD2^{2.54}, CSNK1E^{4.06}, CSNK2B^{2.7}, CNKSR3^{2.48}, |
| | | | | | | | DPYSL2^{2.76}, EREG^{2.86}, GJA1^{2.06}, CNOT7^{2.29}, HMGB2^{2.32}, |
| | | | | | | | IFNGR2^{2.94}, IL8^{2.87}, ITGAE^{2.34}, ARF4^{2.25}, RHOB^{0.39}, RND3^{2.29}, |
| | | | | | | | RAB8A^{2.38}, NPY1R^{0.33}, GOLT1B², GAL^{2.18}, PCNA^{2.51}, LEF1^{3.79}, |
| | | | | | | | SPG21^{2.51}, RASD1^{2.07}, RIN2^{2.06}, PRKAR1A^{0.28}, CXCR7^{0.1}, |
| | | | | | | | TWSG1^{2.3}, RPS6KB1^{0.24}, TNFRSF1A^{2.36}, TXN^{3.01}, UBE2D1^{2.19}, |
| | | | | | | | CAMLG^{0.39}, NCOA3^{0.19}, COPS3^{2.45}, CAV1^{2.05}, MTA1^{2.68}, |
| | | | | | | | CDC42^{2.76} |
| GO: 0048514 | 0 | 5.11 | 1.3 | 6 | 78 | blood vessel | EREG^{2.86}, GJA1^{2.06}, IL8^{2.87}, RHOB^{0.39}, CAV1^{2.05}, RUNX1^{2.17} |
| | | | | | | morphogenesis | |
| GO: 0010628 | 0 | 3.24 | 3.4 | 10 | 203 | positive regulation of | ETV5^{2.03}, GATA3^{0.28}, CNOT7^{2.29}, FOXA1^{0.2}, LEF1^{3.79}, |
| | | | | | | gene expression | NSD1^{2.21}, TNFRSF1A^{2.36}, NCOA3^{0.19}, RUNX1^{2.17}, |
| | | | | | | | CHURC1^{0.38} |
| GO: 0030855 | 0 | 12.91 | 0.28 | 3 | 17 | epithelial cell | GJA1^{2.06}, FOXA1^{0.2}, CAV1^{2.05} |
| | | | | | | differentiation | |
| GO: 0048646 | 0 | 4.66 | 1.42 | 6 | 85 | anatomical structure | EREG^{2.86}, IL8^{2.87}, RHOB^{0.39}, LEF1^{3.79}, TWSG1^{2.3}, RUNX1^{2.17} |
| | | | | | | formation | |
| GO: 0048522 | 0 | 2.18 | 9.72 | 19 | 581 | positive regulation of | NAMPT^{2.17}, EREG^{2.86}, ETV5^{2.03}, GATA3^{0.28}, GJA1^{2.06}, |
| | | | | | | cellular process | CNOT7^{2.29}, FOXA1^{0.2}, GOLT1B², GAL^{2.18}, LEF1^{3.79}, TWSG1^{2.3}, |
| | | | | | | | NSD1^{2.21}, TNFRSF1A^{2.36}, UBE2D1^{2.19}, NCOA3^{0.19}, RUNX1^{2.17}, |
| | | | | | | | PDCD5^{2.49}, CHURC1^{0.38}, CDC42^{2.76} |
| GO: 0042445 | 0 | 7.13 | 0.64 | 4 | 38 | hormone metabolic | GATA3^{0.28}, FOXA1^{0.2}, GAL^{2.18}, SRD5A1^{2.23} |
| | | | | | | process | |
| GO: 0001944 | 0 | 4.38 | 1.51 | 6 | 90 | vasculature | EREG^{2.86}, GJA1^{2.06}, IL8^{2.87}, RHOB^{0.39}, CAV1^{2.05}, RUNX1^{2.17} |
| | | | | | | development | |
| GO: 0048518 | 0 | 6.69 | 0.67 | 4 | 44 | positive regulation of | IL8^{2.87}, RHOB^{0.39}, SPG21^{2.51}, CAV1^{2.05} |
| | | | | | | biological process | |
| GO: 0045893 | 0.01 | 3.21 | 2.71 | 8 | 162 | positive regulation of | GATA3^{0.28}, CNOT7^{2.29}, FOXA1^{0.2}, LEF1^{3.79}, |
| | | | | | | transcription, DNA- | NSD1^{2.21}, TNFRSF1A^{2.36}, NCOA3^{0.19}, RUNX1^{2.17} |
| | | | | | | dependent | |
| GO: 0006357 | 0.01 | 2.54 | 4.7 | 11 | 281 | regulation of | CNOT7^{2.29}, HMGB2^{2.32}, FOXA1^{0.2}, NFYC^{2.16}, LEF1^{3.79}, |
| | | | | | | transcription from RNA | PRKAR1A^{0.28}, NSD1^{2.21}, TNFRSF1A^{2.36}, CSDA^{2.43}, RUNX1^{2.17}, |
| | | | | | | polymerase II promoter | MED20^{2.28} |

Cellular Component

| | | | | | | | |
|---|---|---|---|---|---|---|---|
| GO: 0005794 | 0 | 2.5 | 7.05 | 16 | 413 | Golgi apparatus | TMED2^{2.8}, KDELR2^{2.21}, SYNE1^{2.02}, CHIC2^{3.76}, GJA1^{2.06}, |
| | | | | | | | ARF4^{2.25}, RND3^{2.29}, GOLT1B², GAL^{2.18}, C4orf18^{0.47}, SPG21^{2.51}, |
| | | | | | | | CHPT1^{3.06}, MALL^{2.51}, CAV1^{2.05}, ST3GAL5^{2.18}, AP1S2^{2.93} |
| GO: 0000139 | 0 | 2.84 | 3.81 | 10 | 223 | Golgi membrane | TMED2^{2.8}, GJA1^{2.06}, RND3^{2.29}, GOLT1B², C4orf18^{0.47}, |
| | | | | | | | CHPT1^{3.06}, MALL^{2.51}, CAV1^{2.05}, ST3GAL5^{2.18}, AP1S2^{2.93} |
| GO: 0005798 | 0.01 | 6.22 | 0.72 | 4 | 42 | Golgi-associated vesicle | CHIC2^{3.76}, GJA1^{2.06}, SPG21^{2.51}, AP1S2^{2.93} |
| GO: 0000793 | 0.01 | 5.62 | 0.79 | 4 | 46 | condensed | CENPA^{2.06}, C18orf24^{3.04}, HMGB2^{2.32}, UBE2I^{2.29} |
| | | | | | | chromosome | |

NOTE:
Subscripts denote fold change of (MDA_3A2/MDA_IgG)/(MCF_3A2/MCF_IgG)

Tables A3 and A4, at the end of this document, list genes which are overexpressed in MCF-7 cells, and those which are overexpressed in MDA MB231 cells, respectively. The complete set of genes is also available in the NCBI database (Accession number GSE17820) at the following link: http://www.ncbi.nlm.nih.gov/geo/query/acc.cgi?token=pdsnrqmiawukqlm&acc=GSE17820.

These genes generally fell into three groups. Group 1 consisted of cancer-associated genes which were known HuR targets, such as PTMA mRNA. Group 2 consisted of genes which played a role in cancer but were not known to be HuR targets. Group 3 consisted of genes with an unknown function in cancer, but which may be regulated by HuR. These data revealed that HuR was associated with distinct subsets of mRNAs in ER+ and ER− breast cancer cells.

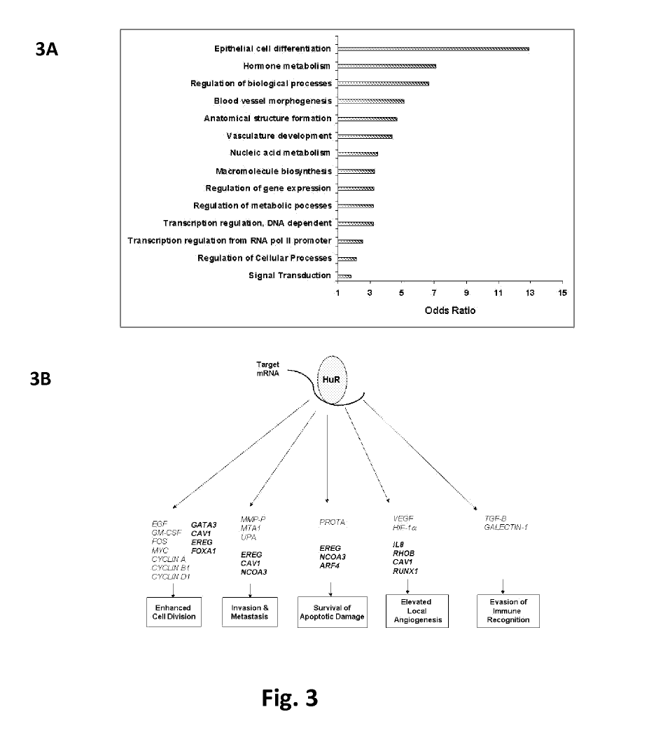

Gene Ontology (GO) analyses of differentially expressed significant genes between ER+ and ER− cells were categorized into Biological Process (BP), Cellular Component (CC), and Molecular Function (MF). GO analyses allows for the identification of gene families that may play significant roles related to these categories in expression profiles. Most of the differentially-expressed genes (155) were found to be more abundant than expected in 14 BP categories (FIG. 3A). Three MF categories consisted of 100 genes with most of these (83) related to protein binding and transcription activator activity. The CC categories contained the least (34) and were primarily associated with the Golgi apparatus. For the complete GO analyses, see Table 1. In Table 1, we list the top HuR-associated mRNAs in the different categories which were approximately 5 fold enriched or greater. As can be seen in FIG. 3B, a partial listing of some of these genes (in bold) are candidate members to be involved in multiple areas of cancer control, as suggested by Hanahan and Weinberg (Cell 2000, 100(1):57-70). We note that though B-ACTIN mRNA was amongst the most abundant of HuR-associated mRNAs in MCF-7 cells, B-ACTIN mRNA levels were only 3.93-fold higher in HuR IP compared to IgG IP, and hence less than the 5-fold cut-off we employed for Table 2. Taken together, novel HuR-controlled genes have been identified, which may play roles in breast carcinogenesis in a cancer subtype-specific fashion.

TABLE 2
HuR Targets Approximately Five-Fold or Greater In Decreasing
Order* Listing of HuR-associated mRNAs in MB-231 and
MCF-7 cell lines.

| | | | | Both Cell |
|---|---|---|---|---|
| MB-231 Cells | MCF-7 Cells | | | Lines |
| CD9 | ACTB | SMNDC1 | MAL2 | CALM2 |
| PTMA | CALM2 | CDK2AP1 | hCG_1781062 | SRRM1 |
| UBE2E2 | JUND | ARL6IP1 | PTMA | CCNI |
| CCNI | ATP6V1G1 | ACTB | HMGB1 | DAZAP2 |
| CKLF | BUB3 | PJA2 | LOC203547 | CD9 |
| SRRM1 | NPM1 | MATR3 | TMC01 | ARL6IP1 |
| STK4 | CXCR7 | ZFP36L1 | SFRS2 | PTMA |
| FKBP1A | TMSL3 | PLOD2 | PPP6C | ATP1B1 |
| PMP22 | EIF4A2 | RPS6KB1 | HSPA1A | MMD |
| CALM2 | TIMEM59 | FOXA1 | PEX11B | TMCO1 |
| MMD | MYB | CD9 | ZNF14 | |
| CSDA | ITGB1 | PARD6B | LOC441087 | |
| CHIC2 | SRRM1 | SNX16 | PUM1 | |
| DAZAP2 | MORF4L1 | TFDP1 | MMD | |
| ZNF22 | GCA | CISD2 | C4orf34 | |
| ATP1B1 | DAZAP2 | G3BP1 | C21orf55 | |
| TRAM1 | NCOA3 | ATP1B1 | SFPQ | |
| ENY2 | PRKAR1A | YBX1 | HIST1H3E | |
| ALKBH5 | CCNI | CSTB | C15orf51 | |
| RAP2A | YWHAZ | PRIM2 | SLC7A1 | |
| TMCO1 | C15orf15 | PCBP2 | ROD1 | |
| ARL6IP1 | SPINT2 | CALMG | YTHDC1 | |

*The complete set of gene are up-loaded to NCBI database at the following link: http://www.ncbi.nlm.nih.gov/geo/query/acc.cgi?token=pdsnrqmiawukqlm&acc=GSE17820. (NCBI Accession number GSE17820).

### Validation of HuR Targets CD9 and CALM2 by Real-Time PCR and Biotin Pull-Down Analyses

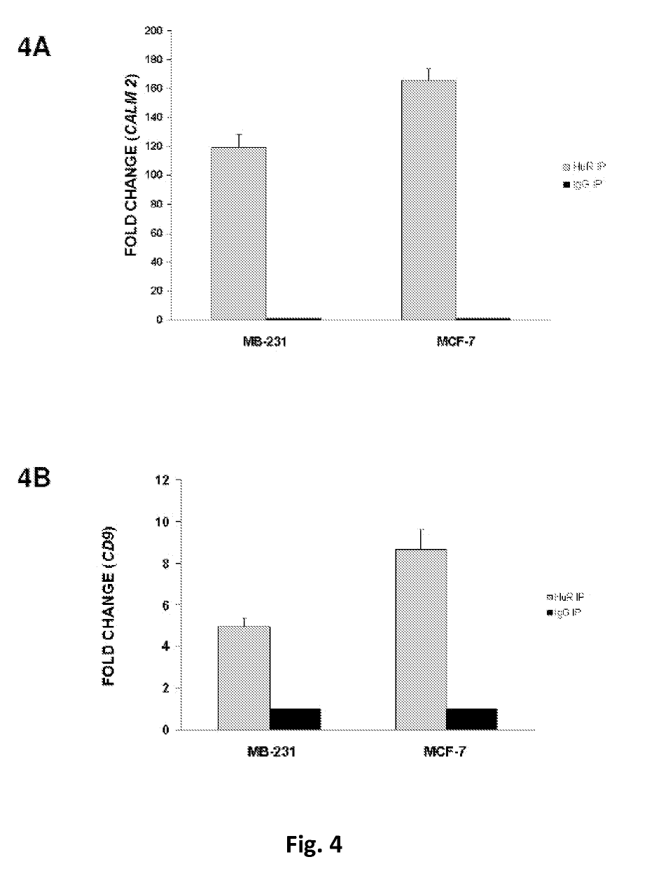

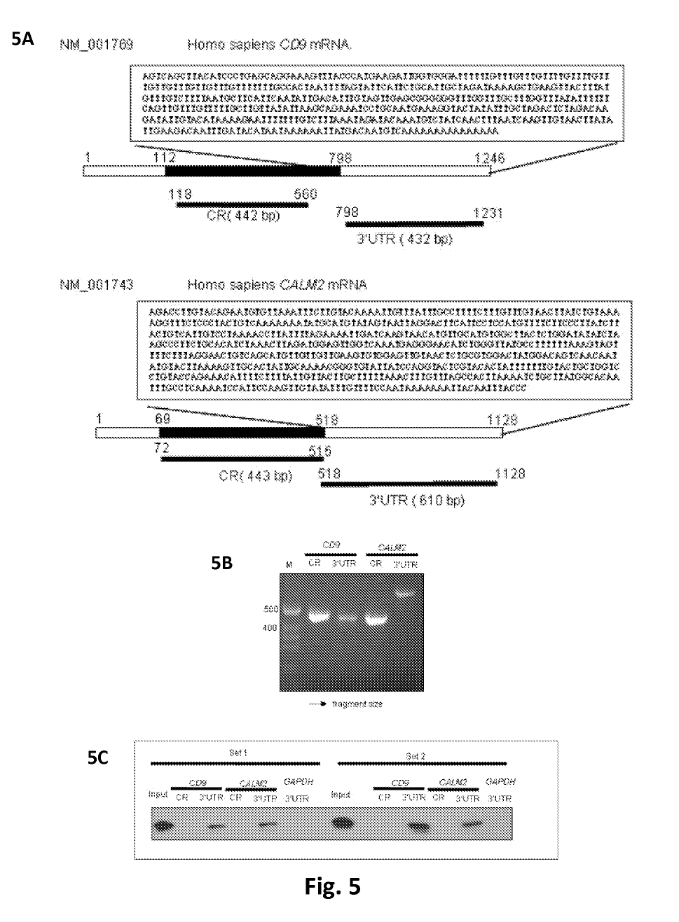

In order to validate HuR binding to genes identified in FIG. 2, we chose two known cancer associated genes, CD9 and CALM2, which were highly expressed in both cell lines. Two independent approaches confirmed the physical interaction between HuR, CD9 and CALM2 mRNAs. Precipitated mRNA from the RIP-Chip experiments were analyzed by RT-PCR. Both CD9 and CALM2 mRNAs were enriched in the HuR RIP by as much as 160-fold (FIGS. 4A and 4B), but not the isotype control IP. We further confirmed HuR binding to CD9 and CALM2 mRNAs by biotin pull-down assays. The relevant portion of the mRNA was transcribed with biotin tags, and incubated with lysates from the two cell lines to probe for interactions with protein. The mixtures were then separated by pull-down assays using streptavidin-coated beads, and HuR levels were analyzed by Western blot analysis. As shown in FIG. 5, HuR specifically interacts with CD9 and CALM2 mRNAs in the 3′UTR regions, but not within the coding region (CR) or with a control biotinylated RNA corresponding to the 3′UTR of the housekeeping control GAPDH mRNA, which is not a target of HuR.

### HuR Differentially Regulates CD9 and CALM2 in MB231 and MCF-7 Cell Lines

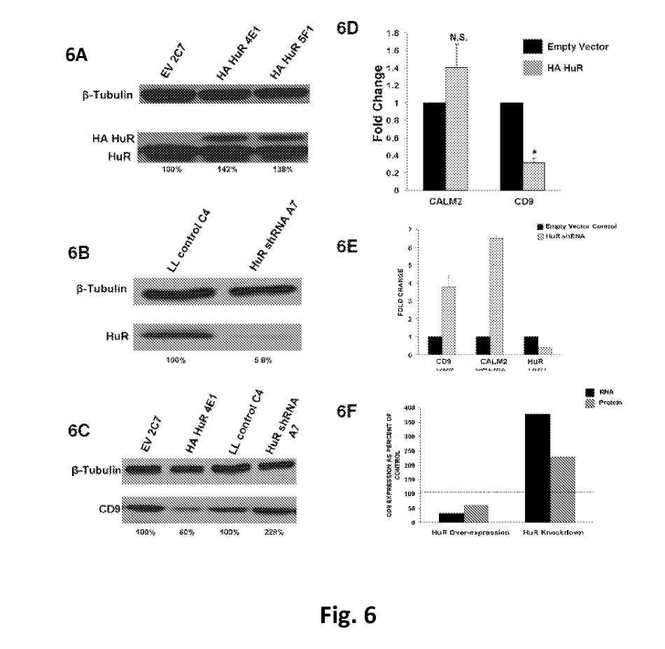

To gain insight into the biological effects of these associations, we studied the consequences of stably increasing or decreasing HuR abundance. Individual MB-231 clones which over- and under-express HuR were established by limiting dilution (FIGS. 6A and 6B). MB-231 cells overexpressed HuR by 140% (FIG. 6A). HuR knock down assays using lentiviral shRNA reported a ˜95% reduction in HuR expression (FIG. 6B). Surprisingly, overexpression of HuR in MB-231 cells caused decreases in both CD9 mRNA and protein levels (FIGS. 6C and 6D). HuR knock down assays, however, reported increases in both CD9 mRNA and protein levels (FIGS. 6C and 6E). This is the opposite of what we predicted, since HuR is generally regarded as a stabilizer of mRNA. In contrast, overexpression of HuR in MB-231 cells did not significantly alter the levels of CALM2 mRNA (FIG. 6D). FIG. 6F shows a graphical analysis, which reveals that HuR over-expression decreases both CD9 mRNA and protein levels, compared to controls (dashed line set at 100%). The HuR shRNA knock-down experiments demonstrate increases in both CD9 mRNA and protein levels above control levels.

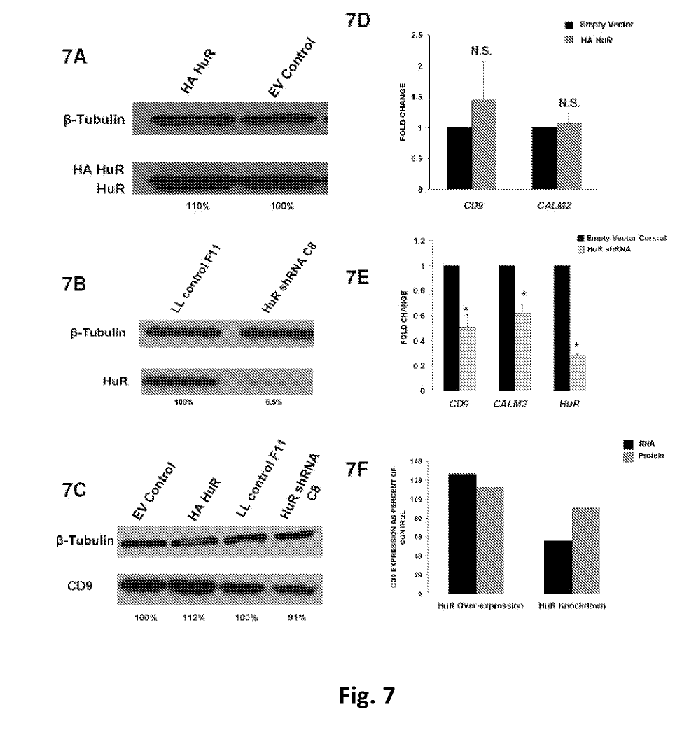

Similar analyses were performed with MCF-7 cells, which demonstrated that the over-expression levels of HA HuR were less than expected, approximately 10%, since this was a pooled population and we were unable to obtain MCF-7 clones which over-express HuR. In contrast, we generated MCF-7 clones with reduced HuR levels (93%) using lentiviral shRNA (FIG. 7B). Western blot analysis of MCF-7 cells which over-express HuR revealed modest increases in CD9 protein levels (FIG. 7C). There are also modest decreases in CD9 protein expression in MCF-7 with reduced HuR levels (FIG. 7C). mRNA levels of CD9 and CALM2 are essentially unchanged in MCF-7 cells which over-express HuR (FIG. 7D). As expected, HuR knock-down in MCF-7 cells using the lentiviral shRNA resulted in significant reductions in both CD9 and CALM2 mRNA levels (FIG. 7E). The right subpanel in FIG. 7E indicates the efficiency of HuR mRNA knock-down, which is consistent with the protein data (FIG. 7B). These results are summarized in FIG. 7F. There are no significant changes seen in CD9 mRNA and CD9 protein for HuR over-expression. There is a more pronounced knock-down, however, in CD9 mRNA in MCF-7 cells which have reduced HuR levels.

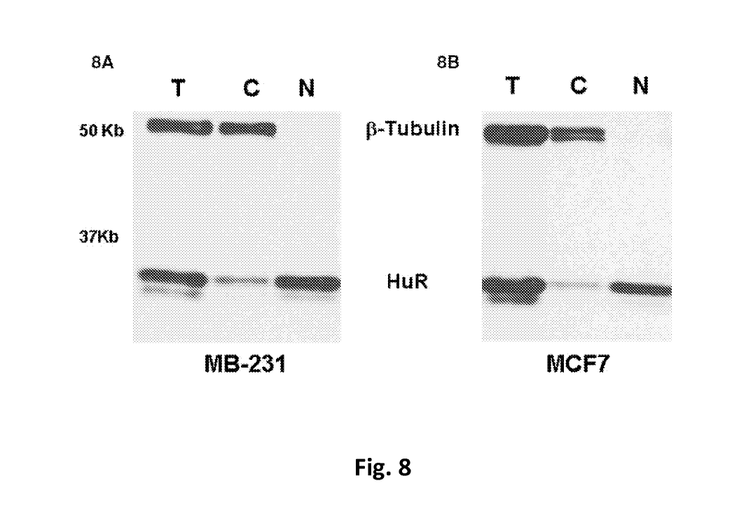

The results of HuR shRNA knock down experiments in MCF-7 cells were as expected, but opposite of those seen for MB-231 cells. Steady-state mRNA levels of CD9 and CALM2 mRNAs decreased, consistent with the hypothesis that HuR generally stabilizes its mRNA targets. One possible explanation of these disparate results is different levels of total cellular or cytoplasmic HuR. We performed nuclear and cytoplasmic fractionation (FIG. 8). These results demonstrate a modest (approximately 10%) greater cytoplasmic levels of HuR in MB-231 cells compared to MCF-7 cells. The total cellular HuR levels are very similar for both MB-231 and MCF-7 cells. Taken together, these results indicated that HuR appeared to differentially regulate the same mRNAs, in a manner dependent upon the cellular milieu.

RIP-Chip technologies were used to define differentially regulated HuR genes in ER+ and ER− breast cancer. Presented is a side-by-side genome-wide comparison of HuR-associated targets in wild-type ER+ and ER− breast cancer cells. These findings demonstrate that HuR interacts with small subsets of genes involved in breast cancer, out of the possible 8% of human genes possessing AREs which are potential targets of HuR. Three broad categories of HuR targets were identified. First, there was a subset of targets only found in ER+ breast cancer. Second, there was a unique subset of HuR targets found only in ER− breast cancer. A third subset consisted of HuR-associated mRNAs common to both forms of breast cancer, many of which were previously described as having roles in cancer.

We selected and validated two HuR targets, CD9 and CALM2 mRNAs, which were found in high abundance in both types of breast cancer. Initially, we employed the previously developed “heat map” signature of HuR binding to gain insight into putative HuR target sequences [Lopez de Silanes I et al., Proc Natl Acad Sci USA 2004, 101(9):2987-2992]. HuR binding was verified by HuR immunoprecipitations, and analyzed by quantitative RT-PCR and biotin pull-down assays. Both targets were enriched in HuR RIPS, compared to isotype control IP reactions. Biotin pull-down assays verified the binding of HuR protein specifically to the 3′UTR regions of both mRNAs, as had been predicted.

CD9, for example, is a tetraspanin molecule which plays important roles in cellular development, activation, growth and motility. It has been implicated in a variety of cancers, including gastric cancers and B cell acute leukemia [Lafleur M A et al., Mol Biol Cell 2009, 20(7):2030-2040; Nakamoto T et al, Gastroenterol 2009, 44(9):889-896; Nishida H et al., Biochem Biophys Res Commun 2009, 382(1):57-62].

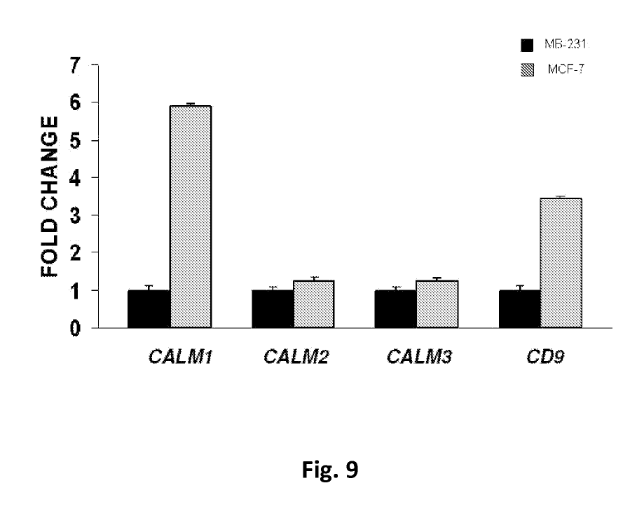

The role of CALM2 in cancer is less well understood, but may be linked to cancer since it is involved in controlling calcium signaling [Coticchia C M et al., Breast Cancer Res Treat 2009, 115(3):545-560; Schmitt J M et al., Mol Cell Biochem 2009, 335(1-2):155-171.1 There are three CALMODULIN genes (CALM1, CALM2 and CALM3) highly expressed in both MB-231 and MCF-7 cell lines (FIG. 9). Although they are encoded by different genes at different chromosomal locations, all three encode the same open reading frame but differ in the 5′ and 3′ untranslated (UTRs) regions [Coticchia CM et al., Breast Cancer Res Treat 2009, 115(3):545-560; Berchtold M W et al., Genomics 1993, 16(2):461-465; Fischer R et al., J Biol Chem 1988, 263(32):17055-17062]. Of the three, only CALM2 mRNA interacts with HuR by RIP analysis. Previously published reports have also indicated the necessity of knocking down all three CALMODULIN mRNAs by siRNA to achieve knock down of the protein [Coticchia C M et al., Breast Cancer Res Treat 2009, 115(3):545-560]. Therefore, differential HuR-associated regulation of the CALMODULIN genes appears to be involved in breast cancer, requiring additional studies at a molecular level.

The regulation of both CD9 and CALM2 target genes appeared to be dependent upon the cellular milieu. To test the functional consequences of HuR binding to these two transcripts, we prepared cells that stably expressed higher or lower amounts of HuR, compared to the parent cells, in both ER+ and ER− breast cancer cell lines. HuR appeared to differentially regulate the expression of CD9 in opposite directions in the two different forms of breast cancer. Specifically, HuR overexpression in ER− breast cancer (MB-231) unexpectedly decreased CD9 mRNA and protein levels, while HuR knock down experiments demonstrated an increase in CD9 mRNA levels. This is usually the opposite of what is predicted for most HuR targets, since HuR is thought to stabilize its mRNA targets and often increases their translation. There did not seem to be similar effects upon CALM2 expression. As expected, knock down of HuR by shRNA decreased expression of CD9 and CALM2 in ER+ breast cancer (MCF-7). Though there are differences in cytoplasmic HuR levels in MB-231 cells as compared with MCF-7, these are modest (10%). This is in keeping, however, with observations that MB-231 cells are more undifferentiated and more aggressive.

Analysis of HuR-associated mRNAs in both ER+ and ER− breast cancer revealed three broad categories of genes. First, there were well known cancer genes, such as PTMA, which are regulated by HuR [Lal A et al., Embo J 2005, 24(10):1852-1862]. Second, there were cancer-related genes, such as CD9 and CALMODULIN, which were not known to be HuR regulated, until this report. Third, there were other genes identified by HuR association with unknown cancer functions, which could represent novel cancer targets. Demonstration of HuR involvement in the regulation of other known cancer genes, such as CD44 and GATA-3, may offer insights into the regulation of these and similar cancer targets Tables A3 and A4. Taken together, these results may reveal insights into post-transcriptional regulation of many genes which are known to be associated with cancer, and facilitate the identification of previously unknown genes with similar or novel roles in regulating genes associated with cancer.

Without being bound by mechanisms of the HuR differential regulation of CD9 and CALM2, it may be involved in microRNA (miRNA) regulation. In a recent report, we described the recruitment by HuR of miRNA let-7 to translationally silence C-MYC expression [Kim H H et al., Genes & Dev 2009, 23: 1743-1748]. It is clear from the findings of laboratories headed by Filipowicz, Steitz and other investigators, that RBPs and miRNAs are involved in intricate associations to affect downstream translational suppression or activation of target mRNAs to help meet cellular needs [Bhattacharyya S N et al., Cell 2006, 125(6):1111-1124; Vasudevan S, Steitz J A, Cell 2007, 128(6):1105-1118]. Sharp and colleagues proposed that different interactions between RBPs and miRNAs may have evolved as a protective mechanism for the cell against environmental stress [Leung A K, Sharp P A, Cell 2007, 130(4):581-585].

A remaining question is why HuR selectively binds to certain genes containing AREs. Our previous work has demonstrated the role that HuR plays in myogenesis by stabilizing the expression of three critical genes involved in myogenesis: MYOD, MYOGENIN, and p21^{cip1} [Figueroa A et al, Mol Cell Biol 2003, 23(14):4991-5004]. HuR overexpression results in precocious muscle differentiation and HuR siRNA knock down prevents muscle differentiation [van der Giessen K et al., J Biol Chem 2003, 278(47):47119-47128]. It is highly probable that there are more than three HuR targets inside these cells. A specific phenotype potentially arises when HuR levels are altered, which may involve interactions with miRNAs.

Our findings share some similarity to earlier reports of HuR RIP-Chip analysis of MCF-7 cells stably transfected with MCT-1 [Mazan-Mamczarz K et al., Oncogene 2008, 27: 6151-6163]. These analyses, however, were not genome-wide and employed transfected cells. Thrombospondin, a well-known anti-angiogenic factor, was identified as a HuR-regulated target. Combined with earlier reports of the role of HuR in regulating, VEGF-α and HIF1α, HuR may be controlling a “posttranscriptional mini-operon” involved in angiogenesis [Levy A P, Trends Cardiovasc Med 1998, 8(6):246-250; Sheflin L G et al., Biochem Biophys Res Commun 2004, 322(2):644-651; Galban S et al., Mol Cell Biol 2008, 28(1):93-107]. Xenograft animal models can also be used to investigate the role of HuR in breast cancer angiogenesis. The role of HuR in influencing expression of various biomarkers can also be evaluated in breast tumors in vivo.

Post-transcriptional gene regulation is increasingly being appreciated as a driver of malignant transformation. The roles of both RBPs and miRNAs (so-called oncomirs) are being recognized in cancer [Esquela-Kerscher A, Slack F J, Nat Rev Cancer 2006, 6(4):259-269]. Many reports have described alterations in miRNA expression profile and function as contributing to breast cancer malignant transformation and metastasis [Iorio M V et al., Cancer Res 2005, 65(16):7065-7070; Ma L et al., Nature 2007, 449(7163):682-688; Ma L, Weinberg R A, Trends Genet 2008, 24(9):448-456; Tavazoie S F et al., Nature 2008, 451(7175):147-152]. HuR RIP-Chip analysis may shed further light into malignant breast cancer transformation by identifying HuR associated mRNAs.

The HuR-associated biomarkers may also be used in applications for identifying drug resistance, specifically, tamoxifen resistance. Keene and colleagues have described a potential mechanistic link between HuR expression and tamoxifen drug resistance [Hostetter C et al., Cancer Biol Ther 2008, 7(9)]. As breast cancer cells acquire tamoxifen resistance, there are increased levels of cytoplasmic HuR expression. Increased cytoplasmic HuR levels have previously been described in situations where HuR actively influences expression of cytoplasmic targets [Atasoy U et al., J Cell Sci 1998, 111 (Pt 21):3145-3156; Atasoy U et al., J Immunol 2003, 171(8):4369-4378; Casolaro V et al., The Journal of Allergy and Clinical Immunology 2008, 121(4):853-859 e854]. Drug resistance could be reversed by using siRNA to knock down HuR expression, whereas exogenous overexpression of HuR could cause cells to become resistant to tamoxifen. HuR may be coordinately regulating genes which may allow a cell to acquire tamoxifen resistance. HuR-associated target genes in ER+ cells is of particular interest.

### Conclusion

In summary, using RIP-Chip analysis, we have performed a genome-wide comparison of HuR-associated targets in wild type ER+ and ER− breast cancer for the first time. We have identified novel HuR targets and have gained insight into HuR's potential role in regulating known cancer genes. We found distinct, differentially expressed subsets of HuR cancer related genes in ER+ and ER− breast cancer cell lines. Based on our observations, the enhanced expression of these mRNA subsets by HuR can influence many of the acquired capabilities of cancer cells. HuR's role in regulating these genes may provide novel methods to facilitate the diagnosis of breast cancer and enhance the ability of physicians to monitor the progress of therapies designed to treat breast cancer in patients.

TABLE A3
Genes over expressed in MCF-7cells (Top Genes of Interest, Fold = MCF.3A2/MCF.IgG)

| | | | | | | | | PROB OF | FOLD |
|---|---|---|---|---|---|---|---|---|---|
| LOCUS LINK ID | ID | GENESYMBOL | AVEEXPR | T | P. VALUE | ADJ. P. VAL | B | DIFF EXP | CHANGE |
| 60 | ZuropJSp8XsR4fiFL4 | ACTB | 10.04 | 5.11 | 0 | 0.01 | 0.32 | 0.58 | 12.94 |
| 805 | Kvvgu6L7B3m6HOhLQQ | CALM2 | 9.75 | 8.23 | 0 | 0 | 4.61 | 0.99 | 11.99 |
| 3727 | 3nGLUT17_w1_vZWv94 | JUND | 10.42 | 7.68 | 0 | 0 | 3.95 | 0.98 | 11.31 |
| 9550 | uF7uCSSUl8Cy1PfnDo | ATP6V1G1 | 8.68 | 17.28 | 0 | 0 | 11.64 | 1 | 11.06 |
| 9184 | lSy3hs.Vfe1XLCVL54 | BUB3 | 9.35 | 7.35 | 0 | 0 | 3.53 | 0.97 | 10.23 |
| 4869 | 3vtSvc_UIO77UA5e.I | NPM1 | 9.64 | 7.11 | 0 | 0 | 3.22 | 0.96 | 10.15 |
| 57007 | E3u67.sWkajOgYAef4 | CXCR7 | 8.1 | 47.13 | 0 | 0 | 17.97 | 1 | 9.96 |
| 7117 | upUK7Xkp7Dkvw0i5T8 | TMSL3 | 10.1 | 8.46 | 0 | 0 | 4.87 | 0.99 | 9.77 |
| 1974 | KoV75wlUkJDXKyr8NU | EIF4A2 | 9.17 | 7.05 | 0 | 0 | 3.14 | 0.96 | 9.71 |
| 9528 | BieNPnX3RdeU4x7S8U | TMEM59 | 8.84 | 11.98 | 0 | 0 | 8.24 | 1 | 9.55 |
| 4602 | Ku.kHqiEgHuvjJS0eU | MYB | 8.41 | 26.22 | 0 | 0 | 14.93 | 1 | 9.47 |
| 3688 | WunOQSd0XGYt8f4vLk | ITGB1 | 8.91 | 5.76 | 0 | 0.01 | 1.33 | 0.79 | 9.25 |
| 10250 | 3Qf0iXfs.oKegqVIf4 | SRRM1 | 8.89 | 8.84 | 0 | 0 | 5.29 | 0.99 | 9.12 |
| 10933 | 355S7.Q46EEioznsi4 | MORF4L1 | 8.99 | 9.38 | 0 | 0 | 5.87 | 1 | 9 |
| 25801 | xvrrv4q_nIDgJej.uU | GCA | 8.48 | 24.81 | 0 | 0 | 14.54 | 1 | 8.73 |
| 9802 | 0Lt45pR09p1Ug9ch6s | DAZAP2 | 9.11 | 8.19 | 0 | 0 | 4.56 | 0.99 | 8.69 |
| 8202 | 9UTgOHzqo64nuHn_eE | NCOA3 | 8.34 | 19.45 | 0 | 0 | 12.65 | 1 | 8.38 |
| 5573 | cuQcHh3vPjV915X9Uo | PRKAR1A | 8.62 | 13.69 | 0 | 0 | 9.52 | 1 | 8.36 |
| 10983 | oioTn1X7UX_SXv3tOw | CCNI | 9.9 | 7.57 | 0 | 0 | 3.81 | 0.98 | 8.2 |
| 7534 | rpFefX_fk1RIc.V01w | YWHAZ | 9.06 | 6.11 | 0 | 0.01 | 1.84 | 0.86 | 8.19 |
| 51187 | frL7o56o4geDDf5ei4 | C15orf15 | 8.9 | 10.38 | 0 | 0 | 6.85 | 1 | 7.99 |
| 10653 | xp59et6So6v5.oDXco | SPINT2 | 8.63 | 8.29 | 0 | 0 | 4.68 | 0.99 | 7.81 |
| 10285 | 3Svt5P767C4E00S814 | SMNDC1 | 8.71 | 13.04 | 0 | 0 | 9.05 | 1 | 7.81 |
| | Qi_4HrqWzsEnhQbgjE | | 10 | 6.56 | 0 | 0 | 2.49 | 0.92 | 7.7 |
| 8099 | rSCAiQVFAXBChVYEf0 | CDK2AP1 | 9.55 | 5.74 | 0 | 0.01 | 1.3 | 0.79 | 7.6 |
| 23204 | ZKnvriJIfiuOMvpd60 | ARL6IP1 | 8.7 | 8.82 | 0 | 0 | 5.27 | 0.99 | 7.48 |
| 60 | 6EoLV_U1wCUVR93cKI | ACTB | 9.62 | 4.8 | 0 | 0.02 | −0.17 | 0.46 | 7.4 |
| | udBJ1LwOf4zLp1.kiU | | 8.23 | 16.17 | 0 | 0 | 11.05 | 1 | 7.17 |
| 9867 | iJUrcDsOvr8_9zBVJU | PJA2 | 8.44 | 7.3 | 0 | 0 | 3.46 | 0.97 | 7.14 |
| 9782 | HpTDXI5GfcPTsXkTuE | MATR3 | 8.73 | 7.39 | 0 | 0 | 3.58 | 0.97 | 7.13 |
| 677 | l6PUrei.1DvDsBIHpE | ZFP36L1 | 8.64 | 9.3 | 0 | 0 | 5.78 | 1 | 6.82 |
| 5352 | uEC_Jfn31v_V.t2dc | PLOD2 | 8.51 | 9.3 | 0 | 0 | 5.78 | 1 | 6.74 |
| 6198 | QPfSeXzyi5zirBF73k | RPS6KB1 | 8.36 | 15.34 | 0 | 0 | 10.57 | 1 | 6.68 |
| 3169 | ZlNu5_.TUu85H9RL6E | FOXA1 | 8.29 | 22.91 | 0 | 0 | 13.96 | 1 | 6.58 |
| 928 | rWSgWYjrci0nxNXiSg | CD9 | 8.79 | 10.25 | 0 | 0 | 6.72 | 1 | 6.53 |
| | fgq.Uoebt514ne.ws4 | | 8.54 | 6.36 | 0 | 0 | 2.2 | 0.9 | 6.38 |
| 84612 | WN11QLno_Sk4mXJSgk | PARD6B | 8.81 | 4.95 | 0 | 0.02 | 0.06 | 0.51 | 6.15 |
| 64089 | cy75e3vcCYFJR.9Dek | SNX16 | 8.36 | 17.56 | 0 | 0 | 11.78 | 1 | 6.11 |
| | 3_dx6HGuKOu4VTM4.0 | | 9.69 | 4.96 | 0 | 0.02 | 0.08 | 0.52 | 6.11 |
| 7027 | TFXnpoyQh3ui.vS6xo | TFDP1 | 9.31 | 4.47 | 0 | 0.03 | −0.73 | 0.32 | 6.1 |
| 493856 | 3GcqHr1KlETMUA3lTE | CISD2 | 8.94 | 9.47 | 0 | 0 | 5.96 | 1 | 6.09 |
| 10146 | l693.PjqTkurvH6A6U | G3BP1 | 8.89 | 5.69 | 0 | 0.01 | 1.22 | 0.77 | 6.05 |
| | 0XXK56jxK7iUe70lDE | | 9.62 | 4.13 | 0 | 0.05 | −1.31 | 0.21 | 6.04 |
| 481 | unu3iN6N5U0f6cuEqc | ATP1B1 | 9 | 5.7 | 0 | 0.01 | 1.24 | 0.78 | 5.95 |
| 4904 | QrnhBSrkogQrIUuKSA | YBX1 | 8.91 | 5.64 | 0 | 0.01 | 1.14 | 0.76 | 5.73 |
| 1476 | 3e78KW7T0IlK62aoQE | CSTB | 8.8 | 5.38 | 0 | 0.01 | 0.75 | 0.68 | 5.69 |
| 5558 | lbVIueo8a_4lHuXpf8 | PRIM2 | 9.99 | 3.75 | 0 | 0.08 | −1.97 | 0.12 | 5.69 |
| 5094 | c12iGrpOqJJyBDkj00 | PCBP2 | 8.67 | 9.28 | 0 | 0 | 5.76 | 1 | 5.68 |
| 819 | ZrdJSVyIeffu.u097U | CAMLG | 8.5 | 10.8 | 0 | 0 | 7.24 | 1 | 5.58 |
| 114569 | fengk1X6LlOzC_pzyI | MAL2 | 8.62 | 11.46 | 0 | 0 | 7.81 | 1 | 5.52 |
| 653226 | B4RV5U.3t.DwUK7yu8 | hCG_1781062 | 8.56 | 6.73 | 0 | 0 | 2.72 | 0.94 | 5.43 |
| 5757 | QQ3z1iT1LB..uzsfJ4 | PTMA | 9.07 | 6.48 | 0 | 0 | 2.37 | 0.91 | 5.41 |
| 3146 | x5P787D9KKDHgTeLXo | HMGB1 | 8.56 | 5.65 | 0 | 0.01 | 1.16 | 0.76 | 5.34 |
| 203547 | Ty5Xhyqij_jueT9CW4 | LOC203547 | 8.99 | 5.55 | 0 | 0.01 | 1 | 0.73 | 5.2 |
| 54499 | uYd0KR7s5XkL6e3OJM | TMCO1 | 8.53 | 9.17 | 0 | 0 | 5.65 | 1 | 5.12 |
| 6427 | 9Vj517sCOX7bkgEDp4 | SFRS2 | 8.91 | 6.2 | 0 | 0 | 1.97 | 0.88 | 5.05 |
| | 3bLQpTO7qY6IcsqcpU | | 9.13 | 5 | 0 | 0.02 | 0.14 | 0.53 | 4.93 |
| | oVIueo8S_4lHuXrf38 | | 9.45 | 3.75 | 0 | 0.08 | −1.97 | 0.12 | 4.88 |
| 5537 | T0upGOh1A5dC87MXtU | PPP6C | 8.7 | 9.12 | 0 | 0 | 5.6 | 1 | 4.84 |
| 3303 | oon0If5P1yz97_0vdA | HSPA1A | 7.81 | 11.48 | 0 | 0 | 7.83 | 1 | 4.83 |
| 8799 | fXfXV87cXRQXZ00.pU | PEX11B | 8.28 | 11.42 | 0 | 0 | 7.78 | 1 | 4.83 |
| 7561 | lKUJ_nTlzLVJH_opQ | ZNF14 | 9.65 | 3.78 | 0 | 0.08 | −1.91 | 0.13 | 4.75 |
| 441087 | 3_v4Ax_iKWruunRl7o | LOC441087 | 9.64 | 3.92 | 0 | 0.06 | −1.67 | 0.16 | 4.69 |
| 9698 | rtyX5WJ.XxDSJV3Rfs | PUM1 | 8.36 | 10.48 | 0 | 0 | 6.94 | 1 | 4.67 |
| 23531 | 6p_X8jaueM_Xv1yw6k | MMD | 8.74 | 8.14 | 0 | 0 | 4.49 | 0.99 | 4.64 |
| 201895 | EujpL.ey.6oe6yd_j4 | C4orf34 | 8.13 | 14.77 | 0 | 0 | 10.22 | 1 | 4.63 |
| 54943 | 3..iEi3R1JerhIkIdY | C21orf55 | 9.53 | 5.76 | 0 | 0.01 | 1.32 | 0.79 | 4.61 |
| 6421 | BI_6Dq7CEPrKq4C6v4 | SFPQ | 8.42 | 6.19 | 0 | 0 | 1.97 | 0.88 | 4.56 |
| | rOj_KbuCgz916dxzQw | | 8.34 | 10.12 | 0 | 0 | 6.6 | 1 | 4.56 |
| | 95Lo1SR.qKUKaujTcI | | 9.92 | 4.66 | 0 | 0.02 | −0.41 | 0.4 | 4.55 |
| 8353 | Qeg9LG4ofofrqRIOTc | HIST1H3E | 8.03 | 13.27 | 0 | 0 | 9.22 | 1 | 4.52 |
| 196968 | EJ0RR5LUl6uEiYB1N0 | C15orf51 | 9.93 | 4.83 | 0 | 0.02 | −0.14 | 0.47 | 4.51 |
| 6541 | EdV._eEEe7E_FH1xTE | SLC7A1 | 8.53 | 4.41 | 0 | 0.03 | −0.82 | 0.31 | 4.5 |
| 9991 | uJEnKJd4T7eu.xut70 | ROD1 | 8.62 | 4.82 | 0 | 0.02 | −0.15 | 0.46 | 4.5 |
| 91746 | TPXO9LJuvjnPvyX1XU | YTHDC1 | 8.28 | 11.2 | 0 | 0 | 7.59 | 1 | 4.48 |
| 1979 | BslHrteoP3r6P65Xgc | EIF4EBP2 | 8.6 | 10.02 | 0 | 0 | 6.51 | 1 | 4.43 |
| | QJdcrnqPEruJZ7vSUM | | 9.27 | 3.56 | 0 | 0.1 | −2.3 | 0.09 | 4.42 |
| 55954 | W1_p0JzXVF0l3QMnqE | ZMAT5 | 8.87 | 3.57 | 0 | 0.1 | −2.28 | 0.09 | 4.39 |
| 63905 | o1SeiQ915dJfeLJ6hw | MANBAL | 9.83 | 3.89 | 0 | 0.07 | −1.72 | 0.15 | 4.36 |
| 10776 | ldJER5S31UM3t13Q9U | ARPP-19 | 8.66 | 5.89 | 0 | 0.01 | 1.52 | 0.82 | 4.33 |
| 523 | 9jjkvez8_57t61wuiU | ATP6V1A | 8.08 | 7.98 | 0 | 0 | 4.31 | 0.99 | 4.32 |
| 644316 | uIjqv_dLQUlSnouS64 | FLJ43315 | 9.62 | 4.53 | 0 | 0.03 | −0.63 | 0.35 | 4.3 |
| 5870 | x1XT6BAF8B6iBfLVd0 | RAB6A | 8.49 | 4.03 | 0 | 0.06 | −1.48 | 0.18 | 4.28 |
| 6637 | 6p70kiAVO13dTKl7.E | SNRPG | 8.8 | 4 | 0 | 0.06 | −1.54 | 0.18 | 4.22 |
| 92014 | lXJ52op4pKIN398PpQ | MCART1 | 8.97 | 4.14 | 0 | 0.05 | −1.29 | 0.22 | 4.22 |
| 4591 | KlQBL5QH_U_515P7v4 | TRIM37 | 7.83 | 17.14 | 0 | 0 | 11.57 | 1 | 4.2 |
| | NXbzUdlml1QLnqPMjs | | 9.96 | 3.91 | 0 | 0.07 | −1.69 | 0.16 | 4.19 |
| 64081 | QovYhSXqQRJiB_3c8A | PBLD | 9.01 | 4.03 | 0 | 0.06 | −1.47 | 0.19 | 4.18 |
| | KOh3bXtFSnouSaZDdo | | 9.79 | 4.1 | 0 | 0.05 | −1.35 | 0.21 | 4.16 |
| 6612 | KTL3lz7X1eKdT55_uk | SUMO3 | 8.85 | 3.67 | 0 | 0.09 | −2.1 | 0.11 | 4.13 |
| | TC6K_S4jZhHkdyXqSw | | 9.21 | 3.63 | 0 | 0.09 | −2.18 | 0.1 | 4.12 |
| 56951 | xJ6CCltTXt36mhLsf0 | C5orf15 | 8.35 | 9.64 | 0 | 0 | 6.13 | 1 | 4.1 |
| | Kr6LkumY3SeHklXn1Q | | 9.16 | 3.75 | 0 | 0.08 | −1.96 | 0.12 | 4.1 |
| | Tnzt7MoO0S5COeclSk | | 8.61 | 5.23 | 0 | 0.01 | 0.51 | 0.62 | 4.09 |
| 11254 | WkSP0Ei5_kz7KudDro | SLC6A14 | 7.8 | 10.79 | 0 | 0 | 7.23 | 1 | 4.07 |
| 161291 | 0knRyVFXXc.6sIg.HE | TMEM30B | 7.67 | 12.77 | 0 | 0 | 8.85 | 1 | 4.05 |
| | 0ug6VOXstUnHainSSQ | | 9.47 | 3.96 | 0 | 0.06 | −1.6 | 0.17 | 4.04 |
| 6431 | Wlx.h.xvVPXu8UX11Y | SFRS6 | 8.76 | 4.25 | 0 | 0.04 | −1.09 | 0.25 | 4.04 |
| | cm4lFmVpfyDn8P93SI | | 8.83 | 4.14 | 0 | 0.05 | −1.29 | 0.22 | 4.03 |
| 388 | TkrTkRL.jAqSwQs_qU | RHOB | 8.24 | 14.28 | 0 | 0 | 9.91 | 1 | 4.01 |
| 3423 | KO51fUnriKKLyJ62.4 | IDS | 9.77 | 5.98 | 0 | 0.01 | 1.66 | 0.84 | 4 |
| 6009 | WpIADoapJ9EKQt9Odo | RHEB | 8.76 | 6.36 | 0 | 0 | 2.2 | 0.9 | 4 |
| | 3fz69f76kQGfSfhqtU | | 7.92 | 11.79 | 0 | 0 | 8.09 | 1 | 3.98 |
| 22856 | EEbT6Knmz_wMl450.o | CHSY1 | 8.33 | 4.95 | 0 | 0.02 | 0.06 | 0.52 | 3.98 |
| 55322 | 3eF5TAxUCMwHt9RZVU | C5orf22 | 8.79 | 4.79 | 0 | 0.02 | −0.2 | 0.45 | 3.98 |
| 9349 | 61JLrT2EGAtMWyAI6Y | RPL23 | 8.86 | 4.09 | 0 | 0.05 | −1.38 | 0.2 | 3.95 |
| | cqP908kSr1CVAW4I6I | | 8 | 11.93 | 0 | 0 | 8.2 | 1 | 3.95 |
| 83990 | NqPEruJRLl6VPfi.4w | BRIP1 | 8.79 | 3.79 | 0 | 0.08 | −1.9 | 0.13 | 3.94 |
| 7358 | EVK3TX0oP_S9DiCKiE | UGDH | 8.1 | 8.26 | 0 | 0 | 4.63 | 0.99 | 3.93 |
| | upama6dEf0ztde_8wk | | 8.65 | 4.22 | 0 | 0.04 | −1.15 | 0.24 | 3.92 |
| 11177 | cX4LnsUuenkrPC1C.M | BAZ1A | 7.95 | 8.35 | 0 | 0 | 4.74 | 0.99 | 3.86 |
| 645895 | 07pmZey1Scf6KeKQig | LOC645895 | 10.25 | 3.65 | 0 | 0.09 | −2.15 | 0.1 | 3.85 |
| | xeo8Sk4FFmXpVb3Xx4 | | 8.83 | 3.73 | 0 | 0.08 | −1.99 | 0.12 | 3.84 |
| 6120 | c_d7RUp4LkukS0qVPk | RPE | 9.63 | 4.22 | 0 | 0.04 | −1.15 | 0.24 | 3.8 |
| 1129 | cjfMdel0LjXXl0t1AI | CHRM2 | 9.11 | 4.13 | 0 | 0.05 | −1.31 | 0.21 | 3.79 |
| 1051 | 3h4ZBUbsHtJaleLDZ8 | CEBPB | 8.43 | 8.96 | 0 | 0 | 5.42 | 1 | 3.78 |
| 81671 | Tp55MecDpF3qPpcXqg | TMEM49 | 8.22 | 13.43 | 0 | 0 | 9.33 | 1 | 3.75 |
| 3606 | 07riWI_RT5ncl6kEEk | IL18 | 8.99 | 5.43 | 0 | 0.01 | 0.82 | 0.69 | 3.74 |
| | xoleNeUeR1JWphIuIU | | 9.16 | 3.84 | 0 | 0.07 | −1.81 | 0.14 | 3.71 |
| 56943 | rh_ungNHUApIlesXhI | ENY2 | 8.7 | 6.15 | 0 | 0.01 | 1.9 | 0.87 | 3.68 |
| 374900 | HAuNld.pdUC56r8Sn4 | ZNF568 | 9.12 | 3.62 | 0 | 0.09 | −2.19 | 0.1 | 3.66 |
| 6789 | B4rSS.s4hMn11PlVHU | STK4 | 8.41 | 6.58 | 0 | 0 | 2.51 | 0.93 | 3.64 |
| 23471 | rvRN_9VP3R_7RSe.uU | TRAM1 | 8.54 | 5.47 | 0 | 0.01 | 0.88 | 0.71 | 3.64 |
| | NCtOUeR1JenhLiHe3Q | | 8.99 | 3.63 | 0 | 0.09 | −2.18 | 0.1 | 3.63 |
| 2625 | rkl7nirJLs4nFJuTtI | GATA3 | 7.68 | 13.06 | 0 | 0 | 9.07 | 1 | 3.62 |
| 6202 | oA0kt16KJL1JKkJ9_Y | RPS8 | 9.06 | 12.8 | 0 | 0 | 8.87 | 1 | 3.62 |
| | 3jtH4VT87sokcRT6.U | | 8.54 | 5.49 | 0 | 0.01 | 0.92 | 0.71 | 3.61 |
| 7570 | fV_F33pPde53hAeJSU | ZNF22 | 8.95 | 4.36 | 0 | 0.04 | −0.91 | 0.29 | 3.58 |
| 8766 | x_3fmudOO7qkRoKT54 | RAB11A | 8.16 | 7.97 | 0 | 0 | 4.3 | 0.99 | 3.56 |
| 2764 | TlKkvVHj8jrUIw3T0o | GMFB | 8.61 | 4.74 | 0 | 0.02 | −0.27 | 0.43 | 3.55 |
| 618 | rooyfiVKL2IXl6kMyY | BCYRN1 | 8.83 | 4 | 0 | 0.06 | −1.53 | 0.18 | 3.54 |
| 25862 | N1ycfqKeK6iD50JUos | USP49 | 8.4 | 4.34 | 0 | 0.04 | −0.95 | 0.28 | 3.52 |
| 8763 | TvI.5C3EDid_QSB0SU | CD164 | 8.26 | 11.12 | 0 | 0 | 7.52 | 1 | 3.51 |
| 8323 | KSxf3SE.7IPT9pJ0co | FZD6 | 8.1 | 7.38 | 0 | 0 | 3.57 | 0.97 | 3.5 |
| 10092 | 0YsncAQFF4UIqC4n7k | ARPC5 | 8.31 | 7.17 | 0 | 0 | 3.31 | 0.96 | 3.47 |
| 221786 | fuzJSu66fGXsNUkuog | C7orf38 | 9.43 | 4.39 | 0 | 0.04 | −0.87 | 0.3 | 3.46 |
| 25978 | 3oojO7BevD_o66E.6k | CHMP2B | 8.26 | 6.28 | 0 | 0 | 2.08 | 0.89 | 3.44 |
| 6882 | ugdep8LhXVF0t14snI | TAF11 | 8.71 | 3.55 | 0 | 0.1 | −2.31 | 0.09 | 3.43 |
| 9584 | EbiLespAkt4gIDKA5I | RBM39 | 8.51 | 4.24 | 0 | 0.04 | −1.12 | 0.25 | 3.43 |
| 4886 | Zog_qRUSg4IiAknVwc | NPY1R | 7.84 | 18.81 | 0 | 0 | 12.37 | 1 | 3.43 |
| | lz3dA9fim4lFmVJe10 | | 8.72 | 3.59 | 0 | 0.1 | −2.25 | 0.1 | 3.42 |
| 57092 | uopJ9Ie.z66yefnit8 | PCNP | 8.44 | 5.57 | 0 | 0.01 | 1.04 | 0.74 | 3.42 |
| | BUeCUsqeDFU7KBIdJE | | 7.74 | 22.2 | 0 | 0 | 13.71 | 1 | 3.4 |
| 139886 | Kz54_x6_fvh7HPSOk | SPIN4 | 8.15 | 8.13 | 0 | 0 | 4.48 | 0.99 | 3.4 |
| 8161 | ESkXp4u56LijfgSAfU | COIL | 8.12 | 5.9 | 0 | 0.01 | 1.53 | 0.82 | 3.37 |
| 55153 | 3TT_J5fTHr9dJfX0l4 | SDAD1 | 8.32 | 5.33 | 0 | 0.01 | 0.68 | 0.66 | 3.36 |
| 6613 | Kf.7Gye8TsqQ3t.Cyo | SUMO2 | 9.7 | 7.45 | 0 | 0 | 3.66 | 0.97 | 3.35 |
| 114908 | oqeOni_zvHB_leHr7k | TMEM123 | 9 | 3.61 | 0 | 0.09 | −2.22 | 0.1 | 3.33 |
| 250 | T3OZey1ScVKKeCSjDY | ALPP | 9.07 | 4.02 | 0 | 0.06 | −1.5 | 0.18 | 3.33 |
| 10890 | BKn_Vf97C3fqNe7IJ4 | RAB10 | 8.11 | 7.56 | 0 | 0 | 3.8 | 0.98 | 3.33 |
| | rm2n1SLnqPErtJR5nQ | | 8.89 | 3.79 | 0 | 0.08 | −1.9 | 0.13 | 3.31 |
| 7019 | HlBzpe6NA1JeeXn.zo | TFAM | 8.3 | 7.02 | 0 | 0 | 3.11 | 0.96 | 3.28 |
| 286148 | N5KS7F0r4d7E3gy4tE | DPY19L4 | 8.19 | 6.92 | 0 | 0 | 2.97 | 0.95 | 3.27 |
| 9167 | fSofivk._JOq5KJf3o | COX7A2L | 8.12 | 12.25 | 0 | 0 | 8.45 | 1 | 3.24 |
| 55319 | EIrK.z_6IiL4I8qBS4 | FLJ11184 | 8.05 | 6.6 | 0 | 0 | 2.54 | 0.93 | 3.22 |
| 54407 | BvIpQQ9yzp_kCLnEU | SLC38A2 | 8.4 | 6.37 | 0 | 0 | 2.22 | 0.9 | 3.21 |
| 10276 | ZqbssL7IKdiqG6Hz_U | NET1 | 8.25 | 4.9 | 0 | 0.02 | −0.02 | 0.5 | 3.16 |
| | Wt097RUr6LkuoIn69E | | 9.01 | 3.64 | 0 | 0.09 | −2.16 | 0.1 | 3.16 |
| 7325 | EiHe.NHJfe1dWSCHvo | UBE2E2 | 8.51 | 4.73 | 0 | 0.02 | −0.29 | 0.43 | 3.14 |
| 6138 | QrxUd7UdUynEgAtEJk | RPL15 | 8.18 | 5.64 | 0 | 0.01 | 1.14 | 0.76 | 3.13 |
| | o57uHOPXCP1KnwqcH0 | | 8.06 | 9.44 | 0 | 0 | 5.93 | 1 | 3.12 |
| 4893 | Hr.Uil7.qn9UogI4B4 | NRAS | 8.34 | 4.51 | 0 | 0.03 | −0.66 | 0.34 | 3.12 |
| 55142 | QLTjRlHmcSXqYRLCFc | CEP27 | 8.28 | 3.66 | 0 | 0.09 | −2.12 | 0.11 | 3.11 |
| 114882 | NXkuwsSRHtA9.18K5E | OSBPL8 | 7.85 | 7.98 | 0 | 0 | 4.3 | 0.99 | 3.07 |
| 136051 | Z6ijZeJUqK0KeS4kOM | ZNF786 | 8.41 | 3.73 | 0 | 0.08 | −2 | 0.12 | 3 |
| 79693 | 0C3Sunm7rp05ew1QT0 | YRDC | 9.1 | 5.07 | 0 | 0.02 | 0.26 | 0.57 | 2.99 |
| 80790 | 339VXWtUx9Ekd65qCA | CMIP | 8.57 | 4.06 | 0 | 0.05 | −1.42 | 0.19 | 2.96 |
| 3646 | uJK0inXB6kHQj3p9x4 | EIF3E | 8.59 | 3.88 | 0 | 0.07 | −1.73 | 0.15 | 2.95 |
| 29887 | lsC9OU1KT8ImNdNX0k | SNX10 | 7.85 | 6.55 | 0 | 0 | 2.47 | 0.92 | 2.95 |
| 830 | WH4ug1.XRsdUSG3qXo | CAPZA2 | 8.18 | 9.55 | 0 | 0 | 6.05 | 1 | 2.94 |
| 54602 | W6TMoe6re7uy4i7slE | NDFIP2 | 8.41 | 4.59 | 0 | 0.03 | −0.53 | 0.37 | 2.94 |
| 6477 | lv.m6i7uXm6u7m7_r8 | SIAH1 | 9.8 | 4.44 | 0 | 0.03 | −0.78 | 0.31 | 2.92 |
| 169200 | uXr46X666D.v0lIpx0 | TMEM64 | 7.92 | 11.93 | 0 | 0 | 8.2 | 1 | 2.91 |
| 441454 | lrI4IKoICQmpJ4I44o | LOC441454 | 7.98 | 4.32 | 0 | 0.04 | −0.98 | 0.27 | 2.91 |
| 22934 | Zeqv30z1576CS_FIDk | RPIA | 8.43 | 3.61 | 0 | 0.09 | −2.21 | 0.1 | 2.88 |
| 7178 | ihNxCNaiNmhq_5eiug | TPT1 | 8.11 | 6.04 | 0 | 0.01 | 1.74 | 0.85 | 2.88 |
| 201965 | E7Kr3rjrrF3zxfOwBE | RWDD4A | 8.85 | 5.83 | 0 | 0.01 | 1.43 | 0.81 | 2.86 |
| 51068 | BrKgPKvS.NHoT0opC0 | NMD3 | 7.84 | 10.9 | 0 | 0 | 7.33 | 1 | 2.86 |
| 80777 | fdT.UAsIh1JOl97_VY | CYB5B | 8.26 | 4.68 | 0 | 0.02 | −0.37 | 0.41 | 2.84 |
| | flNOuHtOvcSA5XU7tU | | 8.02 | 4.73 | 0 | 0.02 | −0.3 | 0.43 | 2.82 |
| 115294 | 03JNI0NUINTSQXSFBU | PCMTD1 | 8.09 | 6.44 | 0 | 0 | 2.32 | 0.91 | 2.8 |
| 140890 | 9s.Lqg6Ai_V_QsOCU | SFRS12 | 8.18 | 5.07 | 0 | 0.02 | 0.26 | 0.56 | 2.78 |
| 84061 | BjOAPp6n66dOXutcnE | MAGT1 | 8.43 | 3.57 | 0 | 0.1 | −2.29 | 0.09 | 2.77 |
| 2618 | xU75QpS3gNep0LjXXk | GART | 8.85 | 3.97 | 0 | 0.06 | −1.58 | 0.17 | 2.76 |
| 6902 | rjjjVI_UmSvoJZM.o0 | TBCA | 8.22 | 7.29 | 0 | 0 | 3.45 | 0.97 | 2.74 |
| | WdS75zuQjXd_if.5Mk | | 8.25 | 5.93 | 0 | 0.01 | 1.58 | 0.83 | 2.73 |
| 6670 | KkuP.NQ379QAU7dUqU | SP3 | 8.24 | 5.25 | 0 | 0.01 | 0.54 | 0.63 | 2.73 |
| | 3QAgAvknv_E94OfcI | | 8.32 | 4.63 | 0 | 0.03 | −0.46 | 0.39 | 2.73 |
| | T3rvQHVwCKluPrqpSo | | 10.1 | −3.88 | 0 | 0.07 | −1.74 | 0.15 | 0.37 |
| 10787 | QhwfU65XvpPJHFPsV4 | NCKAP1 | 8.13 | 5.27 | 0 | 0.01 | 0.58 | 0.64 | 2.72 |
| 26065 | TiQsU6QxO7Ie_iO1ak | LSM14A | 8.32 | 7.6 | 0 | 0 | 3.85 | 0.98 | 2.71 |
| 340252 | Hoile8vx.0z4uOTiso | ZNF680 | 7.9 | 5.37 | 0 | 0.01 | 0.74 | 0.68 | 2.68 |
| 262 | Bone.4oUOfCuh.vbAQ | AMD1 | 8.4 | 3.68 | 0 | 0.09 | −2.1 | 0.11 | 2.68 |
| 83930 | NuzpeC76H5AKoICg_Y | STARD3NL | 8.31 | 5.56 | 0 | 0.01 | 1.03 | 0.74 | 2.67 |
| 81688 | lf1UL5z7CLPeebujks | C6orf62 | 8.38 | 4.25 | 0 | 0.04 | −1.1 | 0.25 | 2.67 |
| 85403 | 06QoKyj94B0pfBQyvo | EAF1 | 8.17 | 4.46 | 0 | 0.03 | −0.74 | 0.32 | 2.66 |
| | Tlmcdek7o0UIxD9614 | | 7.97 | 7.46 | 0 | 0 | 3.67 | 0.98 | 2.65 |
| 51020 | HSgmtOAuU6OBHorpLk | HDDC2 | 8.1 | 7.55 | 0 | 0 | 3.78 | 0.98 | 2.65 |
| 648852 | Hp05ew1ST_rrUtPGNE | LOC648852 | 8.34 | 3.6 | 0 | 0.1 | −2.24 | 0.1 | 2.6 |
| 7324 | Newpugyi_dLo_vc77o | UBE2E1 | 8.12 | 3.71 | 0 | 0.08 | −2.04 | 0.12 | 2.6 |
| 10209 | oidAild8WcmK4yhN6c | EIF1 | 8.19 | 6.06 | 0 | 0.01 | 1.77 | 0.85 | 2.59 |
| 51388 | 0SKUYTAD0tX_19VuXU | NIP7 | 8.03 | 9.45 | 0 | 0 | 5.94 | 1 | 2.58 |
| 23658 | Bnr16KIKF4LuDi.X4c | LSM5 | 8.37 | 6.65 | 0 | 0 | 2.61 | 0.93 | 2.58 |
| 134266 | ip0d0fgiqN_u_yx_h4 | GRPEL2 | 8.16 | 4.88 | 0 | 0.02 | −0.04 | 0.49 | 2.58 |
| 53938 | lkpP.i6XjxM9dUSQtY | PPIL3 | 8.11 | 6.84 | 0 | 0 | 2.87 | 0.95 | 2.58 |
| 10724 | BRK4e4BI5V635.TR3I | MGEA5 | 7.87 | 6.79 | 0 | 0 | 2.79 | 0.94 | 2.57 |
| 9554 | BMozoilXp9OIECkpUo | SEC22B | 7.99 | 15.2 | 0 | 0 | 10.49 | 1 | 2.57 |
| 1105 | Qv9W.zupDDtA7nOSUo | CHD1 | 7.71 | 6.65 | 0 | 0 | 2.61 | 0.93 | 2.57 |
| 54477 | Qf3a0j5DtJLx5RHXtI | PLEKHA5 | 8.28 | 5.28 | 0 | 0.01 | 0.59 | 0.64 | 2.57 |
| 220213 | rpMDt6JQX6S8ySiBHs | OTUD1 | 8.13 | 6.79 | 0 | 0 | 2.79 | 0.94 | 2.56 |
| 5359 | ZlKBADSi7rLo8uAt10 | PLSCR1 | 7.77 | 10.02 | 0 | 0 | 6.51 | 1 | 2.56 |
| 80306 | 3qL.LbKXzjDxVmuu7I | MED28 | 7.92 | 8.69 | 0 | 0 | 5.12 | 0.99 | 2.54 |
| 3344 | THlLI4UtELgUfdL5Q0 | FOXN2 | 8.03 | 5.94 | 0 | 0.01 | 1.59 | 0.83 | 2.54 |
| 4698 | 06s0AQhA570qAgvpCc | NDUFA5 | 7.87 | 5.21 | 0 | 0.01 | 0.48 | 0.62 | 2.53 |
| 58517 | KEReiKE.gBHvfFdgV4 | RBM25 | 7.98 | 6.25 | 0 | 0 | 2.05 | 0.89 | 2.52 |
| 51192 | oE2VfefS7gKUbgjnmk | CKLF | 8.52 | 3.89 | 0 | 0.07 | −1.73 | 0.15 | 2.52 |
| 84515 | rS4lENEUNHkdSXqQwI | MCM8 | 8.71 | 3.86 | 0 | 0.07 | −1.77 | 0.15 | 2.51 |
| 23478 | 6.U5SL_7qamTuRRIuQ | SEC11A | 8.07 | 5.32 | 0 | 0.01 | 0.65 | 0.66 | 2.5 |
| | T6HQktdUXyXXgyeDEo | | 8.21 | 4.96 | 0 | 0.02 | 0.08 | 0.52 | 2.5 |
| 10628 | fSUyR.vR7Xu0iR4nUU | TXNIP | 8.22 | 5.98 | 0 | 0.01 | 1.65 | 0.84 | 2.49 |
| 51643 | NovrfJZ5KJX3.e6PTQ | TMBIM4 | 8.06 | 7.05 | 0 | 0 | 3.14 | 0.96 | 2.49 |
| 81853 | BVxIrriTsE4nz4hTTI | TMEM14B | 8.2 | 3.98 | 0 | 0.06 | −1.57 | 0.17 | 2.47 |
| 27020 | iqfBe4fwwnoEtR4OrM | NPTN | 8.2 | 4.77 | 0 | 0.02 | −0.24 | 0.44 | 2.46 |
| 8661 | lrdPglBEgfnnu..fS4 | EIF3A | 7.79 | 4.51 | 0 | 0.03 | −0.67 | 0.34 | 2.45 |
| | 3U8Xo7.7Ueoikn66KU | | 8.34 | 3.67 | 0 | 0.09 | −2.1 | 0.11 | 2.45 |
| 51430 | WtSOIkiMS4gPO43u74 | C1orf9 | 8.06 | 3.72 | 0 | 0.08 | −2.02 | 0.12 | 2.44 |
| 126567 | ihtX7SVv5RE_9d_1Ko | FAM148C | 9.91 | −3.78 | 0 | 0.08 | −1.91 | 0.13 | 0.41 |
| 51065 | itSvnEI0Ua.0lOdIS4 | RPS27L | 8.06 | 8.91 | 0 | 0 | 5.37 | 1 | 2.43 |
| 64065 | oep3NMyEp94y.kHsJI | PERP | 8.05 | 3.86 | 0 | 0.07 | −1.77 | 0.15 | 2.43 |
| 130355 | cdeeAghJHeAknoNlLg | LOC130355 | 8.23 | 8.38 | 0 | 0 | 4.78 | 0.99 | 2.42 |
| 7555 | fqCL4tIUsJW16vX4E4 | CNBP | 7.96 | 4.83 | 0 | 0.02 | −0.14 | 0.47 | 2.42 |
| 84839 | 3_IUl6uESQhVN1EBv8 | RAX2 | 8.36 | 4.84 | 0 | 0.02 | −0.12 | 0.47 | 2.39 |
| 51123 | 0TVTvweER6qdL7uew4 | ZNF706 | 7.78 | 6.85 | 0 | 0 | 2.87 | 0.95 | 2.38 |
| 284996 | NV.Pl0.fuGX76oqA3s | RNF149 | 7.97 | 6.43 | 0 | 0 | 2.31 | 0.91 | 2.37 |
| 84928 | TFuzS7yO5NW.7T1dIc | TMEM209 | 7.87 | 8.41 | 0 | 0 | 4.81 | 0.99 | 2.37 |
| 54830 | oJfvfK.oEt63Xu.Tv0 | NUP62CL | 7.93 | 5.78 | 0 | 0.01 | 1.36 | 0.8 | 2.37 |
| 2353 | cVLhH0iJ6y8sk74lKU | FOS | 7.81 | 6.76 | 0 | 0 | 2.76 | 0.94 | 2.37 |
| 829 | 9gCu.udb.v3nSwzLQk | CAPZA1 | 8.32 | 5.27 | 0 | 0.01 | 0.57 | 0.64 | 2.37 |
| 175 | ud3s4QdxIL7mbn90kk | AGA | 7.76 | 6.85 | 0 | 0 | 2.88 | 0.95 | 2.36 |
| 54965 | ZUnD6Crimoo2fgXqKY | PIGX | 8.05 | 3.89 | 0 | 0.07 | −1.73 | 0.15 | 2.36 |
| 7879 | T9.r6BXpL3nV9co3lU | RAB7A | 7.82 | 8.29 | 0 | 0 | 4.68 | 0.99 | 2.35 |
| 81542 | WQ.ew7Vff3Kd757uDU | TXNDC1 | 8.12 | 4.71 | 0 | 0.02 | −0.34 | 0.42 | 2.34 |
| 6659 | BE4SkcobeX.wpL1vCo | SOX4 | 8.41 | 7.25 | 0 | 0 | 3.41 | 0.97 | 2.34 |
| 142 | uFAn28g7eXx6.VSoKA | PARP1 | 8.15 | 3.95 | 0 | 0.06 | −1.61 | 0.17 | 2.32 |
| 58155 | Bo.eJO5IetycvUoHrk | PTBP2 | 7.95 | 6.96 | 0 | 0 | 3.02 | 0.95 | 2.32 |
| 51317 | 3tLitW4t.uLX7tNvak | PHF21A | 8.07 | 4.7 | 0 | 0.02 | −0.34 | 0.41 | 2.31 |
| 29994 | E7BR7v83Hu77rpe_ik | BAZ2B | 7.67 | 5.43 | 0 | 0.01 | 0.83 | 0.7 | 2.31 |
| 1968 | 391X5316XgEagFItAI | EIF2S3 | 8.47 | 4.07 | 0 | 0.05 | −1.42 | 0.2 | 2.31 |
| 5716 | 0p77i30kV1TsXzNXd4 | PSMD10 | 7.76 | 9.73 | 0 | 0 | 6.23 | 1 | 2.3 |
| 9662 | clf.Luzyjup6.n.cUU | CEP135 | 8.1 | 3.7 | 0 | 0.08 | −2.05 | 0.11 | 2.29 |
| 4154 | lNSX0dSevADvkfNJBU | MBNL1 | 7.99 | 6.13 | 0 | 0.01 | 1.88 | 0.87 | 2.29 |
| | o0jMIgt09l.t_x97vc | | 8.17 | 6.82 | 0 | 0 | 2.83 | 0.94 | 2.28 |
| 79738 | Ql3u3Sd7vJc7vyqKv8 | BBS10 | 7.97 | 6.98 | 0 | 0 | 3.05 | 0.95 | 2.27 |
| 7342 | BX_f27hRO.3sUtHqgI | UBP1 | 8.08 | 5.57 | 0 | 0.01 | 1.04 | 0.74 | 2.27 |
| 23271 | Zr9OhHviQ7EuzrufF4 | CAMSAP1L1 | 7.76 | 6.66 | 0 | 0 | 2.61 | 0.93 | 2.26 |
| 11112 | E6HW0QzrKM1dFB1YR0 | HIBADH | 8.06 | 4.27 | 0 | 0.04 | −1.07 | 0.26 | 2.26 |
| 51014 | rrSTqTOwDtf96_Xdzk | TMED7 | 8.22 | 5.04 | 0 | 0.02 | 0.21 | 0.55 | 2.25 |
| 27075 | WukXoPx7PT7ake1Huk | TSPAN13 | 8.04 | 8.59 | 0 | 0 | 5.02 | 0.99 | 2.24 |
| 23011 | xjq9LrOJIoIjVIyyUs | RAB21 | 7.91 | 5.66 | 0 | 0.01 | 1.17 | 0.76 | 2.24 |
| 58516 | NVfUXd.l7KOx7Oy05E | FAM60A | 7.8 | 6.64 | 0 | 0 | 2.59 | 0.93 | 2.23 |
| | Wu.75CAC_Se.74T3g4 | | 7.9 | 4.06 | 0 | 0.05 | −1.43 | 0.19 | 2.21 |
| 5093 | 3gqTquScgS7K9XQwVc | PCBP1 | 7.88 | 6.79 | 0 | 0 | 2.79 | 0.94 | 2.21 |
| 1871 | T4goqM4KCOn5IsLauk | E2F3 | 8.3 | 3.68 | 0 | 0.09 | −2.1 | 0.11 | 2.2 |
| 255919 | T1zl9FIMH6i65SAci0 | TMEM188 | 8.36 | 6.16 | 0 | 0.01 | 1.91 | 0.87 | 2.2 |
| 6259 | H_Rcgy5zkSJq5_L77Y | RYK | 8.05 | 3.69 | 0 | 0.08 | −2.08 | 0.11 | 2.2 |
| 6767 | TuyL3X92A_Uu7H6fB0 | ST13 | 7.64 | 5.61 | 0 | 0.01 | 1.11 | 0.75 | 2.2 |
| 83940 | 0a7sqouuT0.jrFCgkk | TATDN1 | 7.57 | 7.79 | 0 | 0 | 4.09 | 0.98 | 2.19 |
| 29978 | EuSXgo0uyw5SgLn.xc | UBQLN2 | 7.87 | 6.99 | 0 | 0 | 3.07 | 0.96 | 2.18 |
| 23608 | NHao516.tTcef49fo0 | MKRN1 | 7.67 | 7.92 | 0 | 0 | 4.23 | 0.99 | 2.18 |
| 54534 | uqR7Qfq.CEooT5C9EU | MRPL50 | 7.97 | 6.1 | 0 | 0.01 | 1.84 | 0.86 | 2.17 |
| 7803 | ifq.oxXy6jjsDIpycU | PTP4A1 | 8.43 | 5.89 | 0 | 0.01 | 1.52 | 0.82 | 2.17 |
| 79752 | lTulCXJNOiUgLMl_e0 | ZFAND1 | 8.27 | 10.19 | 0 | 0 | 6.67 | 1 | 2.17 |
| 865 | Efl8JxSPn.lFPpTHu4 | CBFB | 8.17 | 5.44 | 0 | 0.01 | 0.85 | 0.7 | 2.16 |
| 57122 | xnSItd3DnXIUqH4VPI | NUP107 | 7.93 | 7.15 | 0 | 0 | 3.28 | 0.96 | 2.15 |
| | Ngq1B7hzIHur81.Q14 | | 8.09 | 6.39 | 0 | 0 | 2.24 | 0.9 | 2.15 |
| 10577 | NJ1q6evdLr7dO3f.e0 | NPC2 | 8.32 | 4.37 | 0 | 0.04 | −0.89 | 0.29 | 2.14 |
| 55529 | 9oNJ3ZHlTf5L.gAgPk | TMEM55A | 8.08 | 5.65 | 0 | 0.01 | 1.17 | 0.76 | 2.13 |
| 27430 | ul15.zJL86okfgIm7s | MAT2B | 8.03 | 4.1 | 0 | 0.05 | −1.35 | 0.21 | 2.13 |
| 653573 | Bk9InECgygLKsu_j3o | GCUD2 | 7.96 | 4.77 | 0 | 0.02 | −0.23 | 0.44 | 2.12 |
| | KZG_akiZCkxFIAZ7Xs | | 7.99 | 10.51 | 0 | 0 | 6.97 | 1 | 2.12 |
| | uuhgjOlTXIgfUzEfgI | | 8.26 | 6.5 | 0 | 0 | 2.4 | 0.92 | 2.12 |
| 8545 | ZS9NP658t.sKnd7r_E | CGGBP1 | 7.96 | 10.65 | 0 | 0 | 7.1 | 1 | 2.12 |
| 80213 | WUlQOy3l_ALf0nuHko | TM2D3 | 7.75 | 8.34 | 0 | 0 | 4.73 | 0.99 | 2.12 |
| 54928 | 95TfnP9P9Xugjk6_TU | IMPAD1 | 7.72 | 4.35 | 0 | 0.04 | −0.93 | 0.28 | 2.12 |
| 10049 | EXn.T7t4DuJRsu2l54 | DNAJB6 | 8.01 | 5.95 | 0 | 0.01 | 1.61 | 0.83 | 2.12 |
| 663 | QnO7Uqz51Xi7M4ueLI | BNIP2 | 7.93 | 4.89 | 0 | 0.02 | −0.03 | 0.49 | 2.1 |
| 1387 | NqbdUs8V6OMoPh1puQ | CREBBP | 8.15 | 4.06 | 0 | 0.05 | −1.43 | 0.19 | 2.1 |
| 54665 | lP5emhz_b7ul8v78_E | RSBN1 | 7.71 | 6.78 | 0 | 0 | 2.78 | 0.94 | 2.1 |
| 64207 | 3qqOq7P_Qv7iqAO.4 | C14orf4 | 7.83 | 5.15 | 0 | 0.01 | 0.39 | 0.6 | 2.1 |
| 90799 | rV1c4pSH4wyyuCveik | CCDC45 | 7.83 | 7.24 | 0 | 0 | 3.39 | 0.97 | 2.1 |
| 360023 | Wi7p5SAMMQB6k.J71E | ZBTB41 | 7.65 | 6.04 | 0 | 0.01 | 1.75 | 0.85 | 2.09 |
| 3181 | 3.X_koK7fnkrBw4ILo | HNRNPA2B1 | 8.01 | 5.69 | 0 | 0.01 | 1.22 | 0.77 | 2.08 |
| 7082 | ohCqS66.3vf3pzk.gA | TJP1 | 8.47 | 4.59 | 0 | 0.03 | −0.52 | 0.37 | 2.07 |
| 64431 | EafR6Ev9fIKy.7uHuE | ACTR6 | 7.6 | 7.6 | 0 | 0 | 3.85 | 0.98 | 2.07 |
| 9445 | KqFEuHz_y_8vlXnFcs | ITM2B | 7.68 | 9.54 | 0 | 0 | 6.03 | 1 | 2.07 |
| 3183 | ZTw9M_VZly1T.R9f4Y | HNRNPC | 8.11 | 4.64 | 0 | 0.03 | −0.45 | 0.39 | 2.07 |
| 64968 | EDF16j7ictPSXuwU7o | MRPS6 | 8.27 | 3.77 | 0 | 0.08 | −1.93 | 0.13 | 2.06 |
| 51582 | WrkH_LX6fhzEpfgfTo | AZIN1 | 7.76 | 5.75 | 0 | 0.01 | 1.31 | 0.79 | 2.06 |
| 6596 | 6qPJWck8okvqC8XrI0 | HLTF | 7.55 | 5.94 | 0 | 0.01 | 1.6 | 0.83 | 2.06 |
| 64746 | ZVJ0yu9Me8TUgT_0p0 | ACBD3 | 7.76 | 5.4 | 0 | 0.01 | 0.78 | 0.69 | 2.06 |
| 50808 | WunPH_9_tfRKl51NUU | AK3 | 7.89 | 7.68 | 0 | 0 | 3.94 | 0.98 | 2.05 |
| 57182 | xo6ueiOV5fnz_.e6qM | ANKRD50 | 7.7 | 8.77 | 0 | 0 | 5.21 | 0.99 | 2.05 |
| 7852 | QpKF7pQvfL57O3brKE | CXCR4 | 8.17 | 8.41 | 0 | 0 | 4.81 | 0.99 | 2.05 |
| 8531 | 9XkaCEIiyXdS.SQWco | CSDA | 8.26 | 5.26 | 0 | 0.01 | 0.56 | 0.64 | 2.05 |
| | uop775UlKlnk.4QT9Q | | 8.7 | 5.02 | 0 | 0.02 | 0.18 | 0.54 | 2.04 |
| 3838 | 3JNewMBPQtRX.fh9AA | KPNA2 | 7.84 | 4.04 | 0 | 0.06 | −1.45 | 0.19 | 2.04 |
| 4144 | i65p6U6ICeH6eu6xIg | MAT2A | 8.1 | 4.03 | 0 | 0.06 | −1.48 | 0.18 | 2.04 |
| 900 | 6nngsu.KNdTpLy4Owg | CCNG1 | 8.08 | 4.78 | 0 | 0.02 | −0.21 | 0.45 | 2.03 |
| 10627 | ZPwXFJX3VUMHutzEi0 | MRCL3 | 7.83 | 7.15 | 0 | 0 | 3.28 | 0.96 | 2.03 |
| 5412 | ll4OOJc7V5cEeu3R00 | UBL3 | 7.66 | 10.57 | 0 | 0 | 7.02 | 1 | 2.03 |
| 1657 | oNuQwUzmHP_U6IDeEk | DMXL1 | 7.66 | 5.68 | 0 | 0.01 | 1.21 | 0.77 | 2.02 |
| 56889 | 0N70r67FuwLqjqsAus | TM9SF3 | 7.88 | 4.12 | 0 | 0.05 | −1.32 | 0.21 | 2.01 |
| 29116 | 3k.6CetJySv96jtqIU | MYLIP | 7.88 | 6.61 | 0 | 0 | 2.54 | 0.93 | 2.01 |
| 10797 | NqwsZB8f9RQIHtJJ5c | MTHFD2 | 7.72 | 6.25 | 0 | 0 | 2.04 | 0.88 | 2.01 |
| 10728 | Hd8k6KGCAnsT75_It4 | PTGES3 | 8.06 | 3.98 | 0 | 0.06 | −1.56 | 0.17 | 2.01 |
| 90390 | KanpUXb.nLgiKYgPng | MED30 | 7.87 | 9.92 | 0 | 0 | 6.41 | 1 | 2.01 |

TABLE A4
Genes Overexpressed in MDA MB 231 cells (Top Genes of Interest, Fold = MDA.3A2/MDA.IgG)

| LOCUS | | | | | | | | PROB OF | FOLD |
|---|---|---|---|---|---|---|---|---|---|
| LINK ID | ID | GENESYMBOL | AVEEXPR | T | P. VALUE | ADJ. P. VAL | B | DIFF EXP | CHANGE |
| 928 | rWSgWYjrci0nxNXiSg | CD9 | 8.79 | 11.38 | 0 | 0 | 7.77 | 1 | 8.04 |
| 5757 | QQ3z1iT1LB..uzsfJ4 | PTMA | 9.07 | 7.55 | 0 | 0 | 3.62 | 0.97 | 7.15 |
| 7325 | EiHe.NHJfe1dWSCHvo | UBE2E2 | 8.51 | 7.85 | 0 | 0 | 4 | 0.98 | 6.67 |
| 10983 | oioTn1X7UX_SXv3tOw | CCNI | 9.9 | 6.59 | 0 | 0 | 2.31 | 0.91 | 6.24 |
| 50615 | 9uFU4ntnk3904JGqBo | IL21R | 9.95 | −5.21 | 0 | 0 | 0.23 | 0.56 | 0.17 |
| 51192 | oE2VfefS7gKUbgjnmk | CKLF | 8.52 | 7.45 | 0 | 0 | 3.49 | 0.97 | 5.86 |
| 10250 | 3Qf0iXfs.oKegqVIf4 | SRRM1 | 8.89 | 6.99 | 0 | 0 | 2.87 | 0.95 | 5.74 |
| 6789 | B4rSS.s4hMn11PlVHU | STK4 | 8.41 | 8.85 | 0 | 0 | 5.19 | 0.99 | 5.67 |
| 2280 | 9e1epSSoXeCX3_6X.8 | FKBP1A | 8.74 | 5.49 | 0 | 0 | 0.67 | 0.66 | 5.62 |
| | 91epSSoXeCX3_6Xz.8 | | 8.67 | 5.5 | 0 | 0 | 0.7 | 0.67 | 5.53 |
| | 0u8YIQMtNHfU.4qpSo | | 10.05 | −5.55 | 0 | 0 | 0.77 | 0.68 | 0.18 |
| 5376 | xyJ_nq3Ke97l1Ch7ek | PMP22 | 8.64 | 7.38 | 0 | 0 | 3.4 | 0.97 | 5.51 |
| | WiCeQtLnkLSwQPd.6o | | 10.21 | −5.08 | 0 | 0 | 0.01 | 0.5 | 0.19 |
| 5146 | oV7hzggDMz5yVADVKo | PDE6C | 10.28 | −5.18 | 0 | 0 | 0.18 | 0.54 | 0.19 |
| 805 | Kvvgu6L7B3m6HOhLQQ | CALM2 | 9.75 | 5.38 | 0 | 0 | 0.5 | 0.62 | 5.08 |
| 23531 | 6p_X8jaueM_Xv1yw6k | MMD | 8.74 | 8.52 | 0 | 0 | 4.81 | 0.99 | 4.99 |
| 8531 | 9XkaCEIiyXdS.SQWco | CSDA | 8.26 | 11.78 | 0 | 0 | 8.13 | 1 | 4.99 |
| 26511 | WULO_39Q65fn653Xro | CHIC2 | 8.09 | 10.44 | 0 | 0 | 6.88 | 1 | 4.95 |
| 9802 | 0Lt45pR09p1Ug9ch6s | DAZAP2 | 9.11 | 6.05 | 0 | 0 | 1.52 | 0.82 | 4.93 |
| | T3rvQHVwCKluPrqpSo | | 10.1 | −6.14 | 0 | 0 | 1.66 | 0.84 | 0.2 |
| 7570 | fV_F33pPde53hAeJSU | ZNF22 | 8.95 | 5.39 | 0 | 0 | 0.51 | 0.62 | 4.83 |
| 481 | unu3iN6N5U0f6cuEqc | ATP1B1 | 9 | 5.03 | 0 | 0 | −0.07 | 0.48 | 4.82 |
| 144195 | TVKHn3g55x_P3901Rk | SLC2A14 | 9.01 | −7.66 | 0 | 0 | 3.76 | 0.98 | 0.21 |
| | 3jtH4VT87sokcRT6.U | | 8.54 | 6.68 | 0 | 0 | 2.44 | 0.92 | 4.77 |
| | rmU1_i8gtIlEgfEoKo | | 9.87 | −5.27 | 0 | 0 | 0.32 | 0.58 | 0.21 |
| 23471 | rvRN_9VP3R_7RSe.uU | TRAM1 | 8.54 | 6.58 | 0 | 0 | 2.31 | 0.91 | 4.73 |
| 56943 | rh_ungNHUApIlesXhI | ENY2 | 8.7 | 7.32 | 0 | 0 | 3.31 | 0.96 | 4.71 |
| | o0jMIgt09l.t_x97vc | | 8.17 | 12.79 | 0 | 0 | 8.98 | 1 | 4.69 |
| | cV.XdXSD_7_eXc37.8 | | 8.15 | 5.55 | 0 | 0 | 0.76 | 0.68 | 4.64 |
| 54890 | Wd0qlIHX_Um6P3QtqE | ALKBH5 | 8.34 | 4.93 | 0 | 0 | −0.22 | 0.44 | 4.63 |
| 5911 | 3zSS37vJHqwn8PtXyU | RAP2A | 8.55 | 3.59 | 0 | 0.02 | −2.55 | 0.07 | 4.63 |
| 54499 | uYd0KR7s5XkL6e3OJM | TMCO1 | 8.53 | 8.59 | 0 | 0 | 4.9 | 0.99 | 4.62 |
| 56675 | ulCHVOeXT_z0JfwSio | NRIP3 | 9.95 | −5.17 | 0 | 0 | 0.17 | 0.54 | 0.22 |
| | QdMemVgEEgZBADdUqo | | 9.73 | −6.04 | 0 | 0 | 1.52 | 0.82 | 0.22 |
| 23204 | ZKnvriJIfiuOMvpd60 | ARL6IP1 | 8.7 | 6.65 | 0 | 0 | 2.4 | 0.92 | 4.55 |
| 1979 | BslHrteoP3r6P65Xgc | EIF4EBP2 | 8.6 | 10.18 | 0 | 0 | 6.62 | 1 | 4.53 |
| 57092 | uopJ9Ie.z66yefnit8 | PCNP | 8.44 | 6.85 | 0 | 0 | 2.68 | 0.94 | 4.53 |
| | KEU0k28Q8Md.COAd6o | | 10.01 | −5.13 | 0 | 0 | 0.09 | 0.52 | 0.22 |
| 81929 | oOeEeUF1LNdFdkhjN0 | SEH1L | 8.28 | 6.18 | 0 | 0 | 1.72 | 0.85 | 4.53 |
| | BneDXnVKlABNhcgoKo | | 9.76 | −5.46 | 0 | 0 | 0.63 | 0.65 | 0.22 |
| 9349 | 61JLrT2EGAtMWyAI6Y | RPL23 | 8.86 | 4.47 | 0 | 0.01 | −1.01 | 0.27 | 4.49 |
| 80777 | fdT.UAsIh1JOl97_VY | CYB5B | 8.26 | 6.73 | 0 | 0 | 2.51 | 0.92 | 4.48 |
| 1051 | 3h4ZBUbsHtJaleLDZ8 | CEBPB | 8.43 | 10.06 | 0 | 0 | 6.5 | 1 | 4.46 |
| 5376 | BUGRWL_7_KUV4o2oSc | PMP22 | 7.86 | 8.87 | 0 | 0 | 5.22 | 0.99 | 4.45 |
| 493856 | 3GcqHr1KlETMUA3lTE | CISD2 | 8.94 | 7.77 | 0 | 0 | 3.9 | 0.98 | 4.4 |
| 112714 | 9o3uRKHkqfXTfUSf6o | TUBA3E | 9.84 | −4.69 | 0 | 0 | −0.63 | 0.35 | 0.23 |
| 10933 | 355S7.Q46EEioznsi4 | MORF4L1 | 8.99 | 6.24 | 0 | 0 | 1.81 | 0.86 | 4.31 |
| 5094 | c12iGrpOqJJyBDkj00 | PCBP2 | 8.67 | 7.78 | 0 | 0 | 3.91 | 0.98 | 4.29 |
| 83930 | NuzpeC76H5AKoICg_Y | STARD3NL | 8.31 | 8.24 | 0 | 0 | 4.48 | 0.99 | 4.29 |
| 1454 | KUX94ool6LSp6v_jwU | CSNK1E | 8.01 | 8.16 | 0 | 0 | 4.39 | 0.99 | 4.26 |
| 10209 | oidAild8WcmK4yhN6c | EIF1 | 8.19 | 9.17 | 0 | 0 | 5.56 | 1 | 4.23 |
| 7534 | rpFefX_fk1RIc.V01w | YWHAZ | 9.06 | 4.15 | 0 | 0.01 | −1.55 | 0.17 | 4.17 |
| 6205 | K2KmjXd6brhQjgjkig | RPS11 | 8.16 | 6.48 | 0 | 0 | 2.16 | 0.9 | 4.15 |
| | lDUU4IgCSggvQnJV6o | | 9.9 | −5.22 | 0 | 0 | 0.25 | 0.56 | 0.24 |
| 960 | T4Ue8_8f4f9IkRX13o | CD44 | 8.07 | 5.1 | 0 | 0 | 0.05 | 0.51 | 4.12 |
| 81688 | lf1UL5z7CLPeebujks | C6orf62 | 8.38 | 6.13 | 0 | 0 | 1.65 | 0.84 | 4.12 |
| 54602 | W6TMoe6re7uy4i7slE | NDFIP2 | 8.41 | 6.01 | 0 | 0 | 1.47 | 0.81 | 4.09 |
| 56994 | Bo1HMkpFLt_0XodRMo | CHPT1 | 8.17 | 7.09 | 0 | 0 | 3.01 | 0.95 | 4.09 |
| 23576 | 00nHeBI8cKU3XTdwqI | DDAH1 | 8.53 | 5.21 | 0 | 0 | 0.23 | 0.56 | 4.09 |
| | HOIEVCRGCPeSm7Kqho | | 9.54 | −5.49 | 0 | 0 | 0.67 | 0.66 | 0.24 |
| 30968 | N42LQji8uiep_lKi3o | STOML2 | 8.36 | 4.61 | 0 | 0 | −0.76 | 0.32 | 4.08 |
| 2811 | xt9EESkRD9LVJQJIKo | GP1BA | 9.91 | −5.25 | 0 | 0 | 0.29 | 0.57 | 0.24 |
| 7027 | TFXnpoyQh3ui.vS6xo | TFDP1 | 9.31 | 3.47 | 0.01 | 0.02 | −2.75 | 0.06 | 4.08 |
| 125144 | WUCkSlC_yf7V_U05UE | C17orf45 | 8.82 | 4.4 | 0 | 0.01 | −1.12 | 0.25 | 4.05 |
| | 3ndP7d.E5d9HEi3qJc | | 8.95 | −8.34 | 0 | 0 | 4.59 | 0.99 | 0.25 |
| 1968 | 391X5316XgEagFItAI | EIF2S3 | 8.47 | 6.79 | 0 | 0 | 2.6 | 0.93 | 4.04 |
| | QPR6W2.xBr2_AFf.6o | | 9.83 | −5.26 | 0 | 0 | 0.31 | 0.58 | 0.25 |
| 7402 | TFkYRSRo3RorTokj0Q | UTRN | 8.86 | −6.61 | 0 | 0 | 2.35 | 0.91 | 0.25 |
| 10285 | 3Svt5P767C4E00S814 | SMNDC1 | 8.71 | 8.83 | 0 | 0 | 5.17 | 0.99 | 4.02 |
| 51187 | frL7o56o4geDDf5ei4 | C15orf15 | 8.9 | 6.93 | 0 | 0 | 2.79 | 0.94 | 4.01 |
| | WUQEjs7ct7.0ut3W30 | | 7.81 | 15.08 | 0 | 0 | 10.67 | 1 | 4.01 |
| | QpA1IiEgrK.uvnqpyo | | 9.88 | −6.04 | 0 | 0 | 1.52 | 0.82 | 0.25 |
| 6637 | 6p70kiAVO13dTKl7.E | SNRPG | 8.8 | 3.84 | 0 | 0.01 | −2.1 | 0.11 | 3.99 |
| 56650 | cCBv0Uw4V1InqAXFEI | CLDND1 | 8.03 | 13.98 | 0 | 0 | 9.89 | 1 | 3.98 |
| 10577 | NJ1q6evdLr7dO3f.e0 | NPC2 | 8.32 | 7.92 | 0 | 0 | 4.09 | 0.98 | 3.98 |
| 1266 | lul7oT8perjk.nfXv4 | CNN3 | 8.28 | 5.01 | 0 | 0 | −0.1 | 0.48 | 3.95 |
| 4904 | QrnhBSrkogQrIUuKSA | YBX1 | 8.91 | 4.43 | 0 | 0.01 | −1.07 | 0.26 | 3.95 |
| | KSKLCD61eV0KB0S3Ko | | 9.83 | −5.01 | 0 | 0 | −0.1 | 0.47 | 0.25 |
| 3930 | rdek0gLrpv97Ho_RQo | LBR | 8.18 | 5.92 | 0 | 0 | 1.34 | 0.79 | 3.94 |
| 51176 | Qun3e4Pl7BEy6fdZX4 | LEF1 | 7.85 | 29.08 | 0 | 0 | 16.84 | 1 | 3.93 |
| 26065 | TiQsU6QxO7Ie_iO1ak | LSM14A | 8.32 | 10.45 | 0 | 0 | 6.89 | 1 | 3.93 |
| 8655 | u6HuURFS41NSAQoeSU | DYNLL1 | 8.07 | 15.95 | 0 | 0 | 11.24 | 1 | 3.91 |
| | WUSEjs7ct7.0ut3U30 | | 7.9 | 12.24 | 0 | 0 | 8.52 | 1 | 3.9 |
| | KZG_akiZCkxFIAZ7Xs | | 7.99 | 18.98 | 0 | 0 | 12.98 | 1 | 3.9 |
| | 0yK7oA6115Ei2l1I6o | | 9.75 | −5.21 | 0 | 0 | 0.22 | 0.56 | 0.26 |
| 5292 | ro67l9SNd3qnu.4kko | PIM1 | 8.51 | 4.81 | 0 | 0 | −0.43 | 0.39 | 3.89 |
| | lt7HkOEt.q.AySf0Ko | | 9.84 | −4.98 | 0 | 0 | −0.15 | 0.46 | 0.26 |
| 23406 | xIilSrqh6g.CiJ6gaM | COTL1 | 8.06 | 7.74 | 0 | 0 | 3.86 | 0.98 | 3.89 |
| 283412 | u_VFUJ_oJu.uXs_UKo | LOC283412 | 9.7 | −4.94 | 0 | 0 | −0.21 | 0.45 | 0.26 |
| 4277 | HhSf37RfitV2VbRFvM | MICB | 8.15 | 6.25 | 0 | 0 | 1.82 | 0.86 | 3.87 |
| 139886 | Kz54_x6_fvh7HPSOk | SPIN4 | 8.15 | 8.97 | 0 | 0 | 5.33 | 1 | 3.86 |
| 126567 | ihtX7SVv5RE_9d_1Ko | FAM148C | 9.91 | −5.73 | 0 | 0 | 1.04 | 0.74 | 0.26 |
| | cdxTd9OnP6TIz0v5Ko | | 9.87 | −5.18 | 0 | 0 | 0.18 | 0.55 | 0.26 |
| 60 | ZuropJSp8XsR4fiFL4 | ACTB | 10.04 | 2.68 | 0.02 | 0.05 | −4.17 | 0.02 | 3.83 |
| 64089 | cy75e3vcCYFJR.9Dek | SNX16 | 8.36 | 13.01 | 0 | 0 | 9.16 | 1 | 3.82 |
| 114908 | oqeOni_zvHB_leHr7k | TMEM123 | 9 | 4.01 | 0 | 0.01 | −1.8 | 0.14 | 3.81 |
| | 9njAklXtwAPlKdEgKo | | 9.76 | −5.29 | 0 | 0 | 0.35 | 0.59 | 0.27 |
| 10092 | 0YsncAQFF4UIqC4n7k | ARPC5 | 8.31 | 7.62 | 0 | 0 | 3.7 | 0.98 | 3.75 |
| 9791 | fpizEUNKIqyeXkyiXY | PTDSS1 | 8.35 | 5.51 | 0 | 0 | 0.7 | 0.67 | 3.74 |
| 10409 | oSEiVHyftROjdDlDXU | BASP1 | 8.67 | 2.98 | 0.01 | 0.04 | −3.64 | 0.03 | 3.74 |
| 829 | 9gCu.udb.v3nSwzLQk | CAPZA1 | 8.32 | 8.04 | 0 | 0 | 4.24 | 0.99 | 3.72 |
| 441087 | 3_v4Ax_iKWruunRl7o | LOC441087 | 9.64 | 3.33 | 0.01 | 0.02 | −3.02 | 0.05 | 3.71 |
| 7401 | 9EXoQ.cghxD0tyUQKo | CLRN1 | 9.65 | −5.97 | 0 | 0 | 1.41 | 0.8 | 0.27 |
| 6629 | QonlKjn8So.CNEW1Tk | SNRPB2 | 8.19 | 11.17 | 0 | 0 | 7.58 | 1 | 3.7 |
| | 3_dx6HGuKOu4VTM4.0 | | 9.69 | 3.58 | 0 | 0.02 | −2.56 | 0.07 | 3.69 |
| | 0IVAtI4TSsAOSntfKo | | 9.68 | −4.75 | 0 | 0 | −0.52 | 0.37 | 0.27 |
| 10776 | ldJER5S31UM3t13Q9U | ARPP-19 | 8.66 | 5.24 | 0 | 0 | 0.27 | 0.57 | 3.68 |
| 5352 | uEC_Jfn31v_V.t2dc | PLOD2 | 8.51 | 6.35 | 0 | 0 | 1.97 | 0.88 | 3.68 |
| 830 | WH4ug1.XRsdUSG3qXo | CAPZA2 | 8.18 | 11.52 | 0 | 0 | 7.9 | 1 | 3.68 |
| 9617 | Ny6OfOXsNUuf4KlAqo | MTRF1 | 9.47 | −5.87 | 0 | 0 | 1.25 | 0.78 | 0.27 |
| | Qv4vIeZ1OrFJM6XdKo | | 9.59 | −5.23 | 0 | 0 | 0.26 | 0.56 | 0.27 |
| 51241 | Zeggz2KFSl_oI14XXU | C14orf112 | 7.99 | 14.73 | 0 | 0 | 10.43 | 1 | 3.65 |
| 998 | Wiq65yCEhN7oOPRNd0 | CDC42 | 8.18 | 9.1 | 0 | 0 | 5.47 | 1 | 3.65 |
| 285636 | Qjr9I_fl3.d.cXxKnU | LOC285636 | 8.31 | 6.96 | 0 | 0 | 2.83 | 0.94 | 3.64 |
| 90390 | KanpUXb.nLgiKYgPng | MED30 | 7.87 | 18.38 | 0 | 0 | 12.66 | 1 | 3.63 |
| | TSUId6F52K.UCp49Ko | | 9.56 | −5.65 | 0 | 0 | 0.92 | 0.72 | 0.28 |
| 6281 | WpIDiS9nXV4wi_1Aq0 | S100A10 | 8.11 | 10.05 | 0 | 0 | 6.49 | 1 | 3.62 |
| | rht79Y936v7VKLif6o | | 9.66 | −5.13 | 0 | 0 | 0.1 | 0.52 | 0.28 |
| 9334 | urpV8N_3_Pnu5KgK7U | B4GALT5 | 8.17 | 6.16 | 0 | 0 | 1.69 | 0.84 | 3.61 |
| | ug3uVd4Rz6CQOAM26o | | 9.62 | −5.54 | 0 | 0 | 0.74 | 0.68 | 0.28 |
| 30000 | fe56o_mWK6biPUfuyA | TNPO2 | 8.14 | 4.35 | 0 | 0.01 | −1.21 | 0.23 | 3.6 |
| 84522 | fdfiAXJLpTIgsh6s6o | JAGN1 | 9.59 | −5.06 | 0 | 0 | −0.01 | 0.5 | 0.28 |
| 10959 | 9pf6lHvSrHhTS7STyo | TMED2 | 8.77 | 14.08 | 0 | 0 | 9.97 | 1 | 3.6 |
| | u5vR7KXg54wJwU.6ro | | 9.38 | −5.96 | 0 | 0 | 1.4 | 0.8 | 0.28 |
| | ikBfwORQSIrCUJ3Sio | | 9.51 | −5.61 | 0 | 0 | 0.86 | 0.7 | 0.28 |
| 22934 | Zeqv30z1576CS_FIDk | RPIA | 8.43 | 4.34 | 0 | 0.01 | −1.22 | 0.23 | 3.57 |
| 5917 | r7ps55zuCSyee06UKo | RARS | 9.74 | −5.51 | 0 | 0 | 0.71 | 0.67 | 0.28 |
| 6745 | cXSOUqt53JNLCz8kgE | SSR1 | 8.4 | 3.85 | 0 | 0.01 | −2.09 | 0.11 | 3.56 |
| 708 | 0_WFiq6EEf_SOJ66J0 | C1QBP | 8.09 | 18.35 | 0 | 0 | 12.64 | 1 | 3.56 |
| 6431 | Wlx.h.xvVPXu8UX11Y | SFRS6 | 8.76 | 3.87 | 0 | 0.01 | −2.05 | 0.11 | 3.56 |
| 9550 | uF7uCSSUl8Cy1PfnDo | ATP6V1G1 | 8.68 | 9.12 | 0 | 0 | 5.5 | 1 | 3.55 |
| 80790 | 339VXWtUx9Ekd65qCA | CMIP | 8.57 | 4.75 | 0 | 0 | −0.52 | 0.37 | 3.55 |
| 60 | 6EoLV_U1wCUVR93cKI | ACTB | 9.62 | 3.04 | 0.01 | 0.03 | −3.53 | 0.03 | 3.55 |
| | 0VYLNK7XdM9eVXtLoE | | 8.12 | 22.02 | 0 | 0 | 14.39 | 1 | 3.54 |
| 51020 | HSgmtOAuU6OBHorpLk | HDDC2 | 8.1 | 9.77 | 0 | 0 | 6.2 | 1 | 3.54 |
| 2002 | BpCIi6vQCHXqdF7yZ4 | ELK1 | 8 | 5.61 | 0 | 0 | 0.86 | 0.7 | 3.53 |
| 7852 | QpKF7pQvfL57O3brKE | CXCR4 | 8.17 | 14.76 | 0 | 0 | 10.45 | 1 | 3.53 |
| | 0ykhKFjnABGIOokAqo | | 9.43 | −6.02 | 0 | 0 | 1.49 | 0.82 | 0.28 |
| | NSlu0jr49EqfyIrlKo | | 9.65 | −4.81 | 0 | 0 | −0.43 | 0.39 | 0.28 |
| 1476 | 3e78KW7T0IlK62aoQE | CSTB | 8.8 | 3.88 | 0 | 0.01 | −2.02 | 0.12 | 3.51 |
| | faVeJCEsZ9BudCtFCk | | 8.29 | 6.18 | 0 | 0 | 1.73 | 0.85 | 3.5 |
| 1964 | xr_jAxdoTz.r63WIcU | EIF1AX | 7.74 | 10.46 | 0 | 0 | 6.9 | 1 | 3.5 |
| 262 | Bone.4oUOfCuh.vbAQ | AMD1 | 8.4 | 4.67 | 0 | 0 | −0.66 | 0.34 | 3.49 |
| 9991 | uJEnKJd4T7eu.xut70 | ROD1 | 8.62 | 4.01 | 0 | 0.01 | −1.8 | 0.14 | 3.49 |
| 5216 | ixuDqeEfqpSA72uNag | PFN1 | 7.95 | 5.6 | 0 | 0 | 0.84 | 0.7 | 3.49 |
| 58155 | Bo.eJO5IetycvUoHrk | PTBP2 | 7.95 | 10.35 | 0 | 0 | 6.79 | 1 | 3.49 |
| | fH3spPTrMFQhLUhFKo | | 9.62 | −5.2 | 0 | 0 | 0.2 | 0.55 | 0.29 |
| 11065 | 6V04FQT4y1fgRE55Yk | UBE2C | 7.77 | 20.28 | 0 | 0 | 13.62 | 1 | 3.49 |
| 55505 | odbBQHfaHuJXjl._Uk | NOLA3 | 7.75 | 12.13 | 0 | 0 | 8.43 | 1 | 3.48 |
| 3688 | WunOQSd0XGYt8f4vLk | ITGB1 | 8.91 | 3.23 | 0.01 | 0.03 | −3.19 | 0.04 | 3.48 |
| 5089 | iqKTqWqkvd10fsB_7I | PBX2 | 8.28 | 4.45 | 0 | 0.01 | −1.04 | 0.26 | 3.48 |
| | lk19P4S7i_0jzNSC6o | | 9.55 | −5.59 | 0 | 0 | 0.83 | 0.7 | 0.29 |
| | 3U8Xo7.7Ueoikn66KU | | 8.34 | 5.1 | 0 | 0 | 0.05 | 0.51 | 3.47 |
| | 3knSoxPLgCkkHf14qo | | 9.36 | −5.61 | 0 | 0 | 0.86 | 0.7 | 0.29 |
| 23658 | Bnr16KIKF4LuDi.X4c | LSM5 | 8.37 | 8.7 | 0 | 0 | 5.03 | 0.99 | 3.46 |
| 56674 | KRP7ST9a264iknv4nU | TMEM9B | 7.95 | 7.36 | 0 | 0 | 3.37 | 0.97 | 3.45 |
| 2079 | or6rqfoESu7Em57LoI | ERH | 8.04 | 9.86 | 0 | 0 | 6.3 | 1 | 3.43 |
| 10817 | 3bQe4KvXuPm.JldW94 | FRS3 | 7.77 | 9.46 | 0 | 0 | 5.87 | 1 | 3.42 |
| 5537 | T0upGOh1A5dC87MXtU | PPP6C | 8.7 | 7.11 | 0 | 0 | 3.04 | 0.95 | 3.42 |
| 55319 | EIrK.z_6IiL4I8qBS4 | FLJ11184 | 8.05 | 6.94 | 0 | 0 | 2.81 | 0.94 | 3.42 |
| 9528 | BieNPnX3RdeU4x7S8U | TMEM59 | 8.84 | 6.49 | 0 | 0 | 2.18 | 0.9 | 3.4 |
| 27346 | TnRCUz6Vjy6x7eHRu4 | TMEM97 | 8.09 | 6.26 | 0 | 0 | 1.85 | 0.86 | 3.39 |
| | uop775UlKlnk.4QT9Q | | 8.7 | 8.57 | 0 | 0 | 4.87 | 0.99 | 3.39 |
| 28972 | 611OlTc2Wk13QuvF70 | SPCS1 | 7.83 | 14.5 | 0 | 0 | 10.27 | 1 | 3.39 |
| | 9STIfB1fCVITe_oXhU | | 8.14 | 7.28 | 0 | 0 | 3.27 | 0.96 | 3.39 |
| 1808 | ceSnuwk78Xvp_f7u3s | DPYSL2 | 8.06 | 7.44 | 0 | 0 | 3.48 | 0.97 | 3.38 |
| 4082 | BkH9RXlT.7AHktN_hc | MARCKS | 7.81 | 11.21 | 0 | 0 | 7.61 | 1 | 3.38 |
| 220134 | xjqRejB0o6qedECFE0 | C18orf24 | 7.98 | 8.05 | 0 | 0 | 4.24 | 0.99 | 3.37 |
| 2280 | uinqCCq_VI4u6tIiUA | FKBP1A | 7.73 | 14.72 | 0 | 0 | 10.42 | 1 | 3.37 |
| 6230 | ua7eec91ifZDllwoYQ | RPS25 | 8.16 | 7.39 | 0 | 0 | 3.41 | 0.97 | 3.37 |
| 8099 | rSCAiQVFAXBChVYEf0 | CDK2AP1 | 9.55 | 3.44 | 0.01 | 0.02 | −2.81 | 0.06 | 3.37 |
| 6155 | BVxJTneunoPhZxVZAs | RPL27 | 7.88 | 7.76 | 0 | 0 | 3.89 | 0.98 | 3.36 |
| | TtKkQd7r2kgoEM.R6o | | 9.49 | −5.12 | 0 | 0 | 0.08 | 0.52 | 0.3 |
| | 6qjsQDlf8E9QF43Sqo | | 9.4 | −5.34 | 0 | 0 | 0.43 | 0.61 | 0.3 |
| 4673 | lrfoO4ET7_y5zU9d14 | NAP1L1 | 8.09 | 9.46 | 0 | 0 | 5.87 | 1 | 3.36 |
| 677 | l6PUrei.1DvDsBIHpE | ZFP36L1 | 8.64 | 5.86 | 0 | 0 | 1.25 | 0.78 | 3.35 |
| | upama6dEf0ztde_8wk | | 8.65 | 3.73 | 0 | 0.01 | −2.29 | 0.09 | 3.35 |
| 147949 | iher1Df5H1P15XcP6o | ZNF583 | 9.65 | −5.31 | 0 | 0 | 0.39 | 0.6 | 0.3 |
| 11014 | Qk3tfSrnaEg98USQKQ | KDELR2 | 8.57 | 9.05 | 0 | 0 | 5.42 | 1 | 3.34 |
| | cxU7L6icx93Tgt6oCo | | 9.55 | −5.28 | 0 | 0 | 0.34 | 0.58 | 0.3 |
| 7769 | TfVYDkfutJJya3.v6o | ZNF226 | 9.68 | −5.33 | 0 | 0 | 0.42 | 0.6 | 0.3 |
| 139516 | xXzn6VcSILjh4Sif6o | LOC139516 | 9.64 | −5.4 | 0 | 0 | 0.53 | 0.63 | 0.3 |
| | 3TygusgH8DZVIv5e.U | | 7.88 | 11.86 | 0 | 0 | 8.19 | 1 | 3.32 |
| 91663 | T73f_VsrqKuqPnm1yc | MYADM | 7.77 | 11.41 | 0 | 0 | 7.8 | 1 | 3.32 |
| 55450 | KzhYQpF167cHVUCEOI | CAMK2N1 | 7.77 | 11.61 | 0 | 0 | 7.97 | 1 | 3.32 |
| 84061 | BjOAPp6n66dOXutcnE | MAGT1 | 8.43 | 4.19 | 0 | 0.01 | −1.48 | 0.19 | 3.31 |
| 51228 | lcXFu7Pd_GqIjgV9J4 | GLTP | 8.44 | 5.82 | 0 | 0 | 1.19 | 0.77 | 3.31 |
| 53938 | lkpP.i6XjxM9dUSQtY | PPIL3 | 8.11 | 8.64 | 0 | 0 | 4.95 | 0.99 | 3.3 |
| 80025 | iukTtSku4o.nszC_Xk | PANK2 | 7.57 | 22.77 | 0 | 0 | 14.7 | 1 | 3.3 |
| | 0CI6rfnS0FJUp6Lkus | | 9.6 | 2.74 | 0.02 | 0.05 | −4.07 | 0.02 | 3.3 |
| | Eh6UE0eiDid0RHl86o | | 9.62 | −5.5 | 0 | 0 | 0.69 | 0.67 | 0.3 |
| 203547 | Ty5Xhyqij_jueT9CW4 | LOC203547 | 8.99 | 4.01 | 0 | 0.01 | −1.8 | 0.14 | 3.29 |
| 1871 | T4goqM4KCOn5IsLauk | E2F3 | 8.3 | 5.53 | 0 | 0 | 0.74 | 0.68 | 3.29 |
| 23476 | 3lqi4pRQUC0vX1Re78 | BRD4 | 8.11 | 9.29 | 0 | 0 | 5.68 | 1 | 3.27 |
| 51569 | xdez3g64HegkqPu3p0 | UFM1 | 7.68 | 20.57 | 0 | 0 | 13.75 | 1 | 3.27 |
| | xt5p9it.0oN.QDqP6o | | 9.37 | −5.78 | 0 | 0 | 1.12 | 0.75 | 0.31 |
| 134266 | ip0d0fgiqN_u_yx_h4 | GRPEL2 | 8.16 | 6.09 | 0 | 0 | 1.59 | 0.83 | 3.26 |
| 25978 | 3oojO7BevD_o66E.6k | CHMP2B | 8.26 | 6 | 0 | 0 | 1.45 | 0.81 | 3.26 |
| 60436 | QuR4kOirriolOeeD3o | TGIF2 | 7.84 | 7.79 | 0 | 0 | 3.92 | 0.98 | 3.24 |
| 56165 | 6.PHwKxYKClK5Lp0ZU | TDRD1 | 8.35 | 4.2 | 0 | 0.01 | −1.46 | 0.19 | 3.23 |
| 5093 | 3gqTquScgS7K9XQwVc | PCBP1 | 7.88 | 10.05 | 0 | 0 | 6.49 | 1 | 3.23 |
| 7295 | ckjYiQh5.0oJfoZ5K4 | TXN | 7.93 | 12.34 | 0 | 0 | 8.61 | 1 | 3.22 |
| | 0bo8jJx7CXR5.LpO6o | | 9.33 | −5.51 | 0 | 0 | 0.7 | 0.67 | 0.31 |
| | ZkXTfT9DJ9noF6JcKo | | 9.53 | −5.13 | 0 | 0 | 0.1 | 0.53 | 0.31 |
| 55173 | NsgkrU7f_6.XkTf6LI | MRPS10 | 8.08 | 4.36 | 0 | 0.01 | −1.18 | 0.23 | 3.21 |
| 55257 | QpZVLnjojlH9u4eYE8 | C20orf20 | 8.82 | 3.77 | 0 | 0.01 | −2.23 | 0.1 | 3.21 |
| 1058 | xp6k.U0zIXeV9Isl0U | CENPA | 7.8 | 16.86 | 0 | 0 | 11.8 | 1 | 3.21 |
| | 3oREUSoLRwrWb.IZ6o | | 9.34 | −5.25 | 0 | 0 | 0.29 | 0.57 | 0.31 |
| 6009 | WpIADoapJ9EKQt9Odo | RHEB | 8.76 | 5.33 | 0 | 0 | 0.41 | 0.6 | 3.19 |
| 91612 | 6lTzJd4PtX1_L_cOfU | CHURC1 | 8.57 | −7.98 | 0 | 0 | 4.15 | 0.98 | 0.31 |
| 11007 | ciBcmfCid5emepgg6o | CCDC85B | 9.29 | −5.1 | 0 | 0 | 0.05 | 0.51 | 0.31 |
| | cXUEsjvSXVwJKIIoKo | | 9.54 | −5.02 | 0 | 0 | −0.08 | 0.48 | 0.31 |
| 7019 | HlBzpe6NA1JeeXn.zo | TFAM | 8.3 | 6.82 | 0 | 0 | 2.64 | 0.93 | 3.17 |
| | 6UR0b1KSAiOBzqQCqo | | 9.25 | −5.78 | 0 | 0 | 1.12 | 0.75 | 0.32 |
| | chEVEeDbm0BHqQJdKo | | 9.31 | −5.75 | 0 | 0 | 1.08 | 0.75 | 0.32 |
| 29883 | iLt7S.cPpO7Ked7h4I | CNOT7 | 7.74 | 11.05 | 0 | 0 | 7.46 | 1 | 3.17 |
| 148 | 0glJ9W1GHvtfHlQoKo | ADRA1A | 9.42 | −5.59 | 0 | 0 | 0.84 | 0.7 | 0.32 |
| 286451 | E6LRbrU.q4QUuH6gkQ | YIPF6 | 8.16 | 4.01 | 0 | 0.01 | −1.81 | 0.14 | 3.16 |
| 130355 | cdeeAghJHeAknoNlLg | LOC130355 | 8.23 | 10.93 | 0 | 0 | 7.36 | 1 | 3.16 |
| | N0d3JT7.0cCkuCLf6o | | 9.6 | −5.25 | 0 | 0 | 0.29 | 0.57 | 0.32 |
| 54815 | NvUPidSV6.1xU5Lyoc | GATAD2A | 8.25 | 2.96 | 0.01 | 0.04 | −3.68 | 0.02 | 3.14 |
| 81853 | BVxIrriTsE4nz4hTTI | TMEM14B | 8.2 | 5.03 | 0 | 0 | −0.07 | 0.48 | 3.14 |
| 3840 | E6dPsoDXuX1X_JELvk | KPNA4 | 7.59 | 17 | 0 | 0 | 11.88 | 1 | 3.14 |
| 90007 | ZgWnZRXeVrCp5UICTw | MIDN | 7.54 | 8.97 | 0 | 0 | 5.33 | 1 | 3.13 |
| 6391 | 9q4J.q_UeXur_enwKE | SDHC | 7.63 | 11.47 | 0 | 0 | 7.86 | 1 | 3.13 |
| 9167 | fSofivk._JOq5KJf3o | COX7A2L | 8.12 | 11.88 | 0 | 0 | 8.22 | 1 | 3.13 |
| 1452 | 6seQeUgfrPsnnlZ4ck | CSNK1A1 | 8.22 | 3.9 | 0 | 0.01 | −2 | 0.12 | 3.12 |
| | uAAYeVNWrX9Xjq_qS8 | | 7.91 | 3.47 | 0.01 | 0.02 | −2.77 | 0.06 | 3.11 |
| 653226 | B4RV5U.3t.DwUK7yu8 | hCG_1781062 | 8.56 | 4.51 | 0 | 0.01 | −0.93 | 0.28 | 3.11 |
| 51643 | NovrfJZ5KJX3.e6PTQ | TMBIM4 | 8.06 | 8.77 | 0 | 0 | 5.1 | 0.99 | 3.11 |
| | uejXokAOoAJhSEkeao | | 9.35 | −6.38 | 0 | 0 | 2.02 | 0.88 | 0.32 |
| | EVKlYgCJ9QBTt7_qZo | | 9.22 | −5.77 | 0 | 0 | 1.11 | 0.75 | 0.32 |
| 79877 | Nu3z03e73y5Il6Vnvo | DCAKD | 7.85 | 7.21 | 0 | 0 | 3.17 | 0.96 | 3.11 |
| | HfVQt3Oe8kVSTRQEqo | | 9.46 | −5.52 | 0 | 0 | 0.71 | 0.67 | 0.32 |
| 10146 | l693.PjqTkurvH6A6U | G3BP1 | 8.89 | 3.58 | 0 | 0.02 | −2.57 | 0.07 | 3.1 |
| | ENIWkPTaUTggSUvsKo | | 9.57 | −4.69 | 0 | 0 | −0.64 | 0.35 | 0.32 |
| | BU4NKGSV0UlzUYNKEY | | 8.6 | −7.21 | 0 | 0 | 3.17 | 0.96 | 0.32 |
| 6902 | rjjjVI_UmSvoJZM.o0 | TBCA | 8.22 | 8.15 | 0 | 0 | 4.38 | 0.99 | 3.09 |
| 808 | iSUInirCpKym4p7oT8 | CALM3 | 8.74 | 2.85 | 0.02 | 0.04 | −3.88 | 0.02 | 3.09 |
| 55529 | 9oNJ3ZHlTf5L.gAgPk | TMEM55A | 8.08 | 8.39 | 0 | 0 | 4.66 | 0.99 | 3.08 |
| 55233 | ulAekCfwvTST3O69Ik | MOBKL1B | 7.84 | 6.43 | 0 | 0 | 2.09 | 0.89 | 3.08 |
| 83990 | NqPEruJRLl6VPfi.4w | BRIP1 | 8.79 | 3.11 | 0.01 | 0.03 | −3.41 | 0.03 | 3.08 |
| 5558 | lbVIueo8a_4lHuXpf8 | PRIM2 | 9.99 | 2.42 | 0.03 | 0.08 | −4.62 | 0.01 | 3.07 |
| | iUDOnyF_kn.iNICgio | | 9.32 | −5.58 | 0 | 0 | 0.81 | 0.69 | 0.33 |
| 51388 | 0SKUYTAD0tX_19VuXU | NIP7 | 8.03 | 11.14 | 0 | 0 | 7.55 | 1 | 3.06 |
| | cegpOQpAFOngKL0Sio | | 9.37 | −5.37 | 0 | 0 | 0.48 | 0.62 | 0.33 |
| 64083 | HqCKfuFLFDfi.f.e1E | GOLPH3 | 8.15 | 4.74 | 0 | 0 | −0.55 | 0.37 | 3.05 |
| | 6SV301Pr3l.uj0lL6o | | 9.67 | −5.72 | 0 | 0 | 1.03 | 0.74 | 0.33 |
| | rhN6IdevSoAS5qeE80 | | 8.16 | 7.09 | 0 | 0 | 3.01 | 0.95 | 3.05 |
| 55322 | 3eF5TAxUCMwHt9RZVU | C5orf22 | 8.79 | 3.86 | 0 | 0.01 | −2.07 | 0.11 | 3.05 |
| 8749 | rV6EyF1I_wPUv69VKo | ADAM18 | 9.51 | −5.49 | 0 | 0 | 0.68 | 0.66 | 0.33 |
| 4342 | W5TR3ekDhxQkpls9qo | MOS | 9.29 | −5.78 | 0 | 0 | 1.13 | 0.76 | 0.33 |
| 55153 | 3TT_J5fTHr9dJfX0l4 | SDAD1 | 8.32 | 4.89 | 0 | 0 | −0.3 | 0.43 | 3.03 |
| 253558 | 6dwbogOJ9OeKy6_XyI | LYCAT | 7.74 | 10.27 | 0 | 0 | 6.71 | 1 | 3.03 |
| 1054 | fb9UtVPIqfiF_xeBBU | CEBPG | 8.18 | 3.99 | 0 | 0.01 | −1.83 | 0.14 | 3.03 |
| 5870 | x1XT6BAF8B6iBfLVd0 | RAB6A | 8.49 | 3.06 | 0.01 | 0.03 | −3.49 | 0.03 | 3.02 |
| 56951 | xJ6CCltTXt36mhLsf0 | C5orf15 | 8.35 | 7.53 | 0 | 0 | 3.6 | 0.97 | 3.01 |
| 202134 | lhQJKEPn41Si.CFJao | FAM153B | 9.56 | −5.15 | 0 | 0 | 0.14 | 0.53 | 0.33 |
| 55013 | 3hfnCnAg1eASmbT3d4 | CCDC109B | 7.82 | 13.48 | 0 | 0 | 9.52 | 1 | 3.01 |
| | cB8oElZ6CI5EqSJOqI | | 9.11 | −5.93 | 0 | 0 | 1.36 | 0.8 | 0.33 |
| 2683 | i6X_oCNIikiukShLAo | B4GALT1 | 8.04 | 5.23 | 0 | 0 | 0.26 | 0.56 | 3.01 |
| | fgq.Uoebt514ne.ws4 | | 8.54 | 3.78 | 0 | 0.01 | −2.21 | 0.1 | 3.01 |
| 10949 | 3pdOgNX9WN.skU3HpI | HNRNPA0 | 8.01 | 7.34 | 0 | 0 | 3.34 | 0.97 | 3.01 |
| | BKUuOiMrR7ukea6Aio | | 9.4 | −5.22 | 0 | 0 | 0.25 | 0.56 | 0.33 |
| 1633 | HXl0t5.dx7ejw8pJec | DCK | 7.89 | 16.27 | 0 | 0 | 11.44 | 1 | 2.99 |
| | K5.h.pTugiI4pJH1Ko | | 9.53 | −4.6 | 0 | 0 | −0.78 | 0.31 | 0.33 |
| | rnqAkAIk7TrIgR5JKo | | 9.28 | −5.17 | 0 | 0 | 0.15 | 0.54 | 0.33 |
| 3304 | Tiuh76h0KH_ee.1ztM | HSPA1B | 8.04 | 4.19 | 0 | 0.01 | −1.49 | 0.18 | 2.98 |
| | 9Qk7tDNXnL16L3ORKo | | 9.32 | −5.2 | 0 | 0 | 0.21 | 0.55 | 0.34 |
| 3148 | ukAuCSgIKlekpQSnQI | HMGB2 | 7.74 | 13.44 | 0 | 0 | 9.49 | 1 | 2.98 |
| 81671 | Tp55MecDpF3qPpcXqg | TMEM49 | 8.22 | 11.06 | 0 | 0 | 7.47 | 1 | 2.97 |
| 78994 | ilU3vrTU14KhKAXVKQ | PRR14 | 7.76 | 8.56 | 0 | 0 | 4.86 | 0.99 | 2.96 |
| 1163 | lgUgXRN_e9aZUcVCAU | CKS1B | 7.81 | 11.21 | 0 | 0 | 7.61 | 1 | 2.96 |
| 8545 | ZS9NP658t.sKnd7r_E | CGGBP1 | 7.96 | 15.35 | 0 | 0 | 10.85 | 1 | 2.95 |
| | 3QAgAvknv_E94OfcI | | 8.32 | 4.99 | 0 | 0 | −0.14 | 0.47 | 2.95 |
| 400629 | 0veju_l4OnvuefuE6o | FLJ35767 | 9.42 | −5.22 | 0 | 0 | 0.24 | 0.56 | 0.34 |
| 114569 | fengk1X6LlOzC_pzyI | MAL2 | 8.62 | 7.24 | 0 | 0 | 3.21 | 0.96 | 2.94 |
| 1539 | 9IJEft2d75Xp6L0uE4 | CYLC2 | 8.61 | 3.02 | 0.01 | 0.03 | −3.56 | 0.03 | 2.93 |
| 54206 | Nnunn0fI5LiETL671g | ERRFI1 | 8.22 | 3.84 | 0 | 0.01 | −2.1 | 0.11 | 2.93 |
| 7529 | feXBv597jrzPlyQkRU | YWHAB | 8.05 | 5.31 | 0 | 0 | 0.38 | 0.59 | 2.93 |
| 10294 | uAA6n_REOr_ieHqBDo | DNAJA2 | 8.02 | 8 | 0 | 0 | 4.18 | 0.98 | 2.92 |
| | Wu.75CAC_Se.74T3g4 | | 7.9 | 5.5 | 0 | 0 | 0.68 | 0.66 | 2.92 |
| 8905 | fueRH8SSfX9.U6R5cs | AP1S2 | 7.76 | 10.35 | 0 | 0 | 6.79 | 1 | 2.92 |
| 55858 | xFvuS6rcU5D.f0keFU | TMEM165 | 7.65 | 16.68 | 0 | 0 | 11.69 | 1 | 2.92 |
| 51444 | rcYEbgoAknW1EGWfao | RNF138 | 9.52 | −5.37 | 0 | 0 | 0.48 | 0.62 | 0.34 |
| 90488 | cqz_kQ4owHzuFR_Dp4 | C12orf23 | 8.19 | 4.87 | 0 | 0 | −0.33 | 0.42 | 2.92 |
| | WkC6ASkrQbbrTe6r5o | | 9.2 | −5.79 | 0 | 0 | 1.13 | 0.76 | 0.34 |
| 255919 | T1zl9FIMH6i65SAci0 | TMEM188 | 8.36 | 8.34 | 0 | 0 | 4.6 | 0.99 | 2.91 |
| 6319 | Nyg7UU4H4xtbu1SOe0 | SCD | 8.12 | 3.88 | 0 | 0.01 | −2.02 | 0.12 | 2.91 |
| | Tnzt7MoO0S5COeclSk | | 8.61 | 3.97 | 0 | 0.01 | −1.87 | 0.13 | 2.91 |
| 64968 | EDF16j7ictPSXuwU7o | MRPS6 | 8.27 | 5.57 | 0 | 0 | 0.79 | 0.69 | 2.91 |
| 23367 | WigB_03p.miCvUXxnY | LARP1 | 7.98 | 4.95 | 0 | 0 | −0.2 | 0.45 | 2.9 |
| 3017 | Wc8GcY5eBcULedeVQI | HIST1H2BD | 7.71 | 11.31 | 0 | 0 | 7.7 | 1 | 2.9 |
| 10390 | 9iUjWS66IvcO_Hv5KU | CEPT1 | 7.97 | 11.67 | 0 | 0 | 8.03 | 1 | 2.9 |
| 60559 | H5OvxXeLzv33LiTl54 | SPCS3 | 7.97 | 7.29 | 0 | 0 | 3.28 | 0.96 | 2.9 |
| 9112 | Qn_78f.p6JojiqUVbk | MTA1 | 7.7 | 8.69 | 0 | 0 | 5.02 | 0.99 | 2.89 |
| 54830 | oJfvfK.oEt63Xu.Tv0 | NUP62CL | 7.93 | 7.1 | 0 | 0 | 3.03 | 0.95 | 2.89 |
| | EqeASNXjlSPVEB4QKo | | 9.38 | −4.91 | 0 | 0 | −0.27 | 0.43 | 0.35 |
| 6046 | Kleqv0tN1U.rVeh7f4 | BRD2 | 8.24 | 3.17 | 0.01 | 0.03 | −3.29 | 0.04 | 2.88 |
| 51478 | Eyeruuk5k7LVJx0go4 | HSD17B7 | 8.76 | 3.14 | 0.01 | 0.03 | −3.34 | 0.03 | 2.88 |
| | cfA.qS5JzUx3ft7wqo | | 9.29 | −5.76 | 0 | 0 | 1.09 | 0.75 | 0.35 |
| | 9hIC13n19KV_fUfVao | | 9.39 | −5.86 | 0 | 0 | 1.24 | 0.78 | 0.35 |
| 84932 | flAfl.n0dNOSX3q_vk | RAB2B | 7.82 | 9.14 | 0 | 0 | 5.52 | 1 | 2.87 |
| 650832 | EAQeVNWrX9Xjq_6S8U | LOC650832 | 8.04 | 3.17 | 0.01 | 0.03 | −3.3 | 0.04 | 2.87 |
| 22822 | 9BLd1lWRSNCy.oTRXE | PHLDA1 | 7.79 | 12.33 | 0 | 0 | 8.6 | 1 | 2.87 |
| 10552 | WCeLiXWU1JOEB7pYWQ | ARPC1A | 8.03 | 7.29 | 0 | 0 | 3.27 | 0.96 | 2.86 |
| 9278 | QjqxHglXnUSLI_ROio | ZBTB22 | 9.16 | −5.58 | 0 | 0 | 0.81 | 0.69 | 0.35 |
| 647000 | H1.UHcXV0didf1XjSI | LOC647000 | 7.94 | 7.25 | 0 | 0 | 3.23 | 0.96 | 2.86 |
| 27430 | ul15.zJL86okfgIm7s | MAT2B | 8.03 | 5.68 | 0 | 0 | 0.98 | 0.73 | 2.85 |
| 55143 | o7h_frpdPU7uXuXqk4 | CDCA8 | 7.76 | 5.76 | 0 | 0 | 1.09 | 0.75 | 2.85 |
| 3183 | ZTw9M_VZly1T.R9f4Y | HNRNPC | 8.11 | 6.69 | 0 | 0 | 2.46 | 0.92 | 2.85 |
| 7733 | HJXe8Pu7dA.vegCAqo | ZNF180 | 9.34 | −5.61 | 0 | 0 | 0.86 | 0.7 | 0.35 |
| | 9_sE6_JdXSYF596Hao | | 9.37 | −5.88 | 0 | 0 | 1.27 | 0.78 | 0.35 |
| | BZoRmAbhB4ToiBQcao | | 9.42 | −5.37 | 0 | 0 | 0.48 | 0.62 | 0.35 |
| 1460 | lheUFL_5Up3Gv0I1NY | CSNK2B | 7.76 | 10.76 | 0 | 0 | 7.2 | 1 | 2.84 |
| 8323 | KSxf3SE.7IPT9pJ0co | FZD6 | 8.1 | 6.15 | 0 | 0 | 1.68 | 0.84 | 2.84 |
| 6732 | HpJ7I3431KxVOxfz4k | SRPK1 | 7.89 | 5.23 | 0 | 0 | 0.26 | 0.57 | 2.83 |
| 23291 | oVdvX6iOOl.gzkhBf4 | FBXW11 | 8 | 3.83 | 0 | 0.01 | −2.12 | 0.11 | 2.83 |
| | u_M5UdFdhg3lZ.qe64 | | 7.86 | 4 | 0 | 0.01 | −1.82 | 0.14 | 2.83 |
| 27020 | iqfBe4fwwnoEtR4OrM | NPTN | 8.2 | 5.48 | 0 | 0 | 0.66 | 0.66 | 2.82 |
| 23480 | xnG7V6T.K7a_pLTo0o | SEC61G | 7.86 | 7.08 | 0 | 0 | 3 | 0.95 | 2.82 |
| 55326 | uOW2O5Of87.0gCru.o | AGPAT5 | 8.1 | 4.49 | 0 | 0.01 | −0.97 | 0.28 | 2.81 |
| | 9uAVI.JP.rXL.UiNKo | | 9.48 | −4.7 | 0 | 0 | −0.62 | 0.35 | 0.36 |
| 80306 | 3qL.LbKXzjDxVmuu7I | MED28 | 7.92 | 9.6 | 0 | 0 | 6.02 | 1 | 2.81 |
| 81542 | WQ.ew7Vff3Kd757uDU | TXNDC1 | 8.12 | 5.7 | 0 | 0 | 0.99 | 0.73 | 2.81 |
| 9978 | cf5Mg7QnBSm1nH0gi4 | RBX1 | 7.66 | 13.68 | 0 | 0 | 9.67 | 1 | 2.8 |
| 6436 | lOp5eiVeu1eiJuiIKo | SFTPA2B | 9.26 | −5.35 | 0 | 0 | 0.45 | 0.61 | 0.36 |
| 2069 | iTe5WP8s7kqqY3qDS0 | EREG | 7.56 | 18.46 | 0 | 0 | 12.7 | 1 | 2.8 |
| 389792 | NmX.d31AMC1CHoi5qo | IER5L | 9.25 | −5.21 | 0 | 0 | 0.22 | 0.56 | 0.36 |
| 388951 | ZrncpKgB4EAKSA49Ko | TSPYL6 | 9.14 | −5.41 | 0 | 0 | 0.55 | 0.64 | 0.36 |
| 3020 | fmSc2uf5KKQuKXN6_k | H3F3A | 7.71 | 9.83 | 0 | 0 | 6.26 | 1 | 2.8 |
| | o3u4r4MI17t1Iit46o | | 9.27 | −5.47 | 0 | 0 | 0.65 | 0.66 | 0.36 |
| | WrgWV_HwGUETcAqpio | | 9.08 | −5.61 | 0 | 0 | 0.86 | 0.7 | 0.36 |
| 6613 | Kf.7Gye8TsqQ3t.Cyo | SUMO2 | 9.7 | 6.33 | 0 | 0 | 1.94 | 0.87 | 2.79 |
| 23568 | x99e0A3dcUI671HtSU | ARL2BP | 7.91 | 4.24 | 0 | 0.01 | −1.4 | 0.2 | 2.79 |
| 8799 | fXfXV87cXRQXZ00.pU | PEX11B | 8.28 | 7.43 | 0 | 0 | 3.47 | 0.97 | 2.79 |
| 6421 | BI_6Dq7CEPrKq4C6v4 | SFPQ | 8.42 | 4.18 | 0 | 0.01 | −1.5 | 0.18 | 2.79 |
| 10276 | ZqbssL7IKdiqG6Hz_U | NET1 | 8.25 | 4.36 | 0 | 0.01 | −1.2 | 0.23 | 2.78 |
| | cUr8U3R1SwxUnXNwJU | | 7.89 | 5.58 | 0 | 0 | 0.81 | 0.69 | 2.78 |
| 827 | iUh10h.3dL6jn0dnqo | CAPN6 | 9.18 | −5.86 | 0 | 0 | 1.25 | 0.78 | 0.36 |
| 51765 | 09tew3v3K4JMdS7.Ko | RP6-213H19.1 | 9.51 | −4.78 | 0 | 0 | −0.48 | 0.38 | 0.36 |
| 3576 | 3Vy3nJSjUQtfvUe5fo | IL8 | 7.47 | 14.82 | 0 | 0 | 10.49 | 1 | 2.77 |
| | ZutQKQL2oQnQwfLfv4 | | 8.24 | 5.47 | 0 | 0 | 0.64 | 0.65 | 2.77 |
| 6747 | xlHI6S5ezI6oAD5RT0 | SSR3 | 7.69 | 16.47 | 0 | 0 | 11.57 | 1 | 2.77 |
| | N7e01L5L_G7eiOoS6o | | 9.08 | −5.18 | 0 | 0 | 0.19 | 0.55 | 0.36 |
| 114882 | NXkuwsSRHtA9.18K5E | OSBPL8 | 7.85 | 7.23 | 0 | 0 | 3.2 | 0.96 | 2.76 |
| 29978 | EuSXgo0uyw5SgLn.xc | UBQLN2 | 7.87 | 9.1 | 0 | 0 | 5.48 | 1 | 2.76 |
| 161742 | BvwUf55wj.ltU9U..U | SPRED1 | 8.01 | 5.48 | 0 | 0 | 0.65 | 0.66 | 2.76 |
| | WUZmAPaUq92d_2bedA | | 7.64 | 15.91 | 0 | 0 | 11.22 | 1 | 2.76 |
| 25907 | EgX.UL43NS4bpeupv0 | TMEM158 | 8.2 | 3.35 | 0.01 | 0.02 | −2.98 | 0.05 | 2.76 |
| 6752 | TXbMJ9CXRAX3Jd5X6o | SSTR2 | 9.43 | −5.76 | 0 | 0 | 1.09 | 0.75 | 0.36 |
| | 3NDg8gVCdQkNdcg.Ko | | 9.11 | −5.92 | 0 | 0 | 1.33 | 0.79 | 0.36 |
| | Tse_fo5pEuvrDoMCjk | | 8.48 | 2.48 | 0.03 | 0.07 | −4.51 | 0.01 | 2.75 |
| | No9r9q9Lg0qS7.UP6o | | 9.19 | −5.26 | 0 | 0 | 0.31 | 0.58 | 0.36 |
| 5270 | Qt_chCvnvuS717Rx60 | SERPINE2 | 7.98 | 7.96 | 0 | 0 | 4.13 | 0.98 | 2.75 |
| 11112 | E6HW0QzrKM1dFB1YR0 | HIBADH | 8.06 | 5.3 | 0 | 0 | 0.38 | 0.59 | 2.75 |
| 440275 | 6OjTeJdKK_KeS4nuHk | EIF2AK4 | 8.33 | 3.26 | 0.01 | 0.03 | −3.13 | 0.04 | 2.75 |
| 83641 | Eqlesp1.IKFP3vXInw | FAM107B | 8.18 | 4.43 | 0 | 0.01 | −1.07 | 0.25 | 2.74 |
| 440026 | rRCCEJN7rmikJKcHKI | TMEM41B | 7.83 | 15.02 | 0 | 0 | 10.63 | 1 | 2.74 |
| 51112 | QBBnlu7edxepCdCh1c | TTC15 | 8.25 | −6.38 | 0 | 0 | 2.02 | 0.88 | 0.36 |
| 56203 | fVyA8gDQn_pSua6dOU | LMOD3 | 8.46 | 3.78 | 0 | 0.01 | −2.2 | 0.1 | 2.74 |
| | BV69MIpw5ItEtJU16o | | 9.23 | −5.24 | 0 | 0 | 0.28 | 0.57 | 0.36 |
| | QL0UFXqNL6nEu.dgqo | | 9.1 | −5.95 | 0 | 0 | 1.38 | 0.8 | 0.37 |
| 10413 | Nro.zXZCTvvMIPuruU | YAP1 | 8.31 | 5.01 | 0 | 0 | −0.1 | 0.48 | 2.74 |
| 9525 | 9q37X0X.C9KgS.teFE | VPS4B | 7.75 | 8.8 | 0 | 0 | 5.14 | 0.99 | 2.74 |
| | Kr6LkumY3SeHklXn1Q | | 9.16 | 2.68 | 0.02 | 0.05 | −4.17 | 0.02 | 2.74 |
| 11165 | c3giKUEl9V5fo94LvU | NUDT3 | 8.34 | 3.97 | 0 | 0.01 | −1.87 | 0.13 | 2.74 |
| 9554 | BMozoilXp9OIECkpUo | SEC22B | 7.99 | 16.2 | 0 | 0 | 11.4 | 1 | 2.74 |
| 3460 | KCF62234f6QOJaJV_o | IFNGR2 | 8.27 | 9.91 | 0 | 0 | 6.35 | 1 | 2.73 |
| 7334 | cavkF_3fkvwrDoz7JU | UBE2N | 8.01 | 11.32 | 0 | 0 | 7.72 | 1 | 2.73 |
| 64783 | Qp0RJ8Isnt3QG5nRL4 | RBM15 | 8 | 4.87 | 0 | 0 | −0.33 | 0.42 | 2.73 |
| | Kf8ECp.VU9oJL6QKpo | | 9.02 | −5.59 | 0 | 0 | 0.83 | 0.7 | 0.37 |
| 140609 | 9phS.dEg_4JeClCKC0 | NEK7 | 7.6 | 13.94 | 0 | 0 | 9.86 | 1 | 2.73 |
| 79192 | ESV1bWXjwq33.H.mqo | IRX1 | 9.16 | −6 | 0 | 0 | 1.45 | 0.81 | 0.37 |
| 51727 | Nyg4vfNy75KUkDEius | CMPK1 | 8.18 | 9.02 | 0 | 0 | 5.39 | 1 | 2.71 |
| 278 | urSS3eNbkqzw7tKni4 | AMY1C | 7.97 | 7.23 | 0 | 0 | 3.2 | 0.96 | 2.71 |
| 4673 | 9HL.3lJzvg5ED3tGTs | NAP1L1 | 7.65 | 12.49 | 0 | 0 | 8.73 | 1 | 2.71 |
| 378 | xVcT4delxNJQHwX66o | ARF4 | 8.42 | 7.25 | 0 | 0 | 3.22 | 0.96 | 2.71 |
| | o_j5N4OSB.rbUsVI78 | | 8.23 | −7.97 | 0 | 0 | 4.14 | 0.98 | 0.37 |
| | EotPMuzV4i3i1Liv6o | | 9.32 | −5.07 | 0 | 0 | 0 | 0.5 | 0.37 |
| | Te_ciWQc7Kyix76fao | | 9.29 | −5.55 | 0 | 0 | 0.77 | 0.68 | 0.37 |
| 3682 | QgfoiTSgpUngNIg3nY | ITGAE | 7.73 | 17.67 | 0 | 0 | 12.27 | 1 | 2.7 |
| | EHOtPME0r_IAH03K6o | | 9.24 | −5.4 | 0 | 0 | 0.54 | 0.63 | 0.37 |
| 79027 | EIl0v7koRYToEqHR6o | ZNF655 | 9.18 | −5.82 | 0 | 0 | 1.19 | 0.77 | 0.37 |
| 91746 | TPXO9LJuvjnPvyX1XU | YTHDC1 | 8.28 | 7.42 | 0 | 0 | 3.44 | 0.97 | 2.7 |
| | HqDQ14t.5FKS0.qJ6o | | 9.06 | −5.48 | 0 | 0 | 0.66 | 0.66 | 0.37 |
| 5037 | os7R3rV.AiQ16CCSio | PEBP1 | 9.13 | −5.43 | 0 | 0 | 0.57 | 0.64 | 0.37 |
| 7358 | EVK3TX0oP_S9DiCKiE | UGDH | 8.1 | 5.98 | 0 | 0 | 1.43 | 0.81 | 2.69 |
| 688 | KboBEXE4AkgNr57.n0 | KLF5 | 7.92 | 6.86 | 0 | 0 | 2.7 | 0.94 | 2.69 |
| 7323 | 067qL7Tinu_C4fNzuo | UBE2D3 | 8.86 | 2.91 | 0.01 | 0.04 | −3.76 | 0.02 | 2.68 |
| 64332 | N_flnpOXig7P6f48RE | NFKBIZ | 8.21 | 7.22 | 0 | 0 | 3.18 | 0.96 | 2.68 |
| 6428 | TVHvFPvfNU6gkvrxGM | SFRS3 | 8.01 | 6.87 | 0 | 0 | 2.71 | 0.94 | 2.68 |
| 5111 | umjOoR8Axx_nVCNijg | PCNA | 7.83 | 8.79 | 0 | 0 | 5.12 | 0.99 | 2.68 |
| 349334 | HE.BExMApLAhA4giqo | FOXD4L4 | 9.12 | −5.89 | 0 | 0 | 1.29 | 0.78 | 0.37 |
| 6670 | KkuP.NQ379QAU7dUqU | SP3 | 8.24 | 5.14 | 0 | 0 | 0.12 | 0.53 | 2.68 |
| 6636 | 3sl5BPaW6.E4vxV0NU | SNRPF | 7.63 | 14.52 | 0 | 0 | 10.29 | 1 | 2.68 |
| 25798 | N1X_r0.nv_X4oJhjlU | BRI3 | 7.58 | 12.41 | 0 | 0 | 8.67 | 1 | 2.68 |
| 55793 | xX16r_gMAfelqRrp0c | FAM63A | 8.21 | 3.83 | 0 | 0.01 | −2.12 | 0.11 | 2.68 |
| 91272 | iykwoB2.kp3e1Knn14 | FAM44B | 8.2 | 4.8 | 0 | 0 | −0.44 | 0.39 | 2.67 |
| 9141 | 3lQvejS2lalK8LB8kc | PDCD5 | 7.73 | 8.08 | 0 | 0 | 4.29 | 0.99 | 2.67 |
| 81848 | 9l.eEv7tglUud5egF4 | SPRY4 | 8.31 | −8.19 | 0 | 0 | 4.42 | 0.99 | 0.37 |
| 7851 | WcT.1XXqv5qhKhIHEo | MALL | 7.6 | 12.22 | 0 | 0 | 8.5 | 1 | 2.67 |
| 3837 | Z0g_kkQIAZXdeH7Puk | KPNB1 | 8.2 | 3.65 | 0 | 0.02 | −2.43 | 0.08 | 2.67 |
| 1607 | WebrTShiQt3lDkD46o | DGKB | 9.07 | −5.56 | 0 | 0 | 0.79 | 0.69 | 0.38 |
| 1716 | fSVd01eiR3lnQiev7w | DGUOK | 7.84 | 7.92 | 0 | 0 | 4.08 | 0.98 | 2.66 |
| 9477 | rJd5BC0rrrTv1fcv_k | MED20 | 7.61 | 17.37 | 0 | 0 | 12.1 | 1 | 2.66 |
| 154043 | 05P_F70u3t9SRruxH0 | CNKSR3 | 7.53 | 11.79 | 0 | 0 | 8.14 | 1 | 2.66 |
| 2504 | oiSuCU0CDquFG4UH7k | FTHL12 | 9.3 | 2.34 | 0.04 | 0.08 | −4.76 | 0.01 | 2.66 |
| 25801 | xvrrv4q_nIDgJej.uU | GCA | 8.48 | 11.18 | 0 | 0 | 7.59 | 1 | 2.66 |
| 80143 | lS60Hq9HTQdPVtUvx0 | SIKE | 8.38 | 3.15 | 0.01 | 0.03 | −3.33 | 0.03 | 2.65 |
| | WeKEgtu.QstKLjqiuo | | 8.96 | −5.45 | 0 | 0 | 0.6 | 0.65 | 0.38 |
| 1479 | QkaCHzgZfvlS9VL3pI | CSTF3 | 7.82 | 10.69 | 0 | 0 | 7.13 | 1 | 2.65 |
| | oXqToNe6TUQBxfQdTI | | 7.82 | 11.28 | 0 | 0 | 7.68 | 1 | 2.65 |
| 200845 | KpLihSCgv_5.pIddTs | KCTD6 | 7.79 | 13.39 | 0 | 0 | 9.45 | 1 | 2.65 |
| 283651 | 6S7tKQ3of5XQCJLr14 | C15orf21 | 7.69 | 16.9 | 0 | 0 | 11.82 | 1 | 2.65 |
| 58516 | NVfUXd.l7KOx7Oy05E | FAM60A | 7.8 | 8.08 | 0 | 0 | 4.29 | 0.99 | 2.65 |
| 8763 | TvI.5C3EDid_QSB0SU | CD164 | 8.26 | 8.63 | 0 | 0 | 4.94 | 0.99 | 2.65 |
| | Q3Sfd1WEXo4dd_nF6o | | 9.22 | −5.95 | 0 | 0 | 1.39 | 0.8 | 0.38 |
| 441394 | E0g6dIN4ri3R3tlSno | SUGT1P | 8.85 | 2.72 | 0.02 | 0.05 | −4.1 | 0.02 | 2.65 |
| 6780 | rBUoS.S45A_7ld69J4 | STAU1 | 7.64 | 18.49 | 0 | 0 | 12.72 | 1 | 2.65 |
| | oVIueo8S_4lHuXrf38 | | 9.45 | 2.3 | 0.04 | 0.09 | −4.82 | 0.01 | 2.65 |
| 3777 | 6MeKXoSuC4UiJAKQKo | KCNK3 | 9.13 | −5.26 | 0 | 0 | 0.32 | 0.58 | 0.38 |
| 65979 | 9XicfvZfe5tFCw3r00 | PHACTR4 | 7.83 | 9.09 | 0 | 0 | 5.46 | 1 | 2.64 |
| | cCu0fl2v1UQKuIgoqo | | 8.92 | −6.09 | 0 | 0 | 1.59 | 0.83 | 0.38 |
| | ZW.HUer7gj0SCOEoKo | | 9.14 | −5.98 | 0 | 0 | 1.43 | 0.81 | 0.38 |
| | 9CLKLvRLl_6K0viIuo | | 9.23 | −4.64 | 0 | 0 | −0.71 | 0.33 | 0.38 |
| 51012 | fmuqCBCTDrKqKfpeAc | SLMO2 | 7.9 | 8.66 | 0 | 0 | 4.98 | 0.99 | 2.64 |
| 3075 | uSUg6gOcPSddSklQ6o | CFH | 9.09 | −5.47 | 0 | 0 | 0.64 | 0.66 | 0.38 |
| 651771 | 64Va9Z4jSCkEneL1Ko | LOC651771 | 9.17 | −5.1 | 0 | 0 | 0.05 | 0.51 | 0.38 |
| 93380 | 0k_6fdLp_Ek316_zuk | TMEM32 | 7.99 | 8.98 | 0 | 0 | 5.34 | 1 | 2.63 |
| 51301 | rf_uTx_tD0q_KIe_qo | GCNT4 | 8.91 | −5.5 | 0 | 0 | 0.69 | 0.67 | 0.38 |
| 8533 | NFN_lLhC72ipJIihQk | COPS3 | 7.59 | 8.63 | 0 | 0 | 4.94 | 0.99 | 2.63 |
| | lkn6jt4IOAJcIhClao | | 9.22 | −5.37 | 0 | 0 | 0.48 | 0.62 | 0.38 |
| | cm4lFmVpfyDn8P93SI | | 8.83 | 2.87 | 0.02 | 0.04 | −3.83 | 0.02 | 2.63 |
| 9588 | i6dUKK7JK7iQzy5TuU | PRDX6 | 7.72 | 7.28 | 0 | 0 | 3.26 | 0.96 | 2.63 |
| 5962 | KevBfguHvsU6_5E59I | RDX | 7.66 | 10.16 | 0 | 0 | 6.6 | 1 | 2.63 |
| 7132 | lioZzl05W.6u10UkKc | TNFRSF1A | 7.73 | 6.93 | 0 | 0 | 2.8 | 0.94 | 2.63 |
| 26097 | QOmSEjVgFDj4JeVNdU | C1orf77 | 7.95 | 5.14 | 0 | 0 | 0.11 | 0.53 | 2.63 |
| 10434 | udS5Rqj7hX_vVeO.r0 | LYPLA1 | 7.97 | 14.22 | 0 | 0 | 10.07 | 1 | 2.62 |
| 55811 | 9Dul314ESX5p3eARKo | ADCY10 | 9.24 | −4.9 | 0 | 0 | −0.28 | 0.43 | 0.38 |
| 54806 | fi6R4weuCXElNZUAt4 | AHI1 | 7.96 | 9.75 | 0 | 0 | 6.18 | 1 | 2.62 |
| 55795 | Hm4Kedp0f9cLuvXgp4 | PCID2 | 7.78 | 5.78 | 0 | 0 | 1.12 | 0.75 | 2.62 |
| 1761 | uljpr0fosEp89R.U6o | DMRT1 | 9.34 | −4.75 | 0 | 0 | −0.53 | 0.37 | 0.38 |
| | Wpe5wM16M_.MrSAe6o | | 9.04 | −5.49 | 0 | 0 | 0.68 | 0.66 | 0.38 |
| 10728 | Hd8k6KGCAnsT75_It4 | PTGES3 | 8.06 | 5.47 | 0 | 0 | 0.64 | 0.66 | 2.61 |
| 10252 | xl9s.uCh0lF9ffRVOc | SPRY1 | 7.71 | 16.92 | 0 | 0 | 11.84 | 1 | 2.61 |
| 84436 | rXqdCS195fJdcLJ4hw | ZNF528 | 8.67 | 2.91 | 0.01 | 0.04 | −3.76 | 0.02 | 2.61 |
| 6184 | iSPq6y6ivuTopS4CeE | RPN1 | 7.94 | 4.34 | 0 | 0.01 | −1.22 | 0.23 | 2.61 |
| 55069 | BPSA_tQw5G5.lerElI | C7orf42 | 7.73 | 14.42 | 0 | 0 | 10.21 | 1 | 2.6 |
| 865 | Efl8JxSPn.lFPpTHu4 | CBFB | 8.17 | 6.77 | 0 | 0 | 2.57 | 0.93 | 2.6 |
| 7288 | HUlHA4EotnHi28UG6o | TULP2 | 9.21 | −4.88 | 0 | 0 | −0.31 | 0.42 | 0.38 |
| | 3lEvo9eFRIBjiMfp6o | | 9.08 | −6.73 | 0 | 0 | 2.52 | 0.93 | 0.38 |
| 3187 | ipuBoOoq_3JO1LOLro | HNRPH1 | 7.76 | 10.91 | 0 | 0 | 7.34 | 1 | 2.6 |
| 11157 | oTSxEgiGjuCEUIiQZ8 | LSM6 | 8.17 | 11.96 | 0 | 0 | 8.28 | 1 | 2.6 |
| 8428 | 6md23hP7_g25R563mU | STK24 | 8.45 | 3.31 | 0.01 | 0.02 | −3.05 | 0.05 | 2.59 |
| 83698 | ZUyU31dRF4_dB5VIKo | CALN1 | 9.19 | −5.3 | 0 | 0 | 0.37 | 0.59 | 0.39 |
| 10621 | QlO4CoghATUYFKC6tc | POLR3F | 7.89 | 10.04 | 0 | 0 | 6.48 | 1 | 2.59 |
| 1964 | BKTGmJ9SJOcA0OAeMQ | EIF1AX | 7.71 | 10.49 | 0 | 0 | 6.93 | 1 | 2.59 |
| 51726 | 3F3V.qj8O3rnll4v0I | DNAJB11 | 7.93 | 7.84 | 0 | 0 | 3.98 | 0.98 | 2.59 |
| 55954 | W1_p0JzXVF0l3QMnqE | ZMAT5 | 8.87 | 2.29 | 0.04 | 0.09 | −4.84 | 0.01 | 2.59 |
| | 9mu.guECnnuEACSoCo | | 9.06 | −5.59 | 0 | 0 | 0.84 | 0.7 | 0.39 |
| | BRuDqROl1RJCjnbw6o | | 9.08 | −4.97 | 0 | 0 | −0.17 | 0.46 | 0.39 |
| 54477 | Qf3a0j5DtJLx5RHXtI | PLEKHA5 | 8.28 | 5.31 | 0 | 0 | 0.39 | 0.6 | 2.58 |
| 374969 | rH0evXVN4PpROfngiE | CCDC23 | 7.61 | 15.7 | 0 | 0 | 11.08 | 1 | 2.58 |
| | umSgQB1IKiFoIpAxqo | | 9.02 | −6.05 | 0 | 0 | 1.53 | 0.82 | 0.39 |
| | r4ASIJ7xEzKirMQoCs | | 7.75 | 8.42 | 0 | 0 | 4.69 | 0.99 | 2.58 |
| 127933 | Ev0skz636T9_ke6954 | UHMK1 | 7.83 | 4.98 | 0 | 0 | −0.15 | 0.46 | 2.58 |
| 23314 | HkuvcLg3upP8l7rflI | SATB2 | 7.72 | 10.96 | 0 | 0 | 7.39 | 1 | 2.58 |
| | B0GDC3U_1LTpPR3l6o | | 9.24 | −4.92 | 0 | 0 | −0.24 | 0.44 | 0.39 |
| 5354 | KQFLMQjCfUp5Mgooio | PLP1 | 8.86 | −6.87 | 0 | 0 | 2.71 | 0.94 | 0.39 |
| 7329 | l9dneo5KTn3RbkieR8 | UBE2I | 7.68 | 7.21 | 0 | 0 | 3.17 | 0.96 | 2.57 |
| 6659 | BE4SkcobeX.wpL1vCo | SOX4 | 8.41 | 8.06 | 0 | 0 | 4.26 | 0.99 | 2.57 |
| 131566 | QRf5Xp3k_0XIiQitzI | DCBLD2 | 7.84 | 5.61 | 0 | 0 | 0.86 | 0.7 | 2.57 |
| 6541 | EdV._eEEe7E_FH1xTE | SLC7A1 | 8.53 | 2.77 | 0.02 | 0.05 | −4.01 | 0.02 | 2.57 |
| 4247 | foB4JJCLleDuO93V6o | MGAT2 | 9.5 | −4.61 | 0 | 0 | −0.77 | 0.32 | 0.39 |
| | WSEgO1eV0aL8TXqy6o | | 9.01 | −4.71 | 0 | 0 | −0.6 | 0.36 | 0.39 |
| | WdS75zuQjXd_if.5Mk | | 8.25 | 5.56 | 0 | 0 | 0.79 | 0.69 | 2.57 |
| 54534 | uqR7Qfq.CEooT5C9EU | MRPL50 | 7.97 | 7.43 | 0 | 0 | 3.46 | 0.97 | 2.57 |
| | lS303h3r11910gigYU | | 7.62 | 14.82 | 0 | 0 | 10.5 | 1 | 2.57 |
| 51124 | ElRCJLXAQ0Q8R7HFQg | IER3IP1 | 7.88 | 9.22 | 0 | 0 | 5.61 | 1 | 2.56 |
| 10635 | lV.9_MUdNRu9ePtRP0 | RAD51AP1 | 7.82 | 15.28 | 0 | 0 | 10.8 | 1 | 2.56 |
| 346007 | x7ukPqP5KN4KYAuUKo | EGFL11 | 8.99 | −5.04 | 0 | 0 | −0.05 | 0.49 | 0.39 |
| | uxNAlzoyMKggPygOqo | | 9.01 | −5.23 | 0 | 0 | 0.27 | 0.57 | 0.39 |
| 1112 | xKn6Ovgd7Hl_7kfSVY | FOXN3 | 7.68 | 12.03 | 0 | 0 | 8.35 | 1 | 2.56 |
| 51201 | WoSj2PCz1d_0B6Ccqg | ZDHHC2 | 7.98 | 2.82 | 0.02 | 0.04 | −3.92 | 0.02 | 2.56 |
| 93081 | xR6PiCkDT1OkuAoa2E | C13orf27 | 7.91 | 8.92 | 0 | 0 | 5.27 | 0.99 | 2.56 |
| 6715 | 0TGiwnneXu4unXqVwE | SRD5A1 | 7.58 | 17.22 | 0 | 0 | 12.01 | 1 | 2.56 |
| | iV_kEf0YN.RpXU_mRM | | 8.24 | −8.11 | 0 | 0 | 4.32 | 0.99 | 0.39 |
| 7295 | obSHUklCOuCSNiJCHk | TXN | 7.75 | 11.94 | 0 | 0 | 8.27 | 1 | 2.55 |
| 284613 | EV3.U.dXU0UXldE16o | CYB561D1 | 9.32 | −4.78 | 0 | 0 | −0.48 | 0.38 | 0.39 |
| | 30kJd76O10lXs7zoio | | 9.05 | −5.88 | 0 | 0 | 1.28 | 0.78 | 0.39 |
| 29883 | igaMoUQTSko3j0_dEo | CNOT7 | 8.13 | 11.03 | 0 | 0 | 7.45 | 1 | 2.55 |
| 26986 | Z3lXXQiQCQO_rgae.U | PABPC1 | 8.21 | 5.36 | 0 | 0 | 0.46 | 0.61 | 2.55 |
| 7295 | lTa18SWdtIdSSUI64I | TXN | 7.87 | 10.36 | 0 | 0 | 6.8 | 1 | 2.55 |
| 51014 | rrSTqTOwDtf96_Xdzk | TMED7 | 8.22 | 5.8 | 0 | 0 | 1.16 | 0.76 | 2.55 |
| 83543 | cro4LO7ubuTqLuboeo | C9orf58 | 9.81 | −9.73 | 0 | 0 | 6.15 | 1 | 0.39 |
| | NYhQiQ484OkLpCAaro | | 8.77 | −6.48 | 0 | 0 | 2.16 | 0.9 | 0.39 |
| 81603 | fiqeVovq1_Xo.4RJB8 | TRIM8 | 7.86 | 4.49 | 0 | 0.01 | −0.96 | 0.28 | 2.54 |
| 3892 | uRZZVcW6TdecWaXRao | KRT86 | 9.18 | −4.73 | 0 | 0 | −0.57 | 0.36 | 0.39 |
| | NuI56tTXBLiz1BI1qo | | 9.06 | −5.94 | 0 | 0 | 1.37 | 0.8 | 0.39 |
| 647319 | fF0qErN7u28RAEvcqo | VEZF1L1 | 9.13 | −5.57 | 0 | 0 | 0.8 | 0.69 | 0.39 |
| | EXgQxRRVTOVcQQAIqo | | 8.89 | −5.59 | 0 | 0 | 0.83 | 0.7 | 0.39 |
| | ESCov8IoD60ebtN2Ko | | 9.01 | −4.83 | 0 | 0 | −0.4 | 0.4 | 0.39 |
| | flNOuHtOvcSA5XU7tU | | 8.02 | 4.25 | 0 | 0.01 | −1.39 | 0.2 | 2.54 |
| | TikHp8jRoNUCKRKH6o | | 9.11 | −5 | 0 | 0 | −0.12 | 0.47 | 0.39 |
| 6597 | fW1HXXHs.z6ErSHZao | SMARCA4 | 9.25 | −5.32 | 0 | 0 | 0.4 | 0.6 | 0.39 |
| | f3o7s_J67V76qCio | | 9.05 | −4.9 | 0 | 0 | −0.28 | 0.43 | 0.4 |
| 26985 | BSnouS6ZDfJ3cSbY30 | AP3M1 | 10.23 | 2.65 | 0.02 | 0.06 | −4.21 | 0.01 | 2.53 |
| 644316 | uIjqv_dLQUlSnouS64 | FLJ43315 | 9.62 | 2.88 | 0.01 | 0.04 | −3.81 | 0.02 | 2.53 |
| 26034 | Zufk46g7h.yR5Ou.qo | PIP3-E | 8.99 | −5.94 | 0 | 0 | 1.37 | 0.8 | 0.4 |
| | KUM97_MxFKKBOKKH_o | | 9.14 | −4.61 | 0 | 0 | −0.76 | 0.32 | 0.4 |
| | lz3dA9fim4lFmVJe10 | | 8.72 | 2.7 | 0.02 | 0.05 | −4.13 | 0.02 | 2.52 |
| 6662 | ruKinF6Ko01R4SF8N8 | SOX9 | 8.12 | 5.76 | 0 | 0 | 1.09 | 0.75 | 2.52 |
| 64081 | QovYhSXqQRJiB_3c8A | PBLD | 9.01 | 2.6 | 0.02 | 0.06 | −4.3 | 0.01 | 2.52 |
| 50808 | WunPH_9_tfRKl51NUU | AK3 | 7.89 | 9.85 | 0 | 0 | 6.28 | 1 | 2.52 |
| 80829 | rl77DuShX3X9OoiErI | ZFP91 | 7.92 | 4.52 | 0 | 0.01 | −0.92 | 0.29 | 2.51 |
| 8915 | Hl3.4x6KBH46LuJRcI | BCL10 | 7.81 | 11.21 | 0 | 0 | 7.61 | 1 | 2.51 |
| 79752 | lTulCXJNOiUgLMl_e0 | ZFAND1 | 8.27 | 12.14 | 0 | 0 | 8.44 | 1 | 2.51 |
| 51762 | lgiE9f.X7xNQqqRKro | RAB8B | 9.13 | −5.73 | 0 | 0 | 1.05 | 0.74 | 0.4 |
| 54407 | BvIpQQ9yzp_kCLnEU | SLC38A2 | 8.4 | 5.02 | 0 | 0 | −0.08 | 0.48 | 2.51 |
| | 06lnSCCXUd1JBLt9Sg | | 8.45 | −6.19 | 0 | 0 | 1.74 | 0.85 | 0.4 |
| 221035 | f90lDU9EJ_k_E7nnL8 | REEP3 | 7.54 | 11.32 | 0 | 0 | 7.71 | 1 | 2.51 |
| 10534 | 0jAjDVneDlSnld1QnY | SSSCA1 | 8.2 | −8.59 | 0 | 0 | 4.89 | 0.99 | 0.4 |
| | ZEF7Ln6t4faSV2rEt4 | | 8.16 | −8.63 | 0 | 0 | 4.94 | 0.99 | 0.4 |
| 4218 | 6nhZEkt6fj.SW00_r0 | RAB8A | 7.57 | 12.83 | 0 | 0 | 9.01 | 1 | 2.51 |
| 7555 | fqCL4tIUsJW16vX4E4 | CNBP | 7.96 | 5.02 | 0 | 0 | −0.07 | 0.48 | 2.5 |
| 83875 | uMBHih1AKqkKKCKpKo | BCDO2 | 8.95 | −5.51 | 0 | 0 | 0.71 | 0.67 | 0.4 |
| | Qn52erfo7avYUfpY6g | | 8.13 | 2.66 | 0.02 | 0.06 | −4.21 | 0.01 | 2.5 |
| 56616 | Z4.LH71d76jlL7pKqI | DIABLO | 9.01 | −5.71 | 0 | 0 | 1.02 | 0.73 | 0.4 |
| 4149 | rnkulnV6lsoDiYwY4Q | MAX | 7.74 | 8.34 | 0 | 0 | 4.6 | 0.99 | 2.5 |
| 10914 | KXojSHvn9k47Oy7dOE | PAPOLA | 8.18 | 3.31 | 0.01 | 0.02 | −3.05 | 0.05 | 2.5 |
| 54915 | Nov4vgk4A65U5eGdSY | YTHDF1 | 8.28 | 2.86 | 0.02 | 0.04 | −3.84 | 0.02 | 2.49 |
| 143279 | 0Piynigiiq_t_e3Suk | HECTD2 | 7.94 | 5.47 | 0 | 0 | 0.64 | 0.66 | 2.49 |
| 6235 | BmAPaUq92d_27e9AWk | RPS29 | 8.01 | 8.61 | 0 | 0 | 4.92 | 0.99 | 2.49 |
| 9538 | iRwF4H.Qdb666ikmpI | EI24 | 7.76 | 5.08 | 0 | 0 | 0.01 | 0.5 | 2.49 |
| 284930 | rOA0CAAwOIOUgE6ouo | LOC284930 | 8.95 | −5.61 | 0 | 0 | 0.86 | 0.7 | 0.4 |
| | BUJ07kCI3kHSBJ0Qqo | | 8.99 | −5.74 | 0 | 0 | 1.05 | 0.74 | 0.4 |
| 26130 | 6DE0YpSe7j94hcjiLU | GAPVD1 | 7.7 | 9.48 | 0 | 0 | 5.89 | 1 | 2.48 |
| 8886 | lofUF_Hnidenyffq9c | DDX18 | 7.59 | 15.39 | 0 | 0 | 10.88 | 1 | 2.48 |
| 6146 | HQjRbhNYrl.dQCs.gM | RPL22 | 8.39 | 3.11 | 0.01 | 0.03 | −3.4 | 0.03 | 2.48 |
| | WaSZeoQrfSBxySMP6o | | 9.02 | −5.44 | 0 | 0 | 0.6 | 0.65 | 0.4 |
| 3727 | 3nGLUT17_w1_vZWv94 | JUND | 10.42 | 2.88 | 0.01 | 0.04 | −3.82 | 0.02 | 2.48 |
| 8470 | W5dWOuc9PtRXFIOHmo | SORBS2 | 9.12 | −4.39 | 0 | 0.01 | −1.13 | 0.24 | 0.4 |
| | xgoK4ArK4o7qooqKCo | | 9.21 | −5.32 | 0 | 0 | 0.41 | 0.6 | 0.4 |
| 3638 | QUUtJIOgnyKB_XuJno | INSIG1 | 7.86 | 9.07 | 0 | 0 | 5.44 | 1 | 2.48 |
| 6152 | 9CHkOnnnCkXECkkXCQ | RPL24 | 7.6 | 11.99 | 0 | 0 | 8.31 | 1 | 2.48 |
| 29080 | lN55c8r33uE7l1SS4E | CCDC59 | 7.63 | 9.27 | 0 | 0 | 5.67 | 1 | 2.48 |
| 2171 | 0C.ggFEnjpIAHSHt5A | FABP5 | 7.34 | 14.27 | 0 | 0 | 10.11 | 1 | 2.48 |
| 7178 | ihNxCNaiNmhq_5eiug | TPT1 | 8.11 | 5.17 | 0 | 0 | 0.17 | 0.54 | 2.48 |
| 6698 | xHict9_dq5P4o6P6o | SPRR1A | 9.05 | −4.39 | 0 | 0.01 | −1.14 | 0.24 | 0.4 |
| 158293 | 6kq6kuInnOg0OhAeEo | FAM120AOS | 7.72 | 6.53 | 0 | 0 | 2.23 | 0.9 | 2.47 |
| 284058 | HnVfl7oE_3rXJ7r1T4 | KIAA1267 | 7.56 | 11.76 | 0 | 0 | 8.11 | 1 | 2.47 |
| 23603 | xWypO69AiCSipCoC8U | CORO1C | 7.83 | 3.65 | 0 | 0.02 | −2.43 | 0.08 | 2.47 |
| | TjqA8uui7tOBTtl7HY | | 7.99 | 10.77 | 0 | 0 | 7.2 | 1 | 2.47 |
| | 6AnlNC0SlKtN0KdF6o | | 8.87 | −6.08 | 0 | 0 | 1.58 | 0.83 | 0.4 |
| 9698 | rtyX5WJ.XxDSJV3Rfs | PUM1 | 8.36 | 6.15 | 0 | 0 | 1.67 | 0.84 | 2.47 |
| 51031 | ri7UigEgKDi7uG_eRk | GLOD4 | 7.9 | 7.64 | 0 | 0 | 3.74 | 0.98 | 2.47 |
| 2152 | r6m4FFOVJYAn.iqeH0 | F3 | 7.73 | 5.34 | 0 | 0 | 0.44 | 0.61 | 2.47 |
| 79698 | oIiGCVRiURXHcQigKo | ZMAT4 | 9.05 | −5.79 | 0 | 0 | 1.14 | 0.76 | 0.41 |
| | H1aD_l3qEQV96gT9qo | | 8.94 | −6.44 | 0 | 0 | 2.11 | 0.89 | 0.41 |
| 10109 | ZigmnpB4KegR_cejDY | ARPC2 | 7.99 | 5.51 | 0 | 0 | 0.71 | 0.67 | 2.46 |
| 7321 | Kksnsgs7CDO46uy08k | UBE2D1 | 7.61 | 14.79 | 0 | 0 | 10.48 | 1 | 2.46 |
| 2782 | H9bUEHeyJ3eRXxV.UU | GNB1 | 8.21 | 2.79 | 0.02 | 0.05 | −3.98 | 0.02 | 2.46 |
| 5049 | TvooBF4ogEBRT5eHp0 | PAFAH1B2 | 7.59 | 13.29 | 0 | 0 | 9.38 | 1 | 2.46 |
| 440359 | 3bZUb3XBnX0QjpAilE | LOC440359 | 8.35 | 6.52 | 0 | 0 | 2.22 | 0.9 | 2.45 |
| | c8ohusrR3sTvfXSQqo | | 9.02 | −5.89 | 0 | 0 | 1.29 | 0.78 | 0.41 |
| 220213 | rpMDt6JQX6S8ySiBHs | OTUD1 | 8.13 | 6.46 | 0 | 0 | 2.13 | 0.89 | 2.45 |
| 5836 | oNQKXoEQ6x0AEyeXao | PYGL | 9.2 | −5.66 | 0 | 0 | 0.95 | 0.72 | 0.41 |
| | o5yiuA3vkCeD8wryqo | | 9.02 | −5.73 | 0 | 0 | 1.04 | 0.74 | 0.41 |
| 140901 | HAT_7qEhibior.CUpE | STK35 | 8.05 | 4.14 | 0 | 0.01 | −1.58 | 0.17 | 2.44 |
| 474338 | fdeh7h.S6iOgu.SIHg | SUMO1P3 | 7.67 | 15.61 | 0 | 0 | 11.03 | 1 | 2.44 |
| 23399 | HmJX6jlt45XtQ7ih5c | DULLARD | 7.51 | 5.51 | 0 | 0 | 0.71 | 0.67 | 2.44 |
| 57179 | KSpegZe5qitHQ9AP94 | KIAA1191 | 7.95 | 4.39 | 0 | 0.01 | −1.14 | 0.24 | 2.44 |
| 125476 | 6mvSRT4Iv9cT2inld4 | C18orf37 | 7.61 | 16.36 | 0 | 0 | 11.5 | 1 | 2.44 |
| | TpCRGBESEUjERgEiqo | | 8.82 | −5.92 | 0 | 0 | 1.34 | 0.79 | 0.41 |
| | H0gbXSBZKJQigN1bKo | | 8.83 | −6.45 | 0 | 0 | 2.11 | 0.89 | 0.41 |
| 4809 | H3iJX1Xu15RTurSOx0 | NHP2L1 | 7.99 | 5.38 | 0 | 0 | 0.5 | 0.62 | 2.44 |
| 5634 | HeDqroXpPzelABKJSI | PRPS2 | 7.66 | 13.51 | 0 | 0 | 9.54 | 1 | 2.44 |
| | Et1LjHiy3T0inVwuqo | | 8.71 | −6.89 | 0 | 0 | 2.73 | 0.94 | 0.41 |
| 64100 | T9jiASS6A97gGDizqo | ELSPBP1 | 8.94 | −5.68 | 0 | 0 | 0.97 | 0.72 | 0.41 |
| | rOkKjJLcU.F8qce79c | | 8.17 | −6.8 | 0 | 0 | 2.61 | 0.93 | 0.41 |
| 3066 | QKBQNIEnSQVeD_Et0U | HDAC2 | 7.82 | 11.53 | 0 | 0 | 7.9 | 1 | 2.43 |
| 57187 | ElFofBQiGwIog6BDqo | THOC2 | 8.91 | −6.37 | 0 | 0 | 2.01 | 0.88 | 0.41 |
| 5501 | lGLhY85.f6XE9.Pea4 | PPP1CC | 8.16 | 3.32 | 0.01 | 0.02 | −3.03 | 0.05 | 2.43 |
| 10890 | BKn_Vf97C3fqNe7IJ4 | RAB10 | 8.11 | 5.59 | 0 | 0 | 0.82 | 0.7 | 2.43 |
| 55432 | ZS.1LRPrlPSh1J78Sg | YOD1 | 7.75 | 6.26 | 0 | 0 | 1.84 | 0.86 | 2.43 |
| 26154 | x5Qywnkq6BCA0fneqo | ABCA12 | 8.7 | −5.76 | 0 | 0 | 1.09 | 0.75 | 0.41 |
| | 6tRLgtR1K83kyslSkU | | 7.99 | 7.57 | 0 | 0 | 3.65 | 0.97 | 2.43 |
| 29058 | iedOPUKNJejlyKHI0U | C20orf30 | 8.19 | 3.78 | 0 | 0.01 | −2.21 | 0.1 | 2.42 |
| | QXqIDgCRrTxJ0v6c6o | | 9.02 | −4.95 | 0 | 0 | −0.2 | 0.45 | 0.41 |
| 4893 | Hr.Uil7.qn9UogI4B4 | NRAS | 8.34 | 3.5 | 0 | 0.02 | −2.7 | 0.06 | 2.42 |
| 11034 | x0pE0_gxKCyV5F5S4k | DSTN | 7.66 | 9.04 | 0 | 0 | 5.41 | 1 | 2.42 |
| | oJIiyDrryIp6i_BoOo | | 8.95 | −5.21 | 0 | 0 | 0.22 | 0.56 | 0.41 |
| 133383 | TlR9ju_9_2R8qE0NCg | C5orf35 | 7.78 | 15.24 | 0 | 0 | 10.78 | 1 | 2.41 |
| | uyIInOFPrK7U_dVyuo | | 8.93 | −5.1 | 0 | 0 | 0.04 | 0.51 | 0.41 |
| 57122 | xnSItd3DnXIUqH4VPI | NUP107 | 7.93 | 8.2 | 0 | 0 | 4.43 | 0.99 | 2.41 |
| 1486 | TapPpO6DkB7fhx3ojk | CTBS | 7.44 | 15.93 | 0 | 0 | 11.23 | 1 | 2.41 |
| | rhNHa3uH2uQ3X0qCWo | | 8.66 | −6 | 0 | 0 | 1.45 | 0.81 | 0.42 |
| 201895 | EujpL.ey.6oe6yd_j4 | C4orf34 | 8.13 | 8.47 | 0 | 0 | 4.75 | 0.99 | 2.41 |
| 10381 | l11UXKUbuJ517d7fPk | TUBB3 | 7.57 | 7.92 | 0 | 0 | 4.09 | 0.98 | 2.41 |
| 6120 | c_d7RUp4LkukS0qVPk | RPE | 9.63 | 2.78 | 0.02 | 0.05 | −4 | 0.02 | 2.41 |
| 3146 | x5P787D9KKDHgTeLXo | HMGB1 | 8.56 | 2.96 | 0.01 | 0.04 | −3.68 | 0.02 | 2.41 |
| 11177 | cX4LnsUuenkrPC1C.M | BAZ1A | 7.95 | 5.43 | 0 | 0 | 0.57 | 0.64 | 2.4 |
| 51026 | iq.jDdUtfLAj4iKeiQ | GOLT1B | 7.65 | 13.56 | 0 | 0 | 9.59 | 1 | 2.4 |
| | ECUSFSKp0fi_4ogCqo | | 8.91 | −4.86 | 0 | 0 | −0.34 | 0.42 | 0.42 |
| 23016 | N67a4UkKi_4bk4oC6o | EXOSC7 | 9.13 | −5.02 | 0 | 0 | −0.07 | 0.48 | 0.42 |
| 57045 | NRyo6oGDlA0h1FyJFI | TWSG1 | 7.63 | 10.58 | 0 | 0 | 7.02 | 1 | 2.4 |
| 199870 | iqSAkK_1K_x6Efd1UU | FAM76A | 7.6 | 13.52 | 0 | 0 | 9.55 | 1 | 2.4 |
| 1969 | ci7XlTlUtpVN3TX.ow | EPHA2 | 8.15 | 2.58 | 0.03 | 0.06 | −4.34 | 0.01 | 2.4 |
| 7326 | Tc56SFcOiHRecO_OeY | UBE2G1 | 7.45 | 12.11 | 0 | 0 | 8.41 | 1 | 2.4 |
| 1974 | KoV75wlUkJDXKyr8NU | EIF4A2 | 9.17 | 2.71 | 0.02 | 0.05 | −4.12 | 0.02 | 2.4 |
| 10124 | 6tUyy9I3nyO4Sk_Cns | ARL4A | 7.68 | 8.98 | 0 | 0 | 5.34 | 1 | 2.4 |
| 128239 | ES15d5RLd3vtVE3FQc | IQGAP3 | 8.07 | 3.69 | 0 | 0.01 | −2.36 | 0.09 | 2.39 |
| 29097 | rkS_KJSIffA018gW4U | CNIH4 | 7.78 | 4.6 | 0 | 0 | −0.79 | 0.31 | 2.39 |
| | HY1m.dEzTJj1jGqlKo | | 8.93 | −5.45 | 0 | 0 | 0.61 | 0.65 | 0.42 |
| 2958 | rkkenSKtKBItD9Kp3c | GTF2A2 | 8.03 | 10.99 | 0 | 0 | 7.41 | 1 | 2.39 |
| 54443 | f0gC47oTKKQ7uIfqr0 | ANLN | 7.85 | 7.33 | 0 | 0 | 3.33 | 0.97 | 2.39 |
| 10923 | 3Tp_7gB4krv78VMu94 | SUB1 | 7.97 | 6.4 | 0 | 0 | 2.04 | 0.89 | 2.39 |
| 56984 | ljHQFHyY4VRLN5dGug | PSMG2 | 8.12 | 8.13 | 0 | 0 | 4.34 | 0.99 | 2.39 |
| | f5Kb6DqHXqwOjouG6o | | 8.96 | −4.57 | 0 | 0 | −0.83 | 0.3 | 0.42 |
| | TeewU1IBpPjvn0b544 | | 7.51 | 14.57 | 0 | 0 | 10.32 | 1 | 2.39 |
| 7443 | laDrQERISKKUxIVvQg | VRK1 | 7.71 | 13.16 | 0 | 0 | 9.27 | 1 | 2.39 |
| 4802 | 9oEnnFVCH5RvveV0jo | NFYC | 8.33 | 6.81 | 0 | 0 | 2.63 | 0.93 | 2.38 |
| | fdyXDoVRATNzB.UVJI | | 8.25 | −7.11 | 0 | 0 | 3.04 | 0.95 | 0.42 |
| 27292 | TctET1UxT9u89E9VsU | DIMT1L | 7.85 | 6.65 | 0 | 0 | 2.41 | 0.92 | 2.38 |
| 90324 | HI7XSGoK.iCrSOspqU | CCDC97 | 8.27 | −9 | 0 | 0 | 5.37 | 1 | 0.42 |
| 57688 | E65AL7Uu3fvV9HIgTk | ZSWIM6 | 8 | 6.83 | 0 | 0 | 2.65 | 0.93 | 2.38 |
| | 9LPtSjecp3NR5KVe6o | | 8.91 | −5.89 | 0 | 0 | 1.29 | 0.78 | 0.42 |
| 64837 | fDRVT1ua6zdUp3E92o | KLC2 | 8.9 | −4.34 | 0 | 0.01 | −1.23 | 0.23 | 0.42 |
| 64849 | WCCu6Jmaaeeuee9GWc | SLC13A3 | 8.03 | 4.39 | 0 | 0.01 | −1.14 | 0.24 | 2.38 |
| 147184 | od3Qq3Rvkk98UilPqU | TMEM99 | 7.58 | 14.24 | 0 | 0 | 10.09 | 1 | 2.37 |
| 140890 | 9s.Lqg6Ai_V_QsOCU | SFRS12 | 8.18 | 4.29 | 0 | 0.01 | −1.31 | 0.21 | 2.37 |
| 118460 | 97u.3mlejDnsk9Ktj4 | EXOSC6 | 7.75 | 4.67 | 0 | 0 | −0.66 | 0.34 | 2.37 |
| 3344 | THlLI4UtELgUfdL5Q0 | FOXN2 | 8.03 | 5.48 | 0 | 0 | 0.66 | 0.66 | 2.36 |
| | iHewoL1QMQHzBVJ7rc | | 7.87 | 8.21 | 0 | 0 | 4.44 | 0.99 | 2.36 |
| 403244 | fX16nuS9A33F4V4_io | OR2T35 | 8.94 | −4.9 | 0 | 0 | −0.27 | 0.43 | 0.42 |
| 8570 | fXl3eKMCQ9P91RXaV0 | KHSRP | 7.73 | 8.83 | 0 | 0 | 5.17 | 0.99 | 2.36 |
| 389898 | 35XRwwT7h.ntC7ItVU | UBE2NL | 7.75 | 8.15 | 0 | 0 | 4.38 | 0.99 | 2.36 |
| 144455 | ovtEinu7lcR4Uq.sAU | E2F7 | 7.61 | 9.3 | 0 | 0 | 5.7 | 1 | 2.36 |
| | llGfH57t5ug93Xe1XU | | 7.52 | 7.63 | 0 | 0 | 3.72 | 0.98 | 2.36 |
| 390 | xgu_Ce51XwNukoiPCs | RND3 | 7.6 | 20.83 | 0 | 0 | 13.87 | 1 | 2.36 |
| 51582 | WrkH_LX6fhzEpfgfTo | AZIN1 | 7.76 | 6.82 | 0 | 0 | 2.64 | 0.93 | 2.35 |
| 8821 | TE5xJ46f1ULHEhdSKo | INPP4B | 9.07 | −4.62 | 0 | 0 | −0.75 | 0.32 | 0.42 |
| 3315 | H6qVIJ5ANY4h5ZQsCU | HSPB1 | 7.9 | 6.25 | 0 | 0 | 1.82 | 0.86 | 2.35 |
| 55515 | 0qiV3sT_wM.KgJauqo | ACCN4 | 8.69 | −5.86 | 0 | 0 | 1.24 | 0.78 | 0.43 |
| | Zdx1BAdRXxU5SQIeKo | | 8.99 | −5.42 | 0 | 0 | 0.56 | 0.64 | 0.43 |
| | TI4P7ZfPoZYpSJbuKo | | 8.83 | −5.36 | 0 | 0 | 0.46 | 0.61 | 0.43 |
| | WuJAw1XUXUR1YsJeKo | | 8.75 | −6.1 | 0 | 0 | 1.61 | 0.83 | 0.43 |
| 84262 | cNN7k4aoq6BK1aKLbg | PSMG3 | 7.96 | 2.55 | 0.03 | 0.06 | −4.39 | 0.01 | 2.35 |
| 27288 | 6z1NPjQRAwOu89qCKo | RBMXL2 | 9.02 | −5.14 | 0 | 0 | 0.12 | 0.53 | 0.43 |
| | fpS7owpLCv_LeKI6eo | | 9.19 | −4.66 | 0 | 0 | −0.69 | 0.33 | 0.43 |
| 23258 | QTlqO7TblAxRP176RI | RAB6IP1 | 7.69 | 7.75 | 0 | 0 | 3.87 | 0.98 | 2.34 |
| 9519 | iZLD3TtVMJIntEu5HE | TBPL1 | 7.86 | 9.13 | 0 | 0 | 5.51 | 1 | 2.34 |
| | WPjkQLCegA3irp8uok | | 7.58 | 10.15 | 0 | 0 | 6.59 | 1 | 2.34 |
| | TNEEISoKYAKlJRQdqo | | 8.76 | −5.36 | 0 | 0 | 0.47 | 0.62 | 0.43 |
| | Kpbj6CT4jLesVCgxao | | 8.98 | −4.81 | 0 | 0 | −0.42 | 0.4 | 0.43 |
| | cnf_nNV.UWUyqT06Go | | 8.68 | −6.03 | 0 | 0 | 1.51 | 0.82 | 0.43 |
| | BvhWoCe.nOy0msRkqo | | 8.95 | −5.74 | 0 | 0 | 1.07 | 0.74 | 0.43 |
| 146547 | T5LcfXnXnh3XjUieKo | PRSS36 | 9.17 | −5.19 | 0 | 0 | 0.19 | 0.55 | 0.43 |
| | fCkrIgODj6c4ZVX16o | | 8.95 | −5.47 | 0 | 0 | 0.64 | 0.66 | 0.43 |
| 10927 | KU.1V0Vwd3z3llEOQk | SPIN1 | 7.72 | 8.44 | 0 | 0 | 4.72 | 0.99 | 2.34 |
| 91368 | 0hCt0d7ZUpIJECuSz4 | CDKN2AIPNL | 7.59 | 6.87 | 0 | 0 | 2.72 | 0.94 | 2.33 |
| | QgJfl0rVYYNeV8N7qo | | 8.77 | −6.36 | 0 | 0 | 1.99 | 0.88 | 0.43 |
| | QkgydO_aO3.qMJAe6o | | 8.57 | −6.73 | 0 | 0 | 2.51 | 0.92 | 0.43 |
| | Nfk_nIOO_jpRVSC06o | | 8.96 | −5.3 | 0 | 0 | 0.37 | 0.59 | 0.43 |
| 6156 | Z_qltWdcKSgjrpZAgg | RPL30 | 7.86 | 5.63 | 0 | 0 | 0.9 | 0.71 | 2.33 |
| 861 | crSKWNZIFIG.1XXoe8 | RUNX1 | 7.52 | 16.88 | 0 | 0 | 11.81 | 1 | 2.33 |
| | l1wUQ7uRdNMoKBEDqo | | 8.77 | −5.31 | 0 | 0 | 0.39 | 0.6 | 0.43 |
| 9412 | oSgRSbyewC_SQ.Ppy0 | MED21 | 7.51 | 7.44 | 0 | 0 | 3.47 | 0.97 | 2.32 |
| | NHSl9HIg9QrtMSc_io | | 8.9 | −5.36 | 0 | 0 | 0.47 | 0.62 | 0.43 |
| | TNIhRUhEIdXknueqyQ | | 7.81 | 9.48 | 0 | 0 | 5.89 | 1 | 2.32 |
| | EXknuzddO6XQCTF76o | | 9.08 | −5.7 | 0 | 0 | 1.01 | 0.73 | 0.43 |
| | uycLILHlK8.KgP8BQQ | | 7.75 | 10.2 | 0 | 0 | 6.64 | 1 | 2.32 |
| 5573 | cuQcHh3vPjV915X9Uo | PRKAR1A | 8.62 | 5.43 | 0 | 0 | 0.58 | 0.64 | 2.32 |
| 26060 | x4IHYfzuNOs_sxO6ro | APPL1 | 9 | −5.19 | 0 | 0 | 0.19 | 0.55 | 0.43 |
| 54585 | Wrr_JfjyH.jucJSEpI | LZTFL1 | 7.88 | 5.59 | 0 | 0 | 0.83 | 0.7 | 2.32 |
| 8161 | ESkXp4u56LijfgSAfU | COIL | 8.12 | 4.08 | 0 | 0.01 | −1.67 | 0.16 | 2.32 |
| 136051 | Z6ijZeJUqK0KeS4kOM | ZNF786 | 8.41 | 2.86 | 0.02 | 0.04 | −3.86 | 0.02 | 2.32 |
| 169522 | rSQngp84sxRTGSoiqI | KCNV2 | 8.64 | −5.04 | 0 | 0 | −0.05 | 0.49 | 0.43 |
| 113828 | 6VRlHVRUIj66DAKgio | FAM83F | 8.96 | −5.57 | 0 | 0 | 0.8 | 0.69 | 0.43 |
| 2030 | oHjV5_5KUuinfqfogQ | SLC29A1 | 7.76 | 4.88 | 0 | 0 | −0.31 | 0.42 | 2.32 |
| 7048 | rplyA9R_RbKk54xTVA | TGFBR2 | 7.88 | 4.48 | 0 | 0.01 | −0.99 | 0.27 | 2.32 |
| 388962 | Zo_aM.F3tVAZ4UZXp4 | BOLA3 | 8.18 | 6.43 | 0 | 0 | 2.08 | 0.89 | 2.32 |
| 6418 | xU7g3q6jDodJ3t50OU | SET | 7.81 | 6.05 | 0 | 0 | 1.53 | 0.82 | 2.32 |
| 9403 | fl07nu53_AoOEkhxAk | 15-Sep | 7.93 | 8.05 | 0 | 0 | 4.25 | 0.99 | 2.31 |
| 64778 | xV6RCA9S07gkE5X_10 | FNDC3B | 7.68 | 12.59 | 0 | 0 | 8.81 | 1 | 2.31 |
| | KOh3bXtFSnouSaZDdo | | 9.79 | 2.41 | 0.03 | 0.08 | −4.63 | 0.01 | 2.31 |
| 246184 | ZS3qC7LUvp8ksv2_qo | CDC26 | 8.99 | −5.48 | 0 | 0 | 0.65 | 0.66 | 0.43 |
| 23468 | ZijmgBAiAgEkCJwKKo | CBX5 | 8.8 | −5.48 | 0 | 0 | 0.66 | 0.66 | 0.43 |
| | 0N6VfLhKKS.5VIKVYc | | 8.15 | −7.06 | 0 | 0 | 2.97 | 0.95 | 0.43 |
| 653573 | Bk9InECgygLKsu_j3o | GCUD2 | 7.96 | 5.3 | 0 | 0 | 0.36 | 0.59 | 2.31 |
| 5991 | Bjr0kenz.vM_Dkopqo | RFX3 | 8.85 | −6.09 | 0 | 0 | 1.59 | 0.83 | 0.43 |
| 51125 | ZKNqnnzl.b81_q.iJk | GOLGA7 | 7.78 | 6.81 | 0 | 0 | 2.63 | 0.93 | 2.31 |
| 6161 | 9t9J9_lB8v5RIj73.k | RPL32 | 8.2 | −8.01 | 0 | 0 | 4.2 | 0.99 | 0.43 |
| 25790 | cJ4mQJwTXlJxd7geKo | CCDC19 | 9.07 | −4.72 | 0 | 0 | −0.57 | 0.36 | 0.43 |
| 4144 | i65p6U6ICeH6eu6xIg | MAT2A | 8.1 | 4.72 | 0 | 0 | −0.58 | 0.36 | 2.31 |
| 23023 | Kgo4n_6QkA0kh5eEqo | TMCC1 | 8.99 | −5 | 0 | 0 | −0.11 | 0.47 | 0.43 |
| 51123 | 0TVTvweER6qdL7uew4 | ZNF706 | 7.78 | 6.59 | 0 | 0 | 2.32 | 0.91 | 2.3 |
| 92259 | 96pV17lCrGzPuCwJdE | MRPS36 | 7.59 | 6.68 | 0 | 0 | 2.44 | 0.92 | 2.3 |
| 10269 | HqNB7GX_s3hTAt.51k | ZMPSTE24 | 7.73 | 9.64 | 0 | 0 | 6.06 | 1 | 2.3 |
| | rueRfRt4ukvsM4KoIo | | 8.6 | −5.67 | 0 | 0 | 0.95 | 0.72 | 0.43 |
| 57590 | ijOuV7s1SI5yAHvf50 | WDFY1 | 7.96 | 3.17 | 0.01 | 0.03 | −3.29 | 0.04 | 2.3 |
| | Zrot0pPh9UrgoKuP_o | | 8.9 | −4.13 | 0 | 0.01 | −1.59 | 0.17 | 0.43 |
| 8771 | fIkI3bX18hIpSSoe6o | TNFRSF6B | 8.75 | −5.92 | 0 | 0 | 1.34 | 0.79 | 0.43 |
| 811 | NV1BeqpLruiCUSl4j8 | CALR | 7.63 | 6.11 | 0 | 0 | 1.62 | 0.83 | 2.3 |
| | KQemKt_559N0yoC6Co | | 8.78 | −5 | 0 | 0 | −0.11 | 0.47 | 0.44 |
| 91408 | o10FHdcd_l_BcXer6M | BTF3L4 | 7.73 | 18.21 | 0 | 0 | 12.57 | 1 | 2.3 |
| 4154 | lNSX0dSevADvkfNJBU | MBNL1 | 7.99 | 6.14 | 0 | 0 | 1.66 | 0.84 | 2.29 |
| 389641 | QRT9ETXiXU_.4tJ7b0 | LOC389641 | 7.62 | 16.1 | 0 | 0 | 11.33 | 1 | 2.29 |
| 10945 | 0.cj6oyogKilSgrdV4 | KDELR1 | 7.59 | 5.73 | 0 | 0 | 1.05 | 0.74 | 2.29 |
| 9364 | NnjnUboP3fkB5MIsdU | RAB28 | 7.89 | 5.42 | 0 | 0 | 0.56 | 0.64 | 2.29 |
| 57727 | xIKKv3p7r3A6JVKCeU | NCOA5 | 7.53 | 8.89 | 0 | 0 | 5.24 | 0.99 | 2.29 |
| 27257 | NroEkhQnoJIgvgLkpU | LSM1 | 7.47 | 12.54 | 0 | 0 | 8.77 | 1 | 2.29 |
| | HCgEhEXkS_gOiDgjEM | | 8 | 10.36 | 0 | 0 | 6.81 | 1 | 2.29 |
| 2026 | T5vrUiaKe7l6qL.Xcw | ENO2 | 7.81 | 4.96 | 0 | 0 | −0.18 | 0.46 | 2.29 |
| 9662 | clf.Luzyjup6.n.cUU | CEP135 | 8.1 | 3.69 | 0 | 0.01 | −2.36 | 0.09 | 2.29 |
| | rteiuy6IkugORQojio | | 8.85 | −4.4 | 0 | 0.01 | −1.12 | 0.25 | 0.44 |
| | TYhQiS484OkLpCAaro | | 8.51 | −5.59 | 0 | 0 | 0.84 | 0.7 | 0.44 |
| 627 | Efnut3_6SC79OTpJKU | BDNF | 7.75 | 10.23 | 0 | 0 | 6.67 | 1 | 2.29 |
| 92703 | 6k.AKLpXv97vAFU.rk | TMEM183A | 8.06 | 3.93 | 0 | 0.01 | −1.94 | 0.13 | 2.29 |
| | cnKTyrAwxMI2nqbrp0 | | 7.89 | 4.11 | 0 | 0.01 | −1.62 | 0.17 | 2.29 |
| 38 | 06jt6JSj760.h05fgk | ACAT1 | 7.55 | 10.87 | 0 | 0 | 7.3 | 1 | 2.29 |
| | 39I0p13cP6OOtDkKKo | | 8.74 | −4.84 | 0 | 0 | −0.38 | 0.41 | 0.44 |
| 3491 | cLA6ipPU1fXgqoR1OI | CYR61 | 8.23 | 3.88 | 0 | 0.01 | −2.04 | 0.12 | 2.29 |
| | xJUiIBlXaAVZ3V3JIA | | 8.22 | −7.78 | 0 | 0 | 3.92 | 0.98 | 0.44 |
| 9474 | xEhAFLtKX_Syn4uB94 | ATG5 | 7.96 | 9.17 | 0 | 0 | 5.55 | 1 | 2.28 |
| 8334 | 33obrCQopAnZlAmA1Y | HIST1H2AC | 7.73 | 5.2 | 0 | 0 | 0.2 | 0.55 | 2.28 |
| 51655 | ZtKFRqk837e49avnuE | RASD1 | 7.62 | 7.49 | 0 | 0 | 3.54 | 0.97 | 2.28 |
| 3336 | Nkixi3imiosOkoQm.I | HSPE1 | 7.85 | 5.89 | 0 | 0 | 1.3 | 0.79 | 2.28 |
| 28969 | EA6JXik0nPMfqHcXdE | BZW2 | 7.86 | 10.16 | 0 | 0 | 6.61 | 1 | 2.28 |
| 158160 | rwyeruuk5k7LVBx0oo | HSD17B7P2 | 8.21 | 3.1 | 0.01 | 0.03 | −3.42 | 0.03 | 2.28 |
| | QTx_4Pn7NQDkhEOeqo | | 8.81 | −7.06 | 0 | 0 | 2.97 | 0.95 | 0.44 |
| | 3T9SoLQjRACquPeuqo | | 8.61 | −5.38 | 0 | 0 | 0.5 | 0.62 | 0.44 |
| 1958 | Ebfrl.7uOZfnjp_E7k | EGR1 | 8.37 | 2.95 | 0.01 | 0.04 | −3.69 | 0.02 | 2.28 |
| 9480 | oXuDF0n0HqXUxLS_uo | ONECUT2 | 8.79 | −5.15 | 0 | 0 | 0.13 | 0.53 | 0.44 |
| | WlG0AfeJSmAyAiq1yo | | 8.86 | −4.47 | 0 | 0.01 | −1 | 0.27 | 0.44 |
| | WVCDnpVQ3UAvyuC9ao | | 9.05 | −5.37 | 0 | 0 | 0.49 | 0.62 | 0.44 |
| 6908 | f_5HiBFmSbh7i_dMW4 | TBP | 7.74 | 7.66 | 0 | 0 | 3.76 | 0.98 | 2.28 |
| 91298 | BjSoBf6h94Sk3rgiEM | C12orf29 | 7.77 | 10.97 | 0 | 0 | 7.39 | 1 | 2.28 |
| 7594 | Q3CmaEfSE8RwNLnCxI | ZNF43 | 8.1 | −10.98 | 0 | 0 | 7.4 | 1 | 0.44 |
| | 0tnVvUrZokCtjRvdjQ | | 7.62 | 10.93 | 0 | 0 | 7.36 | 1 | 2.28 |
| 7324 | Newpugyi_dLo_vc77o | UBE2E1 | 8.12 | 3.2 | 0.01 | 0.03 | −3.25 | 0.04 | 2.28 |
| | NMdClq5rp0xE6JCgoU | | 7.82 | 4.6 | 0 | 0 | −0.78 | 0.31 | 2.27 |
| | T_XgCX1mqPwZP61Cqo | | 8.72 | −5.81 | 0 | 0 | 1.17 | 0.76 | 0.44 |
| 5480 | i4S65HUyJ1CQeOpOFs | PPIC | 7.7 | 9.09 | 0 | 0 | 5.46 | 1 | 2.27 |
| 51635 | N7pDE5QXqXrowUEI6o | DHRS7 | 8.87 | −4.33 | 0 | 0.01 | −1.24 | 0.23 | 0.44 |
| 123811 | KdWqh_v9tQo76S6EfI | C16orf63 | 7.64 | 10.5 | 0 | 0 | 6.94 | 1 | 2.27 |
| 3093 | xtPfn5H1XPhE4Ce764 | UBE2K | 7.71 | 5.53 | 0 | 0 | 0.73 | 0.68 | 2.27 |
| 143903 | EGF176FVG.6ezX81SU | LAYN | 7.53 | 10.06 | 0 | 0 | 6.5 | 1 | 2.27 |
| 57198 | ZoOoiqC36SoDV.6URI | ATP8B2 | 7.73 | 4.37 | 0 | 0.01 | −1.17 | 0.24 | 2.27 |
| 1399 | HtXEooEul_ffIa30e4 | CRKL | 7.97 | 8.09 | 0 | 0 | 4.29 | 0.99 | 2.27 |
| 83941 | Zfo6_okjY.xfoxXn_o | TM2D1 | 8.02 | 9.35 | 0 | 0 | 5.76 | 1 | 2.27 |
| 56942 | ZqSpvyx7dV.FJAXh9E | C16orf61 | 8.01 | 3.22 | 0.01 | 0.03 | −3.21 | 0.04 | 2.27 |
| 25862 | N1ycfqKeK6iD50JUos | USP49 | 8.4 | 2.82 | 0.02 | 0.04 | −3.92 | 0.02 | 2.27 |
| 10049 | EXn.T7t4DuJRsu2154 | DNAJB6 | 8.01 | 6.5 | 0 | 0 | 2.18 | 0.9 | 2.27 |
| 161394 | 0ulgB09G0HnQF1dI6o | C14orf174 | 8.87 | −6.06 | 0 | 0 | 1.54 | 0.82 | 0.44 |
| 5500 | xnlXiCIfDUJePscuk0 | PPP1CB | 7.75 | 9.41 | 0 | 0 | 5.81 | 1 | 2.26 |
| 64326 | WV1.OF.qE_oMVJQd1E | RFWD2 | 7.64 | 8 | 0 | 0 | 4.19 | 0.99 | 2.26 |
| 2920 | N244TNE7SUe4yKeKDU | CXCL2 | 7.4 | 13.35 | 0 | 0 | 9.42 | 1 | 2.26 |
| 27242 | rArpuh1f7urqv7qy64 | TNFRSF21 | 7.88 | 4.2 | 0 | 0.01 | −1.47 | 0.19 | 2.26 |
| 6208 | Q76eVKO6VIrkKJ6s0U | RPS14 | 7.67 | 4.42 | 0 | 0.01 | −1.09 | 0.25 | 2.26 |
| 9287 | 9UX786Sv30derc466o | TAAR2 | 8.9 | −4.36 | 0 | 0.01 | −1.19 | 0.23 | 0.44 |
| 4686 | ZeOr8VJxUskwf9Enao | NCBP1 | 9.41 | −4.5 | 0 | 0.01 | −0.95 | 0.28 | 0.44 |
| 6259 | H_Rcgy5zkSJq5_L77Y | RYK | 8.05 | 3.8 | 0 | 0.01 | −2.17 | 0.1 | 2.25 |
| 5366 | Nr2A51_0Ty7k1xAC40 | PMAIP1 | 7.75 | 11.9 | 0 | 0 | 8.24 | 1 | 2.25 |
| 201965 | E7Kr3rjrrF3zxfOwBE | RWDD4A | 8.85 | 4.49 | 0 | 0.01 | −0.96 | 0.28 | 2.25 |
| 10094 | HklFt1IlepJP9SQjsQ | ARPC3 | 7.72 | 4.41 | 0 | 0.01 | −1.1 | 0.25 | 2.25 |
| 84928 | TFuzS7yO5NW.7T1dIc | TMEM209 | 7.87 | 7.9 | 0 | 0 | 4.06 | 0.98 | 2.25 |
| 148534 | 197_QDfV4veBUkU0sU | TMEM56 | 7.68 | 6.51 | 0 | 0 | 2.2 | 0.9 | 2.25 |
| | 0h1OAuTzRgng.uwk_4 | | 7.81 | 5.13 | 0 | 0 | 0.09 | 0.52 | 2.24 |
| 6611 | ike2dzggSeqZez7Xug | SMS | 7.75 | 9.76 | 0 | 0 | 6.19 | 1 | 2.24 |
| 5725 | EgHXV_3JXu9nuhnsik | PTBP1 | 7.85 | 3.32 | 0.01 | 0.02 | −3.02 | 0.05 | 2.24 |
| 23760 | leyzT6ifKZE6A4iVpk | PITPNB | 7.94 | 4.35 | 0 | 0.01 | −1.21 | 0.23 | 2.24 |
| 23517 | cgpJMPb7OKZegg_fYQ | SKIV2L2 | 8.17 | −5.67 | 0 | 0 | 0.96 | 0.72 | 0.45 |
| 10627 | ZPwXFJX3VUMHutzEi0 | MRCL3 | 7.83 | 8.12 | 0 | 0 | 4.34 | 0.99 | 2.24 |
| 84988 | ix7EoR6Vd0rSCE5eio | PPP1R16A | 8.87 | −4.49 | 0 | 0.01 | −0.97 | 0.27 | 0.45 |
| 51588 | B.UV.5enXpF7F3od1w | PIAS4 | 7.63 | 5.71 | 0 | 0 | 1.02 | 0.73 | 2.24 |
| 8869 | reEHuCUV6nEgFEt9Uk | ST3GAL5 | 7.48 | 7.85 | 0 | 0 | 4 | 0.98 | 2.23 |
| 9404 | WRv9U.le6dHt8Q0ee4 | LPXN | 7.45 | 8.38 | 0 | 0 | 4.65 | 0.99 | 2.23 |
| 2764 | TlKkvVHj8jrUIw3T0o | GMFB | 8.61 | 3.01 | 0.01 | 0.04 | −3.59 | 0.03 | 2.23 |
| | Q_L6DqAw_l7i4d_oio | | 8.6 | −5.42 | 0 | 0 | 0.56 | 0.64 | 0.45 |
| 10560 | TYTHT_vwkoNcgkDo6o | SLC19A2 | 9.05 | −5.88 | 0 | 0 | 1.28 | 0.78 | 0.45 |
| 55437 | KntX6g6ldIoS59QsTc | ALS2CR2 | 7.53 | 11.94 | 0 | 0 | 8.27 | 1 | 2.23 |
| 160897 | oO3ZwwVSc_3vu9J4jk | GPR180 | 7.53 | 13.75 | 0 | 0 | 9.73 | 1 | 2.23 |
| 1457 | KuoAcwHd_8SVZRV_e4 | CSNK2A1 | 7.75 | 5.83 | 0 | 0 | 1.2 | 0.77 | 2.22 |
| 51341 | WMhneeR4h9_0OVBaao | ZBTB7A | 8.68 | −4.26 | 0 | 0.01 | −1.37 | 0.2 | 0.45 |
| | lgGKciOMLnrp6vuqio | | 8.95 | −5.27 | 0 | 0 | 0.32 | 0.58 | 0.45 |
| 90799 | rV1c4pSH4wyyuCveik | CCDC45 | 7.83 | 7.8 | 0 | 0 | 3.94 | 0.98 | 2.22 |
| 8683 | WVRHH3df0pXHiErjqA | SFRS9 | 7.95 | 3.11 | 0.01 | 0.03 | −3.4 | 0.03 | 2.22 |
| 79412 | xoSgE3rnPN6h_l4W6o | KREMEN2 | 8.83 | −4.69 | 0 | 0 | −0.63 | 0.35 | 0.45 |
| 80219 | ZJx.1LL_Uf3WKy.p50 | COQ10B | 7.74 | 7.99 | 0 | 0 | 4.17 | 0.98 | 2.22 |
| | 0ZIir9WP4BOSAKhPqo | | 8.7 | −5.06 | 0 | 0 | −0.02 | 0.5 | 0.45 |
| | EU0eeLoSKoiCOq.iuo | | 8.99 | −4.98 | 0 | 0 | −0.14 | 0.46 | 0.45 |
| 51306 | ipJHzne4Sz7u0y_JLI | C5orf5 | 7.86 | 5.54 | 0 | 0 | 0.75 | 0.68 | 2.22 |
| 5876 | 9aqj_SEpSiM66Nf9MU | RABGGTB | 8.05 | 5.48 | 0 | 0 | 0.66 | 0.66 | 2.21 |
| 57158 | xEO15CGnOkWIXOeOio | JPH2 | 8.95 | −4.68 | 0 | 0 | −0.64 | 0.35 | 0.45 |
| 2739 | 9ud_nfqeixbrikosBHI | GLO1 | 7.54 | 10.12 | 0 | 0 | 6.56 | 1 | 2.21 |
| 79738 | Ql3u3Sd7vJc7vyqKv8 | BBS10 | 7.97 | 6.75 | 0 | 0 | 2.54 | 0.93 | 2.21 |
| 9326 | 9pfxaT47p079KSH6rU | ZNHIT3 | 7.68 | 14.65 | 0 | 0 | 10.38 | 1 | 2.21 |
| 91894 | f0r1IXwSEDqoKqeKbo | C11orf52 | 8.77 | −5.49 | 0 | 0 | 0.67 | 0.66 | 0.45 |
| 1129 | cjfMdel0LjXXl0t1AI | CHRM2 | 9.11 | 2.45 | 0.03 | 0.07 | −4.56 | 0.01 | 2.21 |
| 60412 | B5Hh6lE34AdugKgKoo | EXOC4 | 8.63 | −5.27 | 0 | 0 | 0.32 | 0.58 | 0.45 |
| | THwCseKTqVQ6DTzV6o | | 8.73 | −5.28 | 0 | 0 | 0.35 | 0.59 | 0.45 |
| 6322 | KeyPr9TEqC91tc5Dv0 | SCML1 | 7.62 | 8.11 | 0 | 0 | 4.32 | 0.99 | 2.21 |
| 1389 | 0SXn903sACqoowf0So | CREBL2 | 9.11 | −3.26 | 0.01 | 0.03 | −3.14 | 0.04 | 0.45 |
| 152002 | 3XXsbKeSG4CVSq1P7M | C3orf21 | 7.64 | 8.25 | 0 | 0 | 4.49 | 0.99 | 2.2 |
| 115294 | 03JNI0NUINTSQXSFBU | PCMTD1 | 8.09 | 4.94 | 0 | 0 | −0.21 | 0.45 | 2.2 |
| | r56n1SL949LK9riiSo | | 8.86 | −5.47 | 0 | 0 | 0.65 | 0.66 | 0.45 |
| 25853 | fhJC1FcH7xEkTFEr3o | WDR40A | 7.52 | 9.68 | 0 | 0 | 6.1 | 1 | 2.2 |
| 6427 | 9Vj517sCOX7bkgEDp4 | SFRS2 | 8.91 | 3.02 | 0.01 | 0.03 | −3.57 | 0.03 | 2.2 |
| | 9v_itOuoo0XilKL_KU | | 8.27 | 2.81 | 0.02 | 0.05 | −3.94 | 0.02 | 2.2 |
| 6741 | BnlSXq3rAoAsS.SpCA | SSB | 7.9 | 8.09 | 0 | 0 | 4.3 | 0.99 | 2.2 |
| | 0ug6VOXstUnHainSSQ | | 9.47 | 2.24 | 0.05 | 0.1 | −4.93 | 0.01 | 2.2 |
| | KcPUJwL65sl6S7uKLU | | 7.46 | 11.44 | 0 | 0 | 7.82 | 1 | 2.2 |
| | 9roOqLqirMmr3zPC_o | | 9.36 | −2.64 | 0.02 | 0.06 | −4.23 | 0.01 | 0.45 |
| 152816 | 9VIIIdSIoJ03igJ4io | C4orf26 | 8.64 | −5.31 | 0 | 0 | 0.39 | 0.6 | 0.45 |
| 6884 | Bm5y6jpI4oIIVRv4oI | TAF13 | 7.51 | 13.91 | 0 | 0 | 9.84 | 1 | 2.2 |
| 9898 | H5zqV5H1.n1170V664 | UBAP2L | 7.75 | 4.69 | 0 | 0 | −0.63 | 0.35 | 2.2 |
| 648 | Q62B_u7vTRU0vP7irs | BMI1 | 8.08 | 2.96 | 0.01 | 0.04 | −3.68 | 0.02 | 2.2 |
| 51029 | HiiPQRRISeSh2M1On0 | FAM152A | 7.83 | 5.91 | 0 | 0 | 1.32 | 0.79 | 2.2 |
| 8487 | fp09Hj2kn5CCud4Lik | SIP1 | 7.63 | 11.75 | 0 | 0 | 8.1 | 1 | 2.2 |
| 56993 | 3g1c7OIoqKSnkn0lEg | TOMM22 | 7.64 | 5.23 | 0 | 0 | 0.26 | 0.57 | 2.19 |
| 64324 | ogOC6GkkOACSV91SVU | NSD1 | 7.56 | 14.54 | 0 | 0 | 10.3 | 1 | 2.19 |
| 221662 | Eom6S6sA66EpFAnP90 | RBM24 | 7.66 | 12.88 | 0 | 0 | 9.05 | 1 | 2.19 |
| 6845 | ip06xe99zUBf0PTnF4 | VAMP7 | 7.71 | 11.34 | 0 | 0 | 7.74 | 1 | 2.19 |
| 201725 | 314xQjiCOncNJ4hJFI | LOC201725 | 7.66 | 9.49 | 0 | 0 | 5.9 | 1 | 2.19 |
| 23512 | rd4._XejLI3Ym.N2p8 | SUZ12 | 7.85 | 4.58 | 0 | 0 | −0.82 | 0.31 | 2.19 |
| 23167 | 03kIj3koHq7kRrvslI | EFR3A | 8.07 | 3.06 | 0.01 | 0.03 | −3.49 | 0.03 | 2.19 |
| 79016 | 05GRvqiC2lfqN.d_LQ | DDA1 | 7.73 | 5.97 | 0 | 0 | 1.42 | 0.8 | 2.19 |
| | iqLvKA4QLopJE7_uqo | | 8.62 | −5.02 | 0 | 0 | −0.08 | 0.48 | 0.46 |
| 29968 | Ek9TyQi_xVdVLfZfXc | PSAT1 | 7.47 | 14.52 | 0 | 0 | 10.28 | 1 | 2.19 |
| 7170 | N6fAOx3X.O63f6pY_o | TPM3 | 8.86 | 2.71 | 0.02 | 0.05 | −4.11 | 0.02 | 2.19 |
| | 0HelXOmuilLH_QBRgE | | 8.04 | 4 | 0 | 0.01 | −1.82 | 0.14 | 2.19 |
| 9231 | QeyHPdCs8HZIV3qrKo | DLG5 | 8.77 | −6.97 | 0 | 0 | 2.84 | 0.94 | 0.46 |
| 4610 | ifPek3yF1EVuIDXoio | MYCL1 | 8.9 | −4.78 | 0 | 0 | −0.48 | 0.38 | 0.46 |
| | 9VKA3NT6p_LoLjqrCo | | 8.82 | −5.46 | 0 | 0 | 0.62 | 0.65 | 0.46 |
| 65983 | 02.s3vpcuhJ7l65F3o | GRAMD3 | 8.26 | 4.67 | 0 | 0 | −0.67 | 0.34 | 2.19 |
| | NUEsrgJ8kucTvT9Emo | | 8.91 | −4.69 | 0 | 0 | −0.63 | 0.35 | 0.46 |
| 5430 | xB8qsvXnuLrxJ412oI | POLR2A | 7.89 | 3.42 | 0.01 | 0.02 | −2.86 | 0.05 | 2.19 |
| | 635TNDQHzIiEDHqmqo | | 8.49 | −5.9 | 0 | 0 | 1.31 | 0.79 | 0.46 |
| 55973 | BU_IInUUkheOXOBERI | BCAP29 | 7.79 | 8.37 | 0 | 0 | 4.63 | 0.99 | 2.19 |
| 343413 | xIF0CKLglK3ni9FuqI | FCRL6 | 8.65 | −5.34 | 0 | 0 | 0.44 | 0.61 | 0.46 |
| 22806 | ihNn9f60K_kiQqzAqo | IKZF3 | 8.63 | −7.09 | 0 | 0 | 3.01 | 0.95 | 0.46 |
| 94107 | KlKvi1atqlqo_WmTSo | TMEM203 | 9.03 | −3.49 | 0 | 0.02 | −2.72 | 0.06 | 0.46 |
| 1843 | EkgiAodLu41r9._dlU | DUSP1 | 7.62 | 7.56 | 0 | 0 | 3.63 | 0.97 | 2.18 |
| 147339 | iOR_kvDo3nvnou4m6E | C18orf25 | 7.63 | 13.1 | 0 | 0 | 9.23 | 1 | 2.18 |
| 9631 | ud5ejnv7rxfvidS3OI | NUP155 | 8.39 | −8.88 | 0 | 0 | 5.22 | 0.99 | 0.46 |
| 819 | ZrdJSVyIeffu.u097U | CAMLG | 8.5 | 4.89 | 0 | 0 | −0.29 | 0.43 | 2.18 |
| | rNJREExcHLrf6TlSuo | | 8.82 | −6.11 | 0 | 0 | 1.61 | 0.83 | 0.46 |
| 134492 | KmjX6uQhQOSICfE8iI | NUDCD2 | 7.53 | 11.78 | 0 | 0 | 8.13 | 1 | 2.18 |
| | frfk._kxUkLOxChk6o | | 8.89 | −5.11 | 0 | 0 | 0.06 | 0.52 | 0.46 |
| 55320 | 6fhMoz.V3pxE9FxX70 | C14orf106 | 7.8 | 6.56 | 0 | 0 | 2.28 | 0.91 | 2.18 |
| 84992 | En_ZM0p8oe6inwvkjk | PIGY | 7.69 | 8.75 | 0 | 0 | 5.08 | 0.99 | 2.18 |
| 6303 | EpBIouKLnnsjhBdT3M | SAT1 | 8.34 | 6.8 | 0 | 0 | 2.61 | 0.93 | 2.17 |
| | Tlmcdek7o0UIxD9614 | | 7.97 | 5.93 | 0 | 0 | 1.35 | 0.79 | 2.17 |
| 51259 | T4kaIl0766v1IuO4CU | MGC13379 | 7.57 | 7.75 | 0 | 0 | 3.87 | 0.98 | 2.17 |
| | 9903iU_Col3Td1FiKo | | 8.62 | −5.01 | 0 | 0 | −0.1 | 0.48 | 0.46 |
| 10463 | 6vSskuJPvTOIOmnq40 | SLC30A9 | 7.94 | 6.42 | 0 | 0 | 2.08 | 0.89 | 2.17 |
| 2054 | KqF5dW47VF.X0K3tM4 | STX2 | 7.86 | 5.34 | 0 | 0 | 0.43 | 0.61 | 2.17 |
| | KK8G73dR7vnXqI6IKo | | 8.9 | −4.69 | 0 | 0 | −0.63 | 0.35 | 0.46 |
| 27131 | xcOlnqq4qa7vs7f6u8 | SNX5 | 8.3 | 2.26 | 0.04 | 0.09 | −4.88 | 0.01 | 2.17 |
| | cYK0pDEsrIu8p6ogJo | | 8.53 | −5.87 | 0 | 0 | 1.27 | 0.78 | 0.46 |
| 65991 | lXn1UR3l3XhN6t3q84 | FUNDC2 | 8.11 | −8.75 | 0 | 0 | 5.08 | 0.99 | 0.46 |
| 10929 | rniefXv994_deqAEZc | SFRS2B | 7.78 | 8.16 | 0 | 0 | 4.38 | 0.99 | 2.17 |
| 55970 | TkeV81_7Tef.b3mM5U | GNG12 | 7.77 | 10.62 | 0 | 0 | 7.05 | 1 | 2.17 |
| | QbvcDk5F4sAo3qeS3I | | 7.5 | 11.6 | 0 | 0 | 7.97 | 1 | 2.17 |
| | TRo0eSXUXVL0ID3Tt0 | | 8.12 | 2.95 | 0.01 | 0.04 | −3.69 | 0.02 | 2.16 |
| 29068 | BTrREwL5LygKjqSoAE | ZBTB44 | 7.82 | 6.22 | 0 | 0 | 1.78 | 0.86 | 2.16 |
| | in1cVdGmFxJCLNAgEI | | 8.12 | −7.4 | 0 | 0 | 3.42 | 0.97 | 0.46 |
| 6830 | xQlP_B95XRaHep7h6o | SUPT6H | 8.88 | −4.67 | 0 | 0 | −0.67 | 0.34 | 0.46 |
| | 3l_CD7lFRedRyDl4B4 | | 8.13 | −7.27 | 0 | 0 | 3.25 | 0.96 | 0.46 |
| | opUM.FCiF9d3ljrWio | | 8.53 | −6.48 | 0 | 0 | 2.16 | 0.9 | 0.46 |
| 4193 | QVV9TRPkEjvqV9uIPo | MDM2 | 7.71 | 7.85 | 0 | 0 | 3.99 | 0.98 | 2.16 |
| | 6Sd7n55t.4kPRcF3UI | | 8.09 | −5.75 | 0 | 0 | 1.08 | 0.75 | 0.46 |
| 6138 | QrxUd7UdUynEgAtEJk | RPL15 | 8.18 | 3.81 | 0 | 0.01 | −2.16 | 0.1 | 2.16 |
| 64065 | oep3NMyEp94y.kHsJI | PERP | 8.05 | 3.35 | 0.01 | 0.02 | −2.97 | 0.05 | 2.16 |
| | cx3O_VLnobkOnuN2Z0 | | 8.05 | 2.47 | 0.03 | 0.07 | −4.53 | 0.01 | 2.16 |
| | Nfp52erfo7avYUfpY4 | | 8.23 | 2.45 | 0.03 | 0.07 | −4.57 | 0.01 | 2.16 |
| | KU01y6gcB_N8qOMCFY | | 7.53 | 11.76 | 0 | 0 | 8.11 | 1 | 2.16 |
| | ckgBTieA3cSgUueqyo | | 8.83 | −5.41 | 0 | 0 | 0.54 | 0.63 | 0.46 |
| 51317 | 3tLitW4t.uLX7tNvak | PHF21A | 8.07 | 4.31 | 0 | 0.01 | −1.28 | 0.22 | 2.16 |
| 169200 | uXr46X666D.v0lIpx0 | TMEM64 | 7.92 | 8.57 | 0 | 0 | 4.87 | 0.99 | 2.16 |
| | fuy06IIuvlI7zOqgqo | | 8.51 | −5.14 | 0 | 0 | 0.11 | 0.53 | 0.46 |
| 3229 | 6I55cEvV10ClF_ue6o | HOXC13 | 8.89 | −4.93 | 0 | 0 | −0.22 | 0.44 | 0.46 |
| 54836 | Nwv1cXjpVd4TAeRF6o | BSPRY | 9 | −4.78 | 0 | 0 | −0.47 | 0.38 | 0.46 |
| 3015 | oHcEntS7649S6Hs3e4 | H2AFZ | 7.6 | 6.61 | 0 | 0 | 2.35 | 0.91 | 2.15 |
| 3516 | lTFK4xz0AFW_3V5C54 | RBPJ | 7.78 | 5.4 | 0 | 0 | 0.54 | 0.63 | 2.15 |
| 3646 | uJK0inXB6kHQj3p9x4 | EIF3E | 8.59 | 2.75 | 0.02 | 0.05 | −4.04 | 0.02 | 2.15 |
| | f0TO9M5Vzv6qzR0v9U | | 7.58 | 9.9 | 0 | 0 | 6.34 | 1 | 2.15 |
| | EoF7BzxdM6QCof6o6o | | 8.55 | −5.9 | 0 | 0 | 1.31 | 0.79 | 0.46 |
| 3189 | ZkrzZCO3yLKvCvdLhE | HNRPH3 | 7.62 | 7.25 | 0 | 0 | 3.22 | 0.96 | 2.15 |
| 8773 | Tk3gR9AntLuz6.RQwU | SNAP23 | 7.7 | 7.25 | 0 | 0 | 3.22 | 0.96 | 2.15 |
| | ETPlWZcy.7wt8V7D.4 | | 7.7 | 10.19 | 0 | 0 | 6.63 | 1 | 2.15 |
| 23560 | Q_wieokBG.OLu2g1bk | GTPBP4 | 7.81 | 4.52 | 0 | 0.01 | −0.92 | 0.28 | 2.15 |
| 6382 | 0HmH95ei7OHkTh2quo | SDC1 | 8.73 | −4.99 | 0 | 0 | −0.13 | 0.47 | 0.47 |
| 10135 | WbfQoVL541QQCtQAqU | NAMPT | 7.44 | 10.43 | 0 | 0 | 6.87 | 1 | 2.15 |
| 286148 | N5KS7F0r4d7E3gy4tE | DPY19L4 | 8.19 | 4.46 | 0 | 0.01 | −1.01 | 0.27 | 2.15 |
| 618 | rooyfiVKL2IXl6kMyY | BCYRN1 | 8.83 | 2.42 | 0.03 | 0.08 | −4.61 | 0.01 | 2.15 |
| 79053 | rcXn6Xql3oLee4P7PU | ALG8 | 7.74 | 6.49 | 0 | 0 | 2.17 | 0.9 | 2.15 |
| 29942 | H4qqKjD9BUeXfFIBP8 | PURG | 8.19 | −5.06 | 0 | 0 | −0.02 | 0.49 | 0.47 |
| 9184 | lSy3hs.Vfe1XLCVL54 | BUB3 | 9.35 | 2.41 | 0.03 | 0.08 | −4.64 | 0.01 | 2.14 |
| 4233 | Bt3FKtCJOBk0MgIHao | MET | 8.9 | −6.22 | 0 | 0 | 1.79 | 0.86 | 0.47 |
| 5437 | 9ppN.B6XUdDqAeHZe4 | POLR2H | 7.65 | 6.2 | 0 | 0 | 1.76 | 0.85 | 2.14 |
| | Nq6KpKKhA6I4NTwA_o | | 8.75 | −4.15 | 0 | 0.01 | −1.55 | 0.18 | 0.47 |
| | 0PYCACDlDOACICSAiA | | 8.09 | −6.62 | 0 | 0 | 2.36 | 0.91 | 0.47 |
| 201633 | EAyod9cqgAqqeouMio | VSTM3 | 8.62 | −5.46 | 0 | 0 | 0.63 | 0.65 | 0.47 |
| | leHovS65ARJ3dRBd_o | | 8.59 | −5.47 | 0 | 0 | 0.64 | 0.65 | 0.47 |
| 7117 | upUK7Xkp7Dkvw0i5T8 | TMSL3 | 10.1 | 2.82 | 0.02 | 0.04 | −3.92 | 0.02 | 2.14 |
| 84447 | ulIdet.UvyAKd7lCio | SYVN1 | 8.75 | −5.92 | 0 | 0 | 1.33 | 0.79 | 0.47 |
| 56957 | ZUqFwiAskqgr_u7qro | OTUD7B | 8.63 | −5.65 | 0 | 0 | 0.93 | 0.72 | 0.47 |
| 4735 | H_cuHCVZM7i4Cx9KRk | 2-Sep | 8.11 | 2.44 | 0.03 | 0.07 | −4.59 | 0.01 | 2.14 |
| 51528 | TESrDPN5xHoKWcoJXY | C14orf100 | 7.83 | 9.58 | 0 | 0 | 5.99 | 1 | 2.14 |
| 253260 | xGecTP6K967_ytHgSE | RICTOR | 7.92 | 6.81 | 0 | 0 | 2.62 | 0.93 | 2.14 |
| | WFHk7A3uozJwJHnyqI | | 8.37 | −4.74 | 0 | 0 | −0.55 | 0.37 | 0.47 |
| 23484 | fkece.zUfpRIf.cEnk | LEPROTL1 | 8.01 | 4.17 | 0 | 0.01 | −1.52 | 0.18 | 2.14 |
| 277 | 38nxvNxSJFup7x_X4s | AMY1B | 7.8 | 5.13 | 0 | 0 | 0.09 | 0.52 | 2.14 |
| 1774 | oHqHSBUEEqEgh0ZX6o | DNASE1L1 | 8.57 | −5.75 | 0 | 0 | 1.07 | 0.75 | 0.47 |
| 51304 | Q_CiuuOujIrgorKi5U | ZDHHC3 | 7.97 | 2.4 | 0.04 | 0.08 | −4.65 | 0.01 | 2.13 |
| 79768 | Be4.iK4vPwilL.ShOo | C15orf29 | 8.78 | 5.52 | 0 | 0 | 0.72 | 0.67 | 2.13 |
| 8766 | x_3fmudOO7qkRoKT54 | RAB11A | 8.16 | 4.77 | 0 | 0 | −0.5 | 0.38 | 2.13 |
| 55837 | TVLTuR4O9R4LpLcYQo | EAPP | 7.57 | 14.07 | 0 | 0 | 9.96 | 1 | 2.13 |
| 51083 | x4v3t.fk3QIpa5XYWU | GAL | 7.53 | 14.05 | 0 | 0 | 9.94 | 1 | 2.13 |
| | 0uwlcH3gpHzS3SnKqI | | 8.68 | −5.55 | 0 | 0 | 0.77 | 0.68 | 0.47 |
| | 0bECSHlZA2FrrAkVKA | | 7.83 | 6.16 | 0 | 0 | 1.69 | 0.84 | 2.13 |
| 285671 | lYKbgI6cSCSrAx0ouo | RNF180 | 8.71 | −4.92 | 0 | 0 | −0.24 | 0.44 | 0.47 |
| 56672 | fl..B_KuyK11Gfv5eA | C11orf17 | 7.87 | 5.48 | 0 | 0 | 0.65 | 0.66 | 2.13 |
| 6119 | Z3eg_6C6_giwWUKUU0 | RPA3 | 7.68 | 12.47 | 0 | 0 | 8.72 | 1 | 2.13 |
| | 6iiQn4jA_8MPPvtS6o | | 8.73 | −4.9 | 0 | 0 | −0.29 | 0.43 | 0.47 |
| | 95SVw6USP94Y8sLPz0 | | 7.9 | 7.31 | 0 | 0 | 3.31 | 0.96 | 2.13 |
| | TJItHkgl6NfkwX7Yuo | | 9.09 | −3.6 | 0 | 0.02 | −2.52 | 0.07 | 0.47 |
| | ov6sop_dyss.4KClSo | | 8.86 | −4.03 | 0 | 0.01 | −1.76 | 0.15 | 0.47 |
| | rvuijuoIHSDVDEycuo | | 8.63 | −4.61 | 0 | 0 | −0.77 | 0.32 | 0.47 |
| 60481 | udET_KS60BKwJc47u0 | ELOVL5 | 7.84 | 4.15 | 0 | 0.01 | −1.55 | 0.18 | 2.12 |
| 4267 | opTzwrXsHQO0FUWKRU | CD99 | 7.53 | 8.37 | 0 | 0 | 4.63 | 0.99 | 2.12 |
| 9076 | Zuc3vS45XSJ357yekk | CLDN1 | 7.51 | 16.24 | 0 | 0 | 11.42 | 1 | 2.12 |
| 55532 | HmgCRr0V9CIEksod6o | SLC30A10 | 8.84 | −4.64 | 0 | 0 | −0.72 | 0.33 | 0.47 |
| 911 | BkIOUr766n4cQErOKo | CD1C | 8.61 | −3.67 | 0 | 0.02 | −2.41 | 0.08 | 0.47 |
| | lr8kqE4rof_H6snvQ4 | | 7.77 | 8.4 | 0 | 0 | 4.67 | 0.99 | 2.12 |
| | u4dX8Dt1ICUMA4jIio | | 8.71 | −5.19 | 0 | 0 | 0.19 | 0.55 | 0.47 |
| | Nl9A0UHu0FB5d8_X54 | | 8.03 | −7.03 | 0 | 0 | 2.92 | 0.95 | 0.47 |
| | 9cwWXXRJunW5.v7sqo | | 8.79 | −5.28 | 0 | 0 | 0.34 | 0.58 | 0.47 |
| 57826 | No2Sg0qgLj.Arr91JE | RAP2C | 8.07 | 7.66 | 0 | 0 | 3.76 | 0.98 | 2.12 |
| | 0iTXhh1P196dVES3xE | | 8.03 | −6.12 | 0 | 0 | 1.64 | 0.84 | 0.47 |
| | uopIhU54vp4iNLJQno | | 7.68 | 7.35 | 0 | 0 | 3.36 | 0.97 | 2.12 |
| 2957 | u..VQ35rj_rRXWrvMs | GTF2A1 | 7.45 | 4.98 | 0 | 0 | −0.15 | 0.46 | 2.12 |
| 2258 | TmCOYpTxe.jV7aAXpE | FGF13 | 8.14 | −6.59 | 0 | 0 | 2.32 | 0.91 | 0.47 |
| | Qi_4HrqWzsEnhQbgjE | | 10 | 2.41 | 0.03 | 0.08 | −4.64 | 0.01 | 2.11 |
| 2697 | Wi_JLf_i4UkH_O.kcI | GJA1 | 7.59 | 13.75 | 0 | 0 | 9.72 | 1 | 2.11 |
| 10672 | NiCtd68t3QPinvyoDU | GNA13 | 7.7 | 7.55 | 0 | 0 | 3.61 | 0.97 | 2.11 |
| | 135d4NwRAbp8vfkgkk | | 7.95 | −7.71 | 0 | 0 | 3.83 | 0.98 | 0.47 |
| | KgR4AvymnirntuAsqo | | 8.66 | −4.8 | 0 | 0 | −0.45 | 0.39 | 0.47 |
| 3998 | NBeUcT3r8Ty8fTnp6o | LMAN1 | 8.97 | −5.04 | 0 | 0 | −0.04 | 0.49 | 0.47 |
| 644914 | f7pnW4DyG80gtR4H3o | LOC644914 | 7.56 | 7.67 | 0 | 0 | 3.77 | 0.98 | 2.11 |
| | 3dUD1VNX3oikISLS6o | | 8.68 | −4.89 | 0 | 0 | −0.3 | 0.42 | 0.47 |
| | ogG4U9fq9fTuojkh6k | | 8.11 | −7.63 | 0 | 0 | 3.72 | 0.98 | 0.47 |
| 55207 | WooPoHIO7h3uqHkyv4 | ARL8B | 8.22 | 2.43 | 0.03 | 0.07 | −4.61 | 0.01 | 2.11 |
| 1973 | 02tiuhr_lsCu6c8E64 | EIF4A1 | 7.44 | 4.91 | 0 | 0 | −0.26 | 0.44 | 2.11 |
| | xuQ04dHkl0B14nQuNc | | 8.65 | 2.6 | 0.02 | 0.06 | −4.31 | 0.01 | 2.11 |
| 5033 | QXkwQR6l98HAOdFHyU | P4HA1 | 8.07 | 4.32 | 0 | 0.01 | −1.26 | 0.22 | 2.11 |
| | 6TXndBzBPppp6kildc | | 7.5 | 7.22 | 0 | 0 | 3.19 | 0.96 | 2.11 |
| 4001 | QqKsDAUe5t_uw.gj5U | LMNB1 | 7.75 | 5.96 | 0 | 0 | 1.4 | 0.8 | 2.11 |
| 9553 | HHlSu60U713Wa33_UI | MRPL33 | 7.57 | 7.73 | 0 | 0 | 3.85 | 0.98 | 2.11 |
| | 3.VCCdEjf9ScTXniKo | | 8.66 | −4.61 | 0 | 0 | −0.77 | 0.32 | 0.48 |
| | HK6nVduR.Pl6kv_fjc | | 7.84 | 4.81 | 0 | 0 | −0.42 | 0.4 | 2.1 |
| 58517 | KEReiKE.gBHvfFdgV4 | RBM25 | 7.98 | 5.03 | 0 | 0 | −0.06 | 0.48 | 2.1 |
| 50831 | BkA6hCdSI1BR4pUQio | TAS2R3 | 8.71 | −5.29 | 0 | 0 | 0.35 | 0.59 | 0.48 |
| | ifeztLST8OFFx7uRJE | | 7.71 | 4.78 | 0 | 0 | −0.47 | 0.38 | 2.1 |
| | BdPveyIyAM2RTKKqIo | | 8.48 | −5.72 | 0 | 0 | 1.03 | 0.74 | 0.48 |
| | 3fYBMgAaDHiN66qlpo | | 8.17 | −4.81 | 0 | 0 | −0.43 | 0.39 | 0.48 |
| 195827 | cntX1SX_KLyoJMCqn4 | C9orf21 | 7.4 | 11.96 | 0 | 0 | 8.29 | 1 | 2.1 |
| | Knkg_KupfIXrO6KIg4 | | 7.59 | 13.12 | 0 | 0 | 9.24 | 1 | 2.1 |
| 9373 | oOeyPzXA.nskuqCKho | PLAA | 8.64 | −5.34 | 0 | 0 | 0.44 | 0.61 | 0.48 |
| 55330 | rl7t9Sdw0gJ5_2Kk6o | CNO | 8.87 | −3.85 | 0 | 0.01 | −2.07 | 0.11 | 0.48 |
| | 9enQrQIwqeqr6N6feo | | 9.12 | −2.89 | 0.01 | 0.04 | −3.8 | 0.02 | 0.48 |
| 8890 | Qvexe_9xi0IkuFLhqo | EIF2B4 | 8.73 | −5.85 | 0 | 0 | 1.23 | 0.77 | 0.48 |
| | 0TgoKQkv0WjLhMU2UU | | 7.51 | 10.56 | 0 | 0 | 6.99 | 1 | 2.09 |
| 5062 | iWC39U9H3cgJ5QhIpI | PAK2 | 7.89 | 5.18 | 0 | 0 | 0.17 | 0.54 | 2.09 |
| | WerLIDdICKiAwA6oqo | | 8.57 | −5.32 | 0 | 0 | 0.41 | 0.6 | 0.48 |
| 10289 | NeUv3for6T7AooEnwo | EIF1B | 7.46 | 10.96 | 0 | 0 | 7.38 | 1 | 2.09 |
| 64430 | QH4ofdRFVPSOXiBJwk | C14orf135 | 7.82 | 6.49 | 0 | 0 | 2.18 | 0.9 | 2.09 |
| 400509 | QieqL0IUl6kEw4j9J0 | RUNDC2B | 8.36 | 2.48 | 0.03 | 0.07 | −4.51 | 0.01 | 2.09 |
| 10178 | cCCnkIqoIqjypOhIKo | ODZ1 | 8.49 | −5.95 | 0 | 0 | 1.38 | 0.8 | 0.48 |
| 94081 | 0oqa16BQk1GjHiq4T8 | SFXN1 | 7.56 | 7.68 | 0 | 0 | 3.78 | 0.98 | 2.09 |
| | Hl6BOmsEH3T.Q_PR6o | | 8.81 | −5.02 | 0 | 0 | −0.08 | 0.48 | 0.48 |
| 6801 | ESe9EwJF6QJP8XvKqI | STRN | 8.73 | −5.02 | 0 | 0 | −0.07 | 0.48 | 0.48 |
| 2009 | BkjiHsv5I6boJPTSqI | EML1 | 8.58 | −5.31 | 0 | 0 | 0.39 | 0.6 | 0.48 |
| | TX6ABK4gejICTsUjio | | 8.79 | −4.62 | 0 | 0 | −0.74 | 0.32 | 0.48 |
| 9782 | HpTDXI5GfcPTsXkTuE | MATR3 | 8.73 | 2.75 | 0.02 | 0.05 | −4.04 | 0.02 | 2.08 |
| 523 | 9jjkvez8_57t61wuiU | ATP6V1A | 8.08 | 3.99 | 0 | 0.01 | −1.84 | 0.14 | 2.08 |
| 6917 | HCAgXkPE2SBJXikIgg | TCEA1 | 7.53 | 9.64 | 0 | 0 | 6.07 | 1 | 2.08 |
| 389674 | cA.6KSCd6VU666KUsc | HNRPA1P4 | 7.98 | 3.26 | 0.01 | 0.03 | −3.13 | 0.04 | 2.08 |
| 8365 | NGR38afa6cKdefnkd8 | HIST1H4H | 7.6 | 5.4 | 0 | 0 | 0.53 | 0.63 | 2.08 |
| | 6jh6c.oAXq5x5Qers0 | | 7.67 | 9.15 | 0 | 0 | 5.53 | 1 | 2.08 |
| 143187 | lks.ysTbiOol5PVKqY | VTI1A | 8.62 | −5.27 | 0 | 0 | 0.32 | 0.58 | 0.48 |
| | cTs7slK10AOyBIijio | | 8.57 | −5.14 | 0 | 0 | 0.12 | 0.53 | 0.48 |
| | oj53EpP3p1uNU4XQKo | | 8.86 | −4.65 | 0 | 0 | −0.7 | 0.33 | 0.48 |
| 9559 | TjqodKKF7gk7Lzcjeo | VPS26A | 7.79 | 12.08 | 0 | 0 | 8.39 | 1 | 2.07 |
| 103910 | NfdIR9VRXTIF75OQfI | MRLC2 | 7.62 | 8.67 | 0 | 0 | 4.99 | 0.99 | 2.07 |
| | rAPOk5b8kgeCOoI.NI | | 8.25 | −4.94 | 0 | 0 | −0.22 | 0.45 | 0.48 |
| 142 | uFAn28g7eXx6.VSoKA | PARP1 | 8.15 | 3.41 | 0.01 | 0.02 | −2.87 | 0.05 | 2.07 |
| 54453 | TUv5K5EBzirfs1GwRI | RIN2 | 7.65 | 6.94 | 0 | 0 | 2.81 | 0.94 | 2.07 |
| | H6dQAFfgAgFZW1Q16o | | 8.52 | −5.02 | 0 | 0 | −0.09 | 0.48 | 0.48 |
| 6399 | TnqJL5KUS4Lu_fe0fw | TRAPPC2 | 7.45 | 7.55 | 0 | 0 | 3.62 | 0.97 | 2.07 |
| 79650 | 3KT3iqQbooKlKjk6j8 | C16orf57 | 7.76 | 6.05 | 0 | 0 | 1.53 | 0.82 | 2.07 |
| | TJEoiL6ciul9_Iokuo | | 8.91 | −4.67 | 0 | 0 | −0.66 | 0.34 | 0.48 |
| 27075 | WukXoPx7PT7ake1Huk | TSPAN13 | 8.04 | 7.72 | 0 | 0 | 3.84 | 0.98 | 2.07 |
| | Nv6rtD.c0GQEXyrFSE | | 7.77 | 5.34 | 0 | 0 | 0.43 | 0.61 | 2.06 |
| | K3SOEdMkRF34UkgJRI | | 8.02 | −7.42 | 0 | 0 | 3.44 | 0.97 | 0.48 |
| 7414 | HVr31Lr_IR6.Efzlo4 | VCL | 7.67 | 7.48 | 0 | 0 | 3.53 | 0.97 | 2.06 |
| 134553 | B5HriTdePUfH.dXuBI | C5orf24 | 7.55 | 9.59 | 0 | 0 | 6.01 | 1 | 2.06 |
| 93487 | iTV_PXV47rAekR1Krc | MAPK1IP1L | 7.97 | 9.14 | 0 | 0 | 5.52 | 1 | 2.06 |
| 10653 | xp59et6So6v5.oDXco | SPINT2 | 8.63 | 2.91 | 0.01 | 0.04 | −3.76 | 0.02 | 2.06 |
| 7009 | 04TlFWDp0f0eKOe13g | TEGT | 7.88 | 3.9 | 0 | 0.01 | −2 | 0.12 | 2.06 |
| | 3stASAIQeJJA0kBDAU | | 8.09 | −5.94 | 0 | 0 | 1.36 | 0.8 | 0.49 |
| 9867 | iJUrcDsOvr8_9zBVJU | PJA2 | 8.44 | 2.68 | 0.02 | 0.05 | −4.17 | 0.02 | 2.06 |
| 7157 | ce4DnpP5FxdEi5PuKs | TP53 | 7.7 | 7.58 | 0 | 0 | 3.65 | 0.97 | 2.06 |
| 160287 | 9Sq4lHuXpfJ.j8s1I4 | LDHAL6A | 7.93 | 3.97 | 0 | 0.01 | −1.87 | 0.13 | 2.06 |
| | 0SXouANerHh.iKLi7k | | 7.79 | 7.47 | 0 | 0 | 3.51 | 0.97 | 2.06 |
| | Qh4KuYuereTu.f1NKo | | 8.48 | −5.44 | 0 | 0 | 0.59 | 0.64 | 0.49 |
| 57495 | lQjITr15XQifS7Irio | KIAA1239 | 8.72 | −4.17 | 0 | 0.01 | −1.52 | 0.18 | 0.49 |
| 401494 | liEl6uEi4h3N0AiCFc | PTPLAD2 | 8.13 | 2.6 | 0.02 | 0.06 | −4.31 | 0.01 | 2.05 |
| 64746 | ZVJ0yu9Me8TUgT_0p0 | ACBD3 | 7.76 | 5.39 | 0 | 0 | 0.51 | 0.63 | 2.05 |
| 7168 | cIjQrX9YQngop4h2p4 | TPM1 | 8.69 | 3.38 | 0.01 | 0.02 | −2.92 | 0.05 | 2.05 |
| 29766 | l1KiIq623uT1K8eR_0 | TMOD3 | 7.74 | 12.6 | 0 | 0 | 8.82 | 1 | 2.05 |
| 2665 | 6cFAVSEyotP6ICXs7o | GDI2 | 7.85 | 7.84 | 0 | 0 | 3.99 | 0.98 | 2.05 |
| 29887 | lsC9OU1KT8ImNdNX0k | SNX10 | 7.85 | 4.36 | 0 | 0.01 | −1.19 | 0.23 | 2.05 |
| 10097 | ckvq9KgOo_H6X0p1.o | ACTR2 | 7.76 | 8.82 | 0 | 0 | 5.17 | 0.99 | 2.05 |
| 4609 | xTXtbUJIokAnT94Ioc | MYC | 7.61 | 10.63 | 0 | 0 | 7.07 | 1 | 2.05 |
| | NekQ0sOQFKr089cU10 | | 8.09 | −4.37 | 0 | 0.01 | −1.18 | 0.23 | 0.49 |
| 7913 | 3ei_qd_n9Iv68NLvj0 | DEK | 7.97 | 2.55 | 0.03 | 0.06 | −4.39 | 0.01 | 2.05 |
| | HeLKWh27p0Zew1SOe8 | | 8.43 | 2.38 | 0.04 | 0.08 | −4.68 | 0.01 | 2.05 |
| | ZrXUBVXCAVoPMV6oGo | | 8.63 | −5.36 | 0 | 0 | 0.46 | 0.61 | 0.49 |
| 92935 | fV_BS7XIUjj.5U7s1U | MARS2 | 7.73 | 7.09 | 0 | 0 | 3.01 | 0.95 | 2.05 |
| 55664 | TkO.EIHr496ik3nEt0 | CDC37L1 | 7.7 | 7.67 | 0 | 0 | 3.77 | 0.98 | 2.05 |
| 10985 | KV7SOAOPRJ3pL1Qt6o | GCN1L1 | 8.64 | −9.39 | 0 | 0 | 5.8 | 1 | 0.49 |
| | 9opK4kt6gT9AIoCqaI | | 8.77 | −5.79 | 0 | 0 | 1.14 | 0.76 | 0.49 |
| 55142 | QLTjRlHmcSXqYRLCFc | CEP27 | 8.28 | 2.31 | 0.04 | 0.09 | −4.81 | 0.01 | 2.04 |
| 595 | 3aZ9UkUE7BaTv1JIIQ | CCND1 | 7.93 | 2.26 | 0.05 | 0.09 | −4.89 | 0.01 | 2.04 |
| | BZ4_BIwMYB5TTT_yA | | 7.58 | 7.71 | 0 | 0 | 3.83 | 0.98 | 2.04 |
| 85403 | 06QoKyj94B0pfBQyvo | EAF1 | 8.17 | 3.26 | 0.01 | 0.03 | −3.14 | 0.04 | 2.04 |
| 284418 | cHuKBGlFCkiiMSig2o | FAM71E2 | 8.88 | −4.1 | 0 | 0.01 | −1.64 | 0.16 | 0.49 |
| 282809 | roortRPzruzKwTqgDk | WDR51B | 7.44 | 9.32 | 0 | 0 | 5.71 | 1 | 2.04 |
| | 6noAkCKVCgSRIV4euo | | 8.46 | −5.11 | 0 | 0 | 0.06 | 0.52 | 0.49 |
| 26225 | 9d7vd7PscFBdUDZ4nk | ARL5A | 7.48 | 6.93 | 0 | 0 | 2.8 | 0.94 | 2.04 |
| | 6KES0BTJFBzeX.Ueqo | | 8.49 | −5.94 | 0 | 0 | 1.37 | 0.8 | 0.49 |
| | fn4ygnHqVzgaKHC6Co | | 8.66 | −5.36 | 0 | 0 | 0.47 | 0.62 | 0.49 |
| 253827 | 3oLk6h.V3eR1J3x7FM | MSRB3 | 7.77 | 3.48 | 0.01 | 0.02 | −2.73 | 0.06 | 2.04 |
| 10472 | f6oKq8shzuj5pJIp5I | ZNF238 | 7.36 | 9.75 | 0 | 0 | 6.18 | 1 | 2.04 |
| 6434 | QStf58UCilQ.R0Dv_I | SFRS10 | 8.02 | 4.3 | 0 | 0.01 | −1.3 | 0.21 | 2.04 |
| 64924 | ETkvnq7PP0d_6..pdE | SLC30A5 | 7.98 | 6 | 0 | 0 | 1.45 | 0.81 | 2.04 |
| 79230 | TZHS0N9EKRCIvTGqio | ZNF557 | 8.64 | −5.18 | 0 | 0 | 0.17 | 0.54 | 0.49 |
| 9516 | rkT.iTt6sU65o5_oFI | LITAF | 7.68 | 8.32 | 0 | 0 | 4.58 | 0.99 | 2.04 |
| 23387 | ikV1JBBI4CUuJccFuU | KIAA0999 | 7.36 | 9 | 0 | 0 | 5.36 | 1 | 2.04 |
| | iuVLv_O74jhyOzpPuo | | 9.23 | −2.82 | 0.02 | 0.04 | −3.92 | 0.02 | 0.49 |
| | 3eqMsORAOuieCKqOqY | | 8.47 | −5.35 | 0 | 0 | 0.45 | 0.61 | 0.49 |
| | Ts.vi6de5le570K56o | | 8.69 | −5 | 0 | 0 | −0.12 | 0.47 | 0.49 |
| 113115 | 6XS.03dSUW7.XVsQUo | FAM54A | 7.72 | 11.66 | 0 | 0 | 8.02 | 1 | 2.04 |
| 23345 | usS.nAlC.7_z4.f5Pw | SYNE1 | 7.51 | 5.96 | 0 | 0 | 1.4 | 0.8 | 2.04 |
| 8543 | KcV08EPJKkelIv7Fe4 | LMO4 | 7.78 | 10.15 | 0 | 0 | 6.59 | 1 | 2.04 |
| 6921 | fmunmoeGIBxwL16oJA | TCEB1 | 7.55 | 7.28 | 0 | 0 | 3.26 | 0.96 | 2.04 |
| 24139 | iWrqCKi0TZ.qjU_u.o | EML2 | 7.71 | 5.51 | 0 | 0 | 0.7 | 0.67 | 2.04 |
| 116985 | oIvY4d4cY4rVIoqlqo | CENTD2 | 8.77 | −5.5 | 0 | 0 | 0.7 | 0.67 | 0.49 |
| 153339 | 95SDsvlNI.j7gQoeHk | TMEM167 | 7.65 | 8.41 | 0 | 0 | 4.68 | 0.99 | 2.04 |
| | TSATTUttFVDl0FSoSo | | 8.65 | −4.67 | 0 | 0 | −0.66 | 0.34 | 0.49 |
| 51802 | 3TB3J5Vv37ggMgxWlE | ACCN5 | 7.97 | −6.07 | 0 | 0 | 1.56 | 0.83 | 0.49 |
| 55328 | H76Qo7ojPo9X7kuWuo | C10orf59 | 8.62 | −4.32 | 0 | 0.01 | −1.26 | 0.22 | 0.49 |
| 7763 | f7H6uyh3Lvfq7Pk6nk | ZFAND5 | 7.98 | 2.42 | 0.03 | 0.08 | −4.62 | 0.01 | 2.03 |
| | 9..STABB9Ioh6o.QJ0 | | 7.98 | −7.37 | 0 | 0 | 3.38 | 0.97 | 0.49 |
| | owq4IDNdU7RBb6ipqo | | 8.23 | −7.04 | 0 | 0 | 2.94 | 0.95 | 0.49 |
| 8939 | oudu78lxv.dOXU8Uvk | FUBP3 | 7.75 | 4.01 | 0 | 0.01 | −1.8 | 0.14 | 2.03 |
| | frx_4TjExSfrLoe6qI | | 8.58 | −5.15 | 0 | 0 | 0.13 | 0.53 | 0.49 |
| 83889 | xEK7hUIrjSEQoLjlCo | WDR87 | 8.16 | −7.28 | 0 | 0 | 3.26 | 0.96 | 0.49 |
| 57149 | EVeQqyLSuItX64tP7E | LYRM1 | 7.37 | 12.1 | 0 | 0 | 8.41 | 1 | 2.03 |
| | ulSctRNoko6BUj70K0 | | 8.04 | −6.26 | 0 | 0 | 1.84 | 0.86 | 0.49 |
| 55364 | EjfXlCv_g8Su4lFOXo | IMPACT | 8.11 | 2.49 | 0.03 | 0.07 | −4.5 | 0.01 | 2.03 |
| 900 | 6nngsu.KNdTpLy4Owg | CCNG1 | 8.08 | 4.76 | 0 | 0 | −0.52 | 0.37 | 2.03 |
| | xupWoEp4p4rpdfgbqM | | 7.57 | 4.81 | 0 | 0 | −0.44 | 0.39 | 2.03 |
| 90410 | 6rpuwoOSVXghJHELeo | IFT20 | 8.42 | 9.05 | 0 | 0 | 5.42 | 1 | 2.03 |
| 2001 | 05Vyh979FIkoNC64pE | ELF5 | 8.08 | −5.42 | 0 | 0 | 0.56 | 0.64 | 0.49 |
| 134637 | 3K09J9eLFE9VJx8hGo | ADAT2 | 8.4 | −4.63 | 0 | 0 | −0.73 | 0.33 | 0.49 |
| 2618 | xU75QpS3gNep0LjXXk | GART | 8.85 | 2.76 | 0.02 | 0.05 | −4.03 | 0.02 | 2.03 |
| 9536 | N5VZ4Z9X5b6f6O3_eQ | PTGES | 7.94 | −8.04 | 0 | 0 | 4.24 | 0.99 | 0.49 |
| 54517 | r6EKf33NLJvxX_0Uuk | PUS7 | 7.77 | 7.16 | 0 | 0 | 3.1 | 0.96 | 2.02 |
| | Qfp0P1XXg4Ibc91A9I | | 7.47 | 7.16 | 0 | 0 | 3.1 | 0.96 | 2.02 |
| | uFd5haKHOVcdWgldoQ | | 7.66 | 9.13 | 0 | 0 | 5.51 | 1 | 2.02 |
| 1350 | f64rllP03tdPd5l.ZI | COX7C | 7.54 | 7.23 | 0 | 0 | 3.2 | 0.96 | 2.02 |
| 60490 | cKn7ebP6J4p4p6fXlc | PPCDC | 7.66 | 5.83 | 0 | 0 | 1.2 | 0.77 | 2.02 |
| 22856 | EEbT6Knmz_wMl450.o | CHSY1 | 8.33 | 2.52 | 0.03 | 0.07 | −4.44 | 0.01 | 2.02 |
| 10096 | lgiwIQi76FLPkvey7Q | ACTR3 | 8.05 | 5.27 | 0 | 0 | 0.32 | 0.58 | 2.02 |
| 643236 | c_BcCiVd8B_WgJpBao | KSP37 | 8.68 | −4.64 | 0 | 0 | −0.72 | 0.33 | 0.49 |
| | fhr7fOJR1ejoKj_KOo | | 8.86 | −4.42 | 0 | 0.01 | −1.09 | 0.25 | 0.5 |
| 6502 | fLhCJ6ryjWvod4TaOU | SKP2 | 7.71 | 4.88 | 0 | 0 | −0.31 | 0.42 | 2.02 |
| 5203 | Huik7on8xASpIFEggY | PFDN4 | 7.71 | 5.47 | 0 | 0 | 0.65 | 0.66 | 2.02 |
| 8562 | 3t7554CO3u5ezQTuH0 | DENR | 8.01 | 2.6 | 0.02 | 0.06 | −4.32 | 0.01 | 2.02 |
| 92312 | 6nf.tcgFWcv6jrJt6o | MEX3A | 8.65 | −5.43 | 0 | 0 | 0.58 | 0.64 | 0.5 |
| 10724 | BRK4e4BI5V635.TR3I | MGEA5 | 7.87 | 5.04 | 0 | 0 | −0.05 | 0.49 | 2.02 |
| 58533 | WVKkr_fhd5dL3YdwKE | SNX6 | 7.63 | 13.09 | 0 | 0 | 9.21 | 1 | 2.02 |
| 5884 | u4QWXbpXaWl3gipLio | RAD17 | 8.62 | −4.02 | 0 | 0.01 | −1.79 | 0.14 | 0.5 |
| 81537 | HKhb9Uw.qXOc174.84 | SGPP1 | 7.84 | 3.17 | 0.01 | 0.03 | −3.29 | 0.04 | 2.02 |
| 283489 | B1x16l8Ttl76p8IUr4 | ZNF828 | 7.8 | 6.68 | 0 | 0 | 2.44 | 0.92 | 2.02 |
| | uskAm6rAV61UCCruPo | | 8.05 | −5.92 | 0 | 0 | 1.33 | 0.79 | 0.5 |
| | olR2eToSCeXP0Lyu9M | | 8.17 | −5.72 | 0 | 0 | 1.03 | 0.74 | 0.5 |
| 2355 | iJJIOJSiySiOpAgB6o | FOSL2 | 8.74 | −5.15 | 0 | 0 | 0.14 | 0.53 | 0.5 |
| 79022 | cO6p_PRX01Pp6BOj6o | TMEM106C | 8.28 | 3.12 | 0.01 | 0.03 | −3.38 | 0.03 | 2.01 |
| 221710 | NlPiS10ivOh3QmHDoM | LOC221710 | 7.77 | 6.48 | 0 | 0 | 2.16 | 0.9 | 2.01 |
| | Tv1RK96KJUo6PPiJOo | | 8.33 | −5.64 | 0 | 0 | 0.9 | 0.71 | 0.5 |
| 2530 | QiF7uBz4gjaBJ18d4o | FUT8 | 7.52 | 5.07 | 0 | 0 | 0 | 0.5 | 2.01 |
| 7546 | fluUC3V9799JE_FTJE | ZIC2 | 7.47 | 10.48 | 0 | 0 | 6.92 | 1 | 2.01 |
| 5985 | BAZH7.TkomqQsK_JeE | RFC5 | 7.8 | 9.21 | 0 | 0 | 5.59 | 1 | 2.01 |
| 196527 | 6V6oD46u6S6F7giog0 | TMEM16F | 7.49 | 7.42 | 0 | 0 | 3.45 | 0.97 | 2.01 |
| | lARX13TEiIRAR8OUeo | | 8.72 | −3.9 | 0 | 0.01 | −1.99 | 0.12 | 0.5 |
| | ovdKUjvouKi5_zSgCo | | 8.64 | −3.99 | 0 | 0.01 | −1.83 | 0.14 | 0.5 |
| 6202 | oA0kt16KJL1JKkJ9_Y | RPS8 | 9.06 | 6.93 | 0 | 0 | 2.8 | 0.94 | 2.01 |
| 51444 | f.Vd._0uT6gyoyH8SM | RNF138 | 7.66 | 7.15 | 0 | 0 | 3.09 | 0.96 | 2.01 |
| | cSCXKEiRMqCME9QRKo | | 8.53 | −4.46 | 0 | 0.01 | −1.02 | 0.26 | 0.5 |
| 25988 | cq3llSjosQ67jfp3_Q | MIZF | 7.9 | 2.99 | 0.01 | 0.04 | −3.62 | 0.03 | 2.01 |
| | 0RHfQRhfYh4oJS0U4o | | 7.96 | −6.56 | 0 | 0 | 2.27 | 0.91 | 0.5 |
| 124801 | 6mnkdUI6addamLH0m8 | LSM12 | 7.47 | 4.57 | 0 | 0 | −0.83 | 0.3 | 2.01 |
| | QKV00jqdXjfoddJf6E | | 7.45 | 11.15 | 0 | 0 | 7.56 | 1 | 2 |
| 6170 | ldd9f3WU267vfh1nnY | RPL39 | 7.49 | 6.48 | 0 | 0 | 2.16 | 0.9 | 2 |
| | ZUwgqdQJBKCU66Irio | | 8.58 | −5.39 | 0 | 0 | 0.51 | 0.62 | 0.5 |
| 57708 | c_Rx.9Lr361X8UTfq4 | MIER1 | 7.6 | 6.36 | 0 | 0 | 1.99 | 0.88 | 2 |
| 94234 | r5KQanEn88uvdwe63U | FOXQ1 | 7.88 | 3.07 | 0.01 | 0.03 | −3.47 | 0.03 | 2 |
| 5504 | Zk5Qkk50uq.yntE5J4 | PPP1R2 | 7.78 | 8.23 | 0 | 0 | 4.46 | 0.99 | 2 |
| 1810 | QHzs9FCVADI_6dUo9Q | DR1 | 7.46 | 10.25 | 0 | 0 | 6.69 | 1 | 2 |
| | BCDF50nUiEbAqEiPqo | | 8.53 | −4.32 | 0 | 0.01 | −1.25 | 0.22 | 0.5 |
| 54948 | c56kxoCx4lTOH4VEKo | MRPL16 | 8.59 | −5.05 | 0 | 0 | −0.03 | 0.49 | 0.5 |

While the preferred embodiments of the invention have been illustrated and described in detail, it will be appreciated by those skilled in the art that that various changes can be made therein without departing from the spirit and scope of the invention. Accordingly, the particular arrangements disclosed are meant to be illustrative only and not limiting as to the scope of the invention, which is to be given the full breadth of the appended claims and any equivalent thereof.

All references, patents, or applications cited herein are incorporated by reference in their entirety, as if written herein.

### REFERENCES

1. Dietel M, Sers C: Personalized medicine and development of targeted therapies: The upcoming challenge for diagnostic molecular pathology. A review. Virchows Arch 2006, 448(6):744-755.
2. Mischel P S, Cloughesy T F, Nelson S F: DNA-microarray analysis of brain cancer: molecular classification for therapy. Nat Rev Neurosci 2004, 5(10):782-792.
3. Muss H B: Targeted therapy for metastatic breast cancer. N Engl J Med 2006, 355(26):2783-2785.
4. Gygi S P, Rochon Y, Franza B R, Aebersold R: Correlation between protein and mRNA abundance in yeast. Mol Cell Biol 1999, 19(3):1720-1730.
5. Ideker T, Thorsson V, Ranish J A, Christmas R, Buhler J, Eng J K, Bumgarner R, Goodlett D R, Aebersold R, Hood L: Integrated genomic and proteomic analyses of a systematically perturbed metabolic network. Science 2001, 292(5518):929-934.
6. Khabar K S, Bakheet T, Williams B R: AU-rich transient response transcripts in the human genome: expressed sequence tag clustering and gene discovery approach. Genomics 2005, 85(2):165-175.
7. Khabar K S: The AU-rich transcriptome: more than interferons and cytokines, and its role in disease. J Interferon Cytokine Res 2005, 25(1):1-10.
8. Intine R V, Tenenbaum S A, Sakulich A L, Keene J D, Maraia R J: Differential phosphorylation and subcellular localization of La RNPs associated with precursor tRNAs and translation-related mRNAs. Mol Cell 2003, 12(5):1301-1307.
9. Tenenbaum S A, Carson C C, Lager P J, Keene J D: Identifying mRNA subsets in messenger ribonucleoprotein complexes by using cDNA arrays. Proc Natl Acad Sci USA 2000, 97(26):14085-14090.
10. Tenenbaum S A, Lager P J, Carson C C, Keene J D: Ribonomics: identifying mRNA subsets in mRNP complexes using antibodies to RNA-binding proteins and genomic arrays. Methods 2002, 26(2):191-198.
11. Keene J D: Organizing mRNA export. Nat Genet 2003, 33(2):111-112.
12. Keene J D, Tenenbaum S A: Eukaryotic mRNPs may represent posttranscriptional operons. Mol Cell 2002, 9(6):1161-1167.
13. Gerber A P, Herschlag D, Brown P O: Extensive association of functionally and cytotopically related mRNAs with Puf family RNA-binding proteins in yeast. PLoS Biol 2004, 2(3):E79.
14. Grigull J, Mnaimneh S, Pootoolal J, Robinson M D, Hughes T R: Genome-wide analysis of mRNA stability using transcription inhibitors and microarrays reveals posttranscriptional control of ribosome biogenesis factors. Mol Cell Biol 2004, 24(12):5534-5547.
15. Hieronymus H, Silver P A: Genome-wide analysis of RNA-protein interactions illustrates specificity of the mRNA export machinery. Nat Genet 2003, 33(2):155-161.
16. Hieronymus H, Yu M C, Silver P A: Genome-wide mRNA surveillance is coupled to mRNA export. Genes Dev 2004, 18(21):2652-2662.
17. Rajasekhar V K, Holland E C: Postgenomic global analysis of translational control induced by oncogenic signaling. Oncogene 2004, 23(18):3248-3264.
18. Atasoy U, Watson J, Patel D, Keene J D: ELAV protein HuA (HuR) can redistribute between nucleus and cytoplasm and is upregulated during serum stimulation and T cell activation. J Cell Sci 1998, 111 (Pt 21):3145-3156.
19. Fan X C, Steitz J A: Overexpression of HuR, a nuclear-cytoplasmic shuttling protein, increases the in vivo stability of ARE-containing mRNAs. Embo J 1998, 17(12):3448-3460.
20. Ma W J, Cheng S, Campbell C, Wright A, Furneaux H: Cloning and characterization of HuR, a ubiquitously expressed Elav-like protein. J Biol Chem 1996, 271(14):8144-8151.
21. Meisner N C, Hackermuller J, Uhl V, Aszodi A, Jaritz M, Auer M: mRNA openers and closers: modulating AU-rich element-controlled mRNA stability by a molecular switch in mRNA secondary structure. Chembiochem 2004, 5(10):1432-1447.
22. Brennan C M, Steitz J A: HuR and mRNA stability. Cell Mol Life Sci 2001, 58(2):266-277.
23. Hanahan D, Weinberg R A: The hallmarks of cancer. Cell 2000, 100(1):57-70.
24. Lopez de Silanes I, Lal A, Gorospe M: HuR: post-transcriptional paths to malignancy. RNA Biol 2005, 2(1):11-13.
25. Abdelmohsen K, Lal A, Kim H H, Gorospe M: Posttranscriptional orchestration of an anti-apoptotic program by HuR. Cell Cycle 2007, 6(11):1288-1292.
26. Abdelmohsen K, Pullmann R, Jr., Lal A, Kim H H, Galban S, Yang X, Blethrow J D, Walker M, Shubert J, Gillespie D A, Furneaux H, Gorospe M: Phosphorylation of HuR by Chk2 regulates SIRT1 expression. Mol Cell 2007, 25(4):543-557.
27. Lal A, Kawai T, Yang X, Mazan-Mamczarz K, Gorospe M: Antiapoptotic function of RNA-binding protein HuR effected through prothymosin alpha. Embo J 2005, 24(10):1852-1862.
28. Lal A, Mazan-Mamczarz K, Kawai T, Yang X, Martindale J L, Gorospe M: Concurrent versus individual binding of HuR and AUF1 to common labile target mRNAs. EMBO J 2004, 23(15):3092-3102.
29. Levy A P: Hypoxic regulation of VEGF mRNA stability by RNA-binding proteins. Trends Cardiovasc Med 1998, 8(6):246-250.
30. Lopez de Silanes I, Zhan M, Lal A, Yang X, Gorospe M: Identification of a target RNA motif for RNA-binding protein HuR. Proc Natl Acad Sci USA 2004, 101(9):2987-2992.
31. Nabors L B, Gillespie G Y, Harkins L, King P H: HuR, a RNA stability factor, is expressed in malignant brain tumors and binds to adenine- and uridine-rich elements within the 3′ untranslated regions of cytokine and angiogenic factor mRNAs. Cancer Res 2001, 61(5):2154-2161.
32. Sheflin L G, Zou A P, Spaulding S W: Androgens regulate the binding of endogenous HuR to the AU-rich 3′UTRs of HIF-1alpha and EGF mRNA. Biochem Biophys Res Commun 2004, 322(2):644-651.
33. Tran H, Maurer F, Nagamine Y: Stabilization of urokinase and urokinase receptor mRNAs by HuR is linked to its cytoplasmic accumulation induced by activated mitogen-activated protein kinase-activated protein kinase 2. Mol Cell Biol 2003, 23(20):7177-7188.
34. Wang W, Caldwell M C, Lin S, Furneaux H, Gorospe M: HuR regulates cyclin A and cyclin B1 mRNA stability during cell proliferation. EMBO J 2000, 19(10):2340-2350.
35. Wang W, Yang X, Cristofalo V J, Holbrook N J, Gorospe M: Loss of HuR is linked to reduced expression of proliferative genes during replicative senescence. Mol Cell Biol 2001, 21(17):5889-5898.
36. Denkert C, Weichert W, Winzer K J, Muller B M, Noske A, Niesporek S, Kristiansen G, Guski H, Dietel M, Hauptmann S: Expression of the ELAV-like protein HuR is associated with higher tumor grade and increased cyclooxygenase-2 expression in human breast carcinoma. Clin Cancer Res 2004, 10(16):5580-5586.
37. Heinonen M, Bono P, Narko K, Chang S H, Lundin J, Joensuu H, Furneaux H, Hla T, Haglund C, Ristimaki A: Cytoplasmic HuR expression is a prognostic factor in invasive ductal breast carcinoma. Cancer Res 2005, 65(6):2157-2161.
38. Heinonen M, Fagerholm R, Aaltonen K, Kilpivaara O, Aittomaki K, Blomqvist C, Heikkila P, Haglund C, Nevanlinna H, Ristimaki A: Prognostic role of HuR in hereditary breast cancer. Clin Cancer Res 2007, 13(23):6959-6963.
39. Gantt K R, Cherry J, Richardson M, Karschner V, Atasoy U, Pekala P H: The regulation of glucose transporter (GLUT1) expression by the RNA binding protein HuR. J Cell Biochem 2006, 99(2):565-574.
40. Guo X, Hartley R S: HuR contributes to cyclin E1 deregulation in MCF-7 breast cancer cells. Cancer Res 2006, 66(16):7948-7956.
41. Kang S S, Chun Y K, Hur M H, Lee H K, Kim Y J, Hong S R, Lee J H, Lee S G, Park Y K: Clinical significance of glucose transporter 1 (GLUT1) expression in human breast carcinoma. Jpn J Cancer Res 2002, 93(10):1123-1128.
42. Pryzbylkowski P, Obajimi O, Keen J C: Trichostatin A and 5 Aza-2′ deoxycytidine decrease estrogen receptor mRNA stability in ER positive MCF7 cells through modulation of HuR. Breast Cancer Res Treat 2008, 111(1):15-25.
43. Saunus J M, French J D, Edwards S L, Beveridge D J, Hatchell E C, Wagner S A, Stein S R, Davidson A, Simpson K J, Francis G D, Leedman P J, Brown M A: Posttranscriptional regulation of the breast cancer susceptibility gene BRCA1 by the RNA binding protein HuR. Cancer Res 2008, 68(22):9469-9478.
44. Suswam E A, Nabors L B, Huang Y, Yang X, King P H: IL-1beta induces stabilization of IL-8 mRNA in malignant breast cancer cells via the 3′ untranslated region: Involvement of divergent RNA-binding factors HuR, KSRP and TIAR. Int J Cancer 2005, 113(6):911-919.
45. Mazan-Mamczarz K, Hagner P R, Corl S, Srikantan S, Wood W H, Becker K G, Gorospe M, Keene J D, Levenson A S, Gartenhaus R B: Post-transcriptional gene regulation by HuR promotes a more tumorigenic phenotype. Oncogene 2008, 27: 6151-6163.
46. Kim H H, Kuwano Y, Srikantan S, Lee E K, Martindale J L, Gorospe M: HuR recruits let-7/RISC to repress c-Myc expression. Genes & Dev 2009, 23: 1743-1748.
47. Atasoy U, Curry S L, Lopez de Silanes I, Shyu A B, Casolaro V, Gorospe M, Stellato C: Regulation of eotaxin gene expression by TNF-alpha and IL-4 through mRNA stabilization: involvement of the RNA-binding protein HuR. J Immunol 2003, 171(8):4369-4378.
48. Casolaro V, Fang X, Tancowny B, Fan J, Wu F, Srikantan S, Asaki S Y, De Fanis U, Huang S K, Gorospe M, Atasoy U X, Stellato C: Posttranscriptional regulation of IL-13 in T cells: role of the RNA-binding protein HuR. The Journal of allergy and clinical immunology 2008, 121(4):853-859 e854.
49. Smyth G: Limma: linear models for microarray data in: Bioinformatics and computational Biology Solutions. In. Edited by Gentleman R C V, Dudoit S, Irizarry R, Huber W. New York: Springer; 2005.
50. Du P, Kibbe W A, Lin S M: lumi: a pipeline for processing Illumina microarray. Bioinformatics 2008, 24(13):1547-1548.
51. Gentleman R C, Carey V J, Bates D M, Bolstad B, Dettling M, Dudoit S, Ellis B, Gautier L, Ge Y, Gentry J, Hornik K, Hothorn T, Huber W, lacus S, Irizarry R, Leisch F, Li C, Maechler M, Rossini A J, Sawitzki G, Smith C, Smyth G, Tierney L, Yang J Y, Zhang J: Bioconductor: open software development for computational biology and bioinformatics. Genome Biol 2004, 5(10):R80.
52. Team R D C: R: A language and environment for statistical computing. In: ISBN 3-900051-07-0. vol. http://www.r-project.org: R Foundation for Statistical Computing Vienna, Austria; 2006.
53. Smyth G K: Linear models and empirical bayes methods for assessing differential expression in microarray experiments. Stat Appl Genet Mol Biol 2004, 3:Article 3.
54. Benjamini Y, Hochberg, Y.: Controlling the false discovery rate: a practical and powerful approach to multiple testing. Journal of the Royal Statistical Society 1995, Series B 57:289-300(57:289-300.).
55. Consortium T G O: Gene Ontology: tool for the unification of biology. Nat Genetics 2000, 25:25-29.
56. Falcon S, Gentleman R: Using GOstats to test gene lists for GO term association. Bioinformatics 2007, 23(2):257-258.
57. Alexa A, Rahnenfuhrer, J, Lengauer, T: Improved scoring of functional groups from gene expression data by decorrelationg GO graph structure. Bioinformatics 2006, 22:1600-1607.
58. Lafleur M A, Xu D, Hemler M E: Tetraspanin proteins regulate membrane type-1 matrix metalloproteinase-dependent pericellular proteolysis. Mol Biol Cell 2009, 20(7):2030-2040.
59. Nakamoto T, Murayama Y, Oritani K, Boucheix C, Rubinstein E, Nishida M, Katsube F, Watabe K, Kiso S, Tsutsui S, Tamura S, Shinomura Y, Hayashi N: A novel therapeutic strategy with anti-CD9 antibody in gastric cancers. J Gastroenterol 2009, 44(9):889-896.
60. Nishida H, Yamazaki H, Yamada T, Iwata S, Dang N H, Inukai T, Sugita K, Ikeda Y, Morimoto C: CD9 correlates with cancer stem cell potentials in human B-acute lymphoblastic leukemia cells. Biochem Biophys Res Commun 2009, 382(1):57-62.
61. Coticchia C M, Revankar C M, Deb T B, Dickson R B, Johnson M D: Calmodulin modulates Akt activity in human breast cancer cell lines. Breast Cancer Res Treat 2009, 115(3):545-560.
62. Schmitt J M, Abell E, Wagner A, Davare M A: ERK activation and cell growth require CaM kinases in MCF-7 breast cancer cells. Mol Cell Biochem 2009, 335(1-2):155-171.
63. Berchtold M W, Egli R, Rhyner J A, Hameister H, Strehler E E: Localization of the human bona fide calmodulin genes CALM1, CALM2, and CALM3 to chromosomes 14q24-q31, 2p21.1-p21.3, and 19q13.2-q13.3. Genomics 1993, 16(2):461-465.
64. Fischer R, Koller M, Flura M, Mathews S, Strehler-Page M A, Krebs J, Penniston J T, Carafoli E, Strehler E E: Multiple divergent mRNAs code for a single human calmodulin. J Biol Chem 1988, 263(32):17055-17062.
65. Bhattacharyya S N, Habermacher R, Martine U, Closs E I, Filipowicz W: Relief of microRNA-Mediated Translational Repression in Human Cells Subjected to Stress. Cell 2006, 125(6):1111-1124.
66. Vasudevan S, Steitz J A: AU-rich-element-mediated upregulation of translation by FXR1 and Argonaute 2. Cell 2007, 128(6):1105-1118.
67. Leung A K, Sharp P A: microRNAs: a safeguard against turmoil? Cell 2007, 130(4):581-585.
68. Figueroa A, Cuadrado A, Fan J, Atasoy U, Muscat G E, Munoz-Canoves P, Gorospe M, Munoz A: Role of HuR in skeletal myogenesis through coordinate regulation of muscle differentiation genes. Mol Cell Biol 2003, 23(14):4991-5004.
69. van der Giessen K, Di-Marco S, Clair E, Gallouzi I E: RNAi-mediated HuR depletion leads to the inhibition of muscle cell differentiation. J Biol Chem 2003, 278(47):47119-47128.
70. Galban S, Kuwano Y, Pullmann R, Jr., Martindale J L, Kim H H, Lal A, Abdelmohsen K, Yang X, Dang Y, Liu J O, Lewis S M, Holcik M, Gorospe M: RNA-binding proteins HuR and PTB promote the translation of hypoxia-inducible factor 1alpha. Mol Cell Biol 2008, 28(1):93-107.
71. Esquela-Kerscher A, Slack F J: Oncomirs—microRNAs with a role in cancer. Nat Rev Cancer 2006, 6(4):259-269.
72. lorio M V, Ferracin M, Liu C G, Veronese A, Spizzo R, Sabbioni S, Magri E, Pedriali M, Fabbri M, Campiglio M, Ménard S, Palazzo J P, Rosenberg A, Musiani P, Volinia S, Nenci I, Calin G A, Querzoli P, Negrini M, Croce C M: MicroRNA gene expression deregulation in human breast cancer. Cancer Res 2005, 65(16):7065-7070.
73. Ma L, Teruya-Feldstein J, Weinberg R A: Tumour invasion and metastasis initiated by microRNA-10b in breast cancer. Nature 2007, 449(7163):682-688.
74. Ma L, Weinberg R A: Micromanagers of malignancy: role of microRNAs in regulating metastasis. Trends Genet 2008, 24(9):448-456.
75. Tavazoie S F, Alarcon C, Oskarsson T, Padua D, Wang Q, Bos P D, Gerald W L, Massague J: Endogenous human microRNAs that suppress breast cancer metastasis. Nature 2008, 451(7175):147-152.
76. Hostetter C, Licata L A, Witkiewicz A, Costantino C L, Yeo C J, Brody J R, Keen J C: Cytoplasmic accumulation of the RNA binding protein HuR is central to tamoxifen resistance in estrogen receptor positive breast cancer cells. Cancer Biol Ther 2008, 7(9).
77. Maglott D, Ostell J, Pruitt K D, Tatusova T: Entrez Gene: gene-centered information at NCBI. Nucleic Acids Res 2005, 33(Database issue):D54-58.
78. Du P, Feng G, Kibbe W, Lin S: lumiHumanAll.db: Illumina Human Expression BeadChips (include all versions: version 1 to 4) annotation data (chip lumiHumanAll). R package version 1.12.0.
79. Carlson M, Falcon, S, Pages, H, Li, N: GO.db: A set of annotation maps describing the entire Gene Ontology. R package version 2.4.5.
80. Gubin M M, Calaluce R, Davis J W, Magee J D, Strouse C S, Shaw D P, Ma L, Brown A, Hoffman T, Rold T L, Atasoy U: Overexpression of the RNA binding protein HuR impairs tumor growth in triple negative breast cancer associated with deficient angiogenesis. Cell Cycle 2010, 9(16):3337-46.
81. Barringer K J, Orgel L, Wahl G and Gingeras T R: Blunt-end and single-strand ligations by Escherichia coil ligase: influence on an in vitro amplification scheme. Gene 1990, 89(1): 117-122.

What is claimed is:

1. An HuR-associated biomarker selected from the group consisting of CALM2, CD9, SRRM1, CCN1, DAZAP2, ARL6IP1, PTMA, ATP1B1, MMD and TMCO1, wherein the level of expression of the HuR-associated biomarker is over- or under-expressed in a breast cancer sample compared to a standard level of expression of the same biomarker in a non-cancerous sample.

2. A set of HuR-associated biomarkers comprising at least one biomarker selected from the group consisting of CALM2, CD9, SRRM1, CCN1, DAZAP2, ARL6IP1, PTMA, ATP1B1, MMD and TMCO1, wherein the level of expression at least one biomarker is over- or under-expressed in a breast cancer sample compared to a standard level of expression of the same biomarker in a non-cancerous sample.

3. The set of claim 2, wherein said standard level of expression is the median expression level of the biomarker in a non-cancerous sample obtained from one or more samples obtained from a population of healthy subjects.

4. The set of claim 2, wherein said standard level of expression is the median expression level of the biomarker in a non-cancerous sample obtained from one or more samples obtained from a subject having breast cancer.

5. The set of claim 2, wherein the ratio of expression of at least one biomarker expressed in the breast cancer sample compared to the non-cancerous sample is less than ½ or greater than 2.

6. The set of claim 2, wherein said breast cancer is an estrogen receptor positive breast cancer.

7. The set of claim 2, wherein said breast cancer is an estrogen receptor negative breast cancer.

8. The set of claim 2, wherein at least one of said biomarkers is an mRNA.

9. The set of claim 2, wherein at least one of said biomarkers is a polypeptide.

10. The set of claim 2, wherein at least one of said biomarkers is post-transcriptionally regulated.

11. The set of claim 2, further comprising at least one biomarker selected from the group consisting of Prothymosin-α, Bcl-2, Mcl-1, SirT1, TGF-b, MMP-9, MTC-1, μPA, VEGF-α, HIF1-α and cyclins A1 (CCNA1), B1 and D1.

12. The set of claim 2, further comprising at least one biomarker selected from the group consisting of Glut-1, ERα, COX-2, IL-8, Cyclin E1, BRCA-1 and Thrombospondin 1.

13. The set of claim 2, further comprising at least one biomarker selected from the group consisting of CD9, PTMA, UBE2E2, CCNI, CKLF, SRRM1, STK4, FKBP1A, PMP22, CALM2, MMD, CSDA, CHIC2, DAZAP2, ZNF22, ATP1B1, TRAM1, ENY2, ALKBH5, RAP2A, TMCO1, and ARL6IP1.

14. The set of claim 2, further comprising at least one biomarker selected from the group consisting of ACTB, SMNDC1, MAL2, CALM2, CDK2AP1, hCG_{—}1781062, JUND, ARL6IP1, PTMA, ATP6V1G1, ACTB, HMGB1, BUB3, PJA2, LOC203547, NPM1, MATR3, TMC01, CXCR7, ZFP36L1, SFRS2, TMSL3, PLOD2, PPP6C, EIF4A2, RPS6KB1, HSPA1A, TIMEM59, FOXA1, PEX11B, MYB, CD9, ZNF14, ITGB1, PARD6B, LOC441087, SRRM1, SNX16, PUM1, MORF4L1, TFDP1, MMD, GCA, CISD2, C4orf34, DAZAP2, G3BP1, C21orf55, NCOA3, ATP1B1, SFPQ, PRKAR1A, YBX1, HIST1H3E, CCNI, CSTB, C15orf51, YWHAZ, PRIM2, SLC7A1, C15orf15, PCBP2, ROD1, SPINT2, CALMG, and YTHDC1.

15. A kit for measuring the level of expression of a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, wherein the level of expression at least one biomarker is over- or under-expressed in a breast cancer sample compared to a standard level of expression of the same biomarker in a non-cancerous sample.

16. A method for aiding in the diagnosis of breast cancer in a subject comprising:
(a) obtaining a sample from said subject;
(b) measuring the level of expression of a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, in said sample obtained from the subject; and
(c) comparing the level of expression of each biomarker in the set of HuR-associated biomarkers to the standard level of expression of the same biomarker in a non-cancerous sample;
wherein a significant difference in the ratio of expression of at least one biomarker in the set aids in the diagnosis of breast cancer.

17. The method of claim 16, wherein said standard level of expression is the median expression level of the biomarker in a non-cancerous sample obtained from one or more samples obtained from a population of healthy subjects.

18. The method of claim 16, wherein said standard level of expression is the median expression level of the biomarker in a non-cancerous sample obtained from one or more samples obtained from a subject having breast cancer.

19. A method for monitoring the disease status of breast cancer in a subject comprising:
(a) obtaining a sample from said subject;
(b) measuring the level of expression of a set of HuR-associated biomarkers comprising at least one biomarker selected from the group consisting of CALM2, CD9, SRRM1, CCN1, DAZAP2, ARL6IP1, PTMA, ATP1B1, MMD and TMCO1, in said sample obtained from the subject; and
(c) comparing the level of expression of each biomarker in the set of HuR-associated biomarkers to the standard level of expression of the same biomarker in a non-cancerous sample;
wherein a significant difference in the ratio of expression of at least one biomarker in the set aids in monitoring the disease status of breast cancer.

20. The method of claim 19, wherein the ratio of expression of at least one biomarker expressed in the cancer sample compared to the non-cancerous sample is less than ½ or greater than 2.
